(19) United States
(12) Patent Application Publication
(10) Pub. No.: US 2021/0071244 A1
(43) Pub. Date: Mar. 11, 2021

(54) METHOD FOR THE AMPLIFICATION OF A NUCLEIC ACID WITH IMPROVED SPECIFICITY

(71) Applicant: AGCT GMBH, Lübeck (DE)

(72) Inventors: Dmitry CHERKASOV, Marburg (DE); Christian GRUNWALD, Gießen (DE)

(73) Assignee: AGCT GMBH, Lübeck (DE)

(21) Appl. No.: 16/976,128

(22) PCT Filed: Feb. 28, 2019
(86) PCT No.: PCT/EP2019/055060

§ 371 Date: Nov. 23, 2020

(30) Foreign Application Priority Data
Feb. 28, 2018 (EP) 18159155.3

(51) Int. Cl. C12Q 1/6848; C12Q 1/686

(52) U.S. Cl. CPC C12Q 1/6848; C12Q 1/686

(57) ABSTRACT

The invention relates to a method for amplification of a nucleic acid by a nucleic acid polymerase via two primers, each of which comprise a sequence segment which is bound to the target sequence and a sequence segment which does not bind to the target sequence, wherein the second cannot serve as a template for the polymerase, and two controller oligonucleotides, each of which is complementary to the primers and a segment of the sequence segment synthesized by them and serves to release the synthesized strand from the template. The controllers also comprise modified nucleotide building blocks such that they cannot serve as templates for the activity of the first template-dependent nucleic acid polymerase.

The present invention relates to a method for amplifying nucleic acids with improved specificity.

The present application claims the priority of the following application: European patent application EP18159155.3 dated 28 Feb. 18; this and all other patent documents mentioned herein shall be deemed to be incorporated by reference in the present description (incorporation by reference).

The nucleic acid sequences provided herewith are shown using standard letter abbreviations for nucleotide bases as defined in 37 C.F.R. 1.822. Only one strand of each nucleic acid sequence is shown, but the complementary strand is understood as included by any reference to the displayed strand. The Sequence Listing is submitted as an ASCII text file named 95083_371_2_seqlist, created Nov. 23, 2020, about 13.6 KB, which is incorporated by reference herein.

### BACKGROUND

The synthesis of nucleic acid chains plays a central role in biotechnology today. Methods such as PCR have significantly advanced both the research landscape and industrial fields of application, such as diagnostics and the food industry. The combination of PCR with technologies such as sequencing, real-time detection, microarray technology, microfluidic management, etc. has contributed to technological development and has partially overcome disadvantages of the original PCR. Other methods, such as isothermal amplification techniques, have also been developed. Their use was specifically intended for the regions of POCT (point-of-care-testing). Despite enormous progress in said field, PCR plays the central role and thus determines the individual technological barriers of its application.

During the amplification process, PCR does not control the amplified sequence segments between the primers. Primer binding is the focus of optimizations of PCR methods. At the beginning of the PCR and during its course, the synthesis of main products and by-products is continuously initiated, e.g. by non-specific binding or extension of the primers. During any back-synthesis reactions that may take place, the unspecifically extended primer is read off as a template, which generally leads to the formation of a complete primer binding site. Thus, a transfer of faulty sequence information from one synthesis cycle to the next takes place, which in sum leads not only to the initial formation, but above all to the exponential increase of by-products.

Such side reactions can lead to exponential propagation of fragments, which interfere with the main reaction (amplification of a target sequence) or lead to interference in subsequent steps of the analysis. Such by-products typically comprise left and right primer sequences, so that their amplification can take place in parallel with the main reaction. However, instead of a target sequence, such by-products comprise a different nucleic acid target sequence.

In order to optimize the synthesis of the main products and to minimize the generation of by-products, primer sequences are typically optimized and reaction conditions are chosen that support the specificity of primer binding. If by-products are nevertheless initiated, they can generally be amplified in parallel to main products, since during the amplification method all newly formed strands are denatured regardless of their composition. During this step, primer binding sites are regenerated, which is the prerequisite for a new round of synthesis. In methods such as PCR or HDA or SDA, agents are used which do not cause sequence-dependent strand separation (in PCR, a temperature increase is used, in HDA a helicase and ATP, and in SDA a polymerase capable of strand displacement). Consequently, primer binding sites for rebinding and subsequent synthesis reaction are generated in both main products and by-products. After a single initiation, both main products and by-products can be replicated in parallel if the primers used find respective primer binding sites.

Specifically susceptible to such side reactions are amplification methods that have to run under reaction conditions that are sometimes difficult to control (e.g., point-of-care testing) or comprise a strong presence of factors that favor side reactions (e.g., amplification of sequence fragments with minor sequence deviations, such as mutations or SNV, in the presence of high amounts of wild-type sequences. Such analyses can be of importance in forensics, prenatal diagnostics or, for example, for ctDNA detection in liquid biopsy investigations).

Primarily, the specificity of PCR amplification is achieved by optimizing primer binding to the target sequences. For example, additional oligonucleotides can be used, which can bind partially to the primer and thus have a competitive participation in the primer binding to other nucleic acid chains.

Such probes generally bind to a sequence segment of the primer and leave a single-stranded sequence segment free at the primer with which the primer can bind to the target nucleic acid and initiate a synthesis reaction. Primer templates mismatches can be competitively displaced by such oligonucleotides, which improves the specificity of the initiation. Such additional oligonucleotides do not interact with the nucleic acid chain to be amplified in segments between the two primers. However, due to a molar excess of primers, unspecific interactions of primers with templates may occur during amplification, resulting in the formation of a fully functional primer binding site as a result of the synthesis of a complementary strand of the by-product. The presence of such a fully functional primer binding site in the by-product leads to a loss of competitive effect of such additional oligonucleotides on primer binding. Thus, controlling the specificity of primer binding to the template by such oligonucleotides only makes the initiation of a side reaction less likely, but can hardly influence its exponential amplification after a by-product has been formed.

Furthermore, labeled oligonucleotide probes are usually used in detection methods to improve the specificity of the analysis for a specific detection of certain sequences. Said probes essentially do not affect exponential amplification. Generally, probes require a sufficiently high concentration of nucleic acid chains and are therefore effective in the final stage of amplification when sufficient product is available (e.g. Taqman probes or Light-Cycler probes).

Generally, improving the specificity of the synthesis of an amplification method with reduced generation and co-amplification of by-products that differ from the target sequence can contribute to improving diagnostic methods, for example.

Based on this background, the objective of the present invention is to provide a method with improved specificity of the synthesis of target nucleic acid chains in exponential amplification.

A further objective of the invention is to provide means for implementing an exponential amplification method with improved specificity of the synthesis.

A further objective of the invention is to provide means and methods which can verify synthesized sequence segments between primers and influence the efficiency of an amplification reaction or the duration of an amplification reaction of sequences depending on their matching with a sequence specification.

A further objective of the invention is to provide means and methods which allow verification of the synthesized sequences in real time and control the usability of the synthesized sequences in further amplification cycles (real-time controlling/on-line quality control).

A further objective of the invention is to provide means and methods which provide an exponential amplification method for the nucleic acid chain with a feedback function of verification of the contents of nucleic acid chains. The feedback function of verification shall take place in parallel to the exponential amplification. The individual amplification elements generally form a feedback loop.

With the method according to the invention, it should be possible to synthesize or amplify nucleic acid chains with a defined sequence composition.

These objectives are solved by the subjects of the independent claims. Further advantageous configurations of the invention are set forth in the dependent claims and the following description.

### SUMMARY OF THE INVENTION

According to a first aspect of the invention, a method for amplification is provided.

In an embodiment, the method for amplifying a nucleic acid chain, or a nucleic acid chain to be amplified comprising a target sequence and/or its complementary sequence segments, comprises the following steps (FIGS. 1 and 13):
Providing a start nucleic acid chain, wherein the start nucleic acid chain
   comprises a 5′-overhang (M2.1.6) which is essentially uncopyable for a polymerase
   comprises a region which comprises sequence segments complementary to the target sequence (M 2.1.5 to M 2.1.1)
   comprises a 3′-region suitable for binding and extending a first oligonucleotide primer (P1.1) (M2.1.1)
Hybridizing a first oligonucleotide primer (P1.1) to the 3′-terminal region of a start nucleic acid chain (M2.1.1) and/or a second primer extension product (P2.1 E1) comprising complementary sequence segments to a target sequence, wherein the first oligonucleotide primer comprises the following regions:
   a first region (P1.1.1) which can bind sequence-specifically to the 3′ terminal region of the start nucleic acid chain (M2.1.1) and/or to the P2.1-Ext (P2.1 E1) of the nucleic acid chain to be amplified,
   a second region (P1.1.2) (primer overhang) which adjoins the 5′ end of the first region or is connected thereto via a linker, wherein the second region can be bound by a first controller oligonucleotide (C1.1) and remains essentially uncopied by a polymerase used for the amplification under the selected reaction conditions;
Hybridizing a second oligonucleotide primer (P2.1) to the 3′-terminal region (P1.1E1) of the complementary strand of the nucleic acid chain to be amplified, wherein the second oligonucleotide primer comprises the following regions:
   a first region (P2.1.1), which can bind sequence-specifically to the 3′-terminal region of the complementary strand (P1.1E1)
   a second region (P2.1.2) (overhang) which adjoins the 5′ end of the first region or is connected thereto via a linker, wherein the second region can be bound by a second controller oligonucleotide (C2.1) and remains essentially uncopied by a polymerase used for the amplification under the selected reaction conditions;
Extension (extension) of the first oligonucleotide primer (P1.1) by a template-dependent polymerase using the start nucleic acid chain and/or the second primer extension product (P2.1-Ext) as template to obtain a first primer extension product (P1.1-Ext), which in addition to the first oligonucleotide primer comprises a region synthesized by the polymerase; the synthesized first primer extension product comprises the target sequence or its portions
Extension (extension) of the second oligonucleotide primer (P2.1) by a template-dependent polymerase using the first primer extension product (P1.1-Ext) as template to obtain a second primer extension product (P2.1-Ext) which comprises a synthesized region in addition to the second oligonucleotide primer; the synthesized second primer extension product comprises sequence segments complementary to the target sequence or their portions
Binding of a first controller oligonucleotide (C1.1) to the first primer extension product (P1.1-Ext), wherein the first controller oligonucleotide comprises the following regions (FIG. 13):
   a first region (C1.1.1) that can bind to the second region (overhang) of the first primer extension product (P1.1E6),
   a second region (C1.1.2) which is complementary to the first region of the second oligonucleotide primer (P1.1.1), and
   a third region (C1.1.3) which is essentially complementary to at least part of the synthesized region of the first segment of the primer extension product (P1.1E4); wherein the first controller oligonucleotide does not serve as a template for primer extension of the first oligonucleotide primer, and the first controller oligonucleotide binds to complementary sequence segments of the first primer extension product to displace complementary segments of the other strand (segments P2.1E1 and P2.1E2) of the nucleic acid chain to be amplified;
Binding of a second controller oligonucleotide (C2.1) to the second primer extension product (P2.1-Ext) and/or to the start nucleic acid chain, wherein the second controller oligonucleotide comprises the following regions:
   a first region (C2.1.1) that can bind to the second region of the second primer extension product (P2.1E6) and/or to the start nucleic acid chain (M 2.1.6),
   a second region (C2.1.2) which is complementary to the first region of the second oligonucleotide primer (P2.1.1), and
   a third region (C2.1.3) essentially complementary to at least a part of the synthesized region of the second primer extension product (P2.1E4);
   wherein the second controller oligonucleotide does not serve as a template for a primer extension of the second oligonucleotide primer, and the second controller oligonucleotide binds to the primer extension product to displace complementary segments of the other strand (P1.1E1 and P1.1E2) of the nucleic acid chain to be amplified;
      wherein: the double strand comprising P1.1-Ext and P2.1-Ext can be separated under reaction conditions with participation of C1.1 and C2.1
      And the synthesized primer extension products (P1.1-Ext and P2.1-Ext) can serve mutually as templates, and
      the steps can be repeated until the desired amount of the nucleic acid to be amplified has been synthesized.

In a further embodiment of the method, the steps of primer extension are further modified and comprise a displacement of the respective controller oligonucleotide from binding with the respective primer extension product with the participation of the polymerase.

In a further embodiment of the method, the method is further modified and comprises the steps of primer extension: continuation of the reaction under conditions that allow the repetition of steps.

In an embodiment, the method is carried out under conditions that do not allow separation of complementary strands of the nucleic acid to be amplified in the absence of a controller oligonucleotide.

In a further embodiment, the method for amplifying a nucleic acid chain comprises a target sequence, wherein the target sequence comprises the following sequence segments (in 5′-3′ direction): TS4.5, TS4.4, TS4.3, TS4.2, TS4.1 (FIG. 13).

In a further embodiment, the method comprises the provision of a start nucleic acid chain (M2.1), wherein the 5′-overhang (M2.1.6) is not complementary to the target sequence and cannot be copied by the polymerase.

In a further embodiment, the method comprises the provision of a start nucleic acid chain (M2.1), wherein the 5′-overhang (M2.1.6) is not complementary to the target sequence and cannot be copied by the polymerase and can bind to the first region of the second controller oligonucleotide.

In an embodiment, the method comprises the provision of a start nucleic acid chain (M2.1) comprising the following sequence segments (in 5′-3′ direction): M2.1.6, M2.1.5, M2.1.4, M2.1.3, M2.1.2, M2.1.1, wherein M2.1.6 is not complementary to the target sequence and M2.1.5, M2.1.4, M2.1.3, M2.1.2, M2.1.1 comprise essentially complementary sequences to the target sequence.

In a further embodiment, the steps of binding the first controller and the second controller to the primer extension products take place simultaneously or parallel to each other.

In a further embodiment, the first controller oligonucleotide is not identical to the second controller oligonucleotide.

In a further embodiment, each primer extension product comprises a segment that is not complementary to the respective controller.

In a further embodiment, each primer extension product comprises a segment that is not complementary to the respective controller, wherein when both controllers bind to complementary regions of the respective primer extension products, both primer extension products can form a double strand due to their segments that are not complementary to the controller.

In a further embodiment, each primer extension product comprises a segment that is not complementary to the respective controller, wherein when both controllers are bound to complementary regions of the respective primer extension products, both primer extension products can form a double strand due to their segments that are not complementary to the controller and these segments comprise P1.1 E3 and P2.1E3.

In a further embodiment, the first primer extension product comprises a P1.1E3 segment.

In a further embodiment, the second primer extension product comprises a P2.1E3 segment.

In a further embodiment, the P1.1-Ext with its P1.1E3 segment can bind complementarily to P2.1E3 of the P2.1-Ext.

In a further embodiment, the first primer extension product comprises a P1.1E3 segment and the second primer extension product comprises a P2.1E3, which has a length in the range from 0 to 100 nucleotides, particularly from 0 to 50 nucleotides, particularly from 0 to 30 nucleotides, particularly from 0 to 15 nucleotides.

In a further embodiment, the method is carried out under reaction conditions comprising temperatures between 25° C. and about 80° C., particularly between 45° C. and 75° C., particularly between 50° C. and 70° C.

In a further embodiment, the P1.1-Ext with its P1.1E3 segment can bind complementarily to P2.1E3 of the P2.1-Ext and form a double strand, wherein the lengths of the segments P1.1E3 and P2.1 E3 are chosen such that the double strand is not stable under reaction conditions and decays.

In a further embodiment, the method is carried out isothermally.

In a further embodiment at least two different temperatures are used.

In an embodiment, the copying of the polynucleotide tail in the second primer region is caused by a stop region for the polymerase, which is located between the first and second region.

In an embodiment, the third single-strand region of the controller oligonucleotide is essentially complementary to the segment of the polymerase synthesized extension product of the respective primer extension product immediately adjacent to the first region, wherein:

In an embodiment, the third single-stranded region of the controller oligonucleotide is completely complementary to the 5′ segment of the extension product of the respective primer extension product, wherein the length of said complementary sequence segment comprises the following ranges: from at least 3 to 70 nucleotides, particularly from at least 5 to 50 nucleotides, particularly from 5 to 40 nucleotides, further particularly from 5 to 30 nucleotides, particularly from 5 to 20 nucleotides.

In a further embodiment of the method, the method is further modified and comprises: simultaneous amplification of the first and second primer extension product in an exponential reaction using the first and second oligonucleotide primer and the first controller oligonucleotide and the second controller oligonucleotide, wherein the primer extension products formed act as templates for mutual synthesis.

In a further embodiment, the method comprises the provision of a start nucleic acid chain (M2.1) (FIGS. 13 and 20) and the following steps:
Providing a nucleic acid chain comprising a target sequence
Hybridizing a second oligonucleotide primer (P2.1) to the 3′-terminal region (TS4.1) of the target sequence, wherein the second oligonucleotide primer comprises the following regions:
   a first region (P2.1.1), which (essentially) can bind sequence-specifically to the 3′-region of the complementary strand (TS4.1 and/or P1.1E1),
   a second region (P2.1.2) (overhang) which adjoins the 5′ end of the first region or is connected thereto via a linker, wherein the second region can be bound by a second controller oligonucleotide (C2.1) and remains essentially uncopied by a polymerase used for the amplification under the selected reaction conditions;
Extension (extension) of the second oligonucleotide primer (P2.1) by a template-dependent polymerase using the target sequence as template to obtain a start nucleic acid chain M2.1 (FIG. 13), which comprises, in addition to the second oligonucleotide primer, a synthesized region which comprises segments complementary to the target sequence;
separating the strands of the target sequence and the obtained start nucleic acid chain from each other, wherein the start nucleic acid chain is converted into single-stranded form.
Wherein the separation can be done by one of the processes: Thermal denaturation, alkali denaturation, enzymatic strand separation (e.g. helicase), degradation (e.g. RNase degradation).

The term “essentially complementary” in the context of the invention means particularly that the regions of nucleic acid which are complementary to each other do not comprise more than 5, 4, 3, 2, or 1 mismatches.

The above-mentioned steps can be carried out in one batch or in separate batches. If the procedures are to be carried out in one batch, they can be carried out under the same conditions, e.g. isothermal, or under different conditions, e.g. thermocycling. Particularly oligonucleotide primers and controller oligonucleotide are present at the beginning of the reaction. However, sequential addition of individual reagents is possible as well.

Combinations with other amplification methods are also possible, e.g. with PCR, wherein the PCR is first carried out over 1 to 10 cycles, for example, and then the reaction continues under for example isothermal conditions.

In an embodiment of the method according to the invention, it is intended that the third region of the first controller oligonucleotide is essentially complementary to the part of the synthesized region of the first primer extension product which is immediately adjacent to the oligonucleotide primer portion of the first primer extension product. This particularly improves the sequence-specific displacement of the nucleic acid to be amplified.

In an embodiment of the method according to the invention, the part of the synthesized region that is essentially complementary to the third region of the first segment of the controller nucleotide comprises a length in the range from 5 nucleotides to 50 nucleotides.

In a further embodiment of the method according to the invention it is intended that the third region of the second controller oligonucleotide is essentially complementary to the part of the synthesized region of the second primer extension product which is immediately adjacent to the oligonucleotide primer portion of the first primer extension product. This particularly improves the sequence-specific displacement of the complementary strand or the first primer extension product.

In a further embodiment of the method according to the invention, the part of the synthesized region which is essentially complementary to the third region of the second controller nucleotide comprises a length in the range from 3 nucleotides to 70 nucleotides, particularly 5 nucleotides to 50 nucleotides, particularly 5 nucleotides to 50 nucleotides, further particularly 5 nucleotides to 30 nucleotides, further particularly 5 nucleotides to 20 nucleotides.

In an embodiment, the third single-stranded region of the first controller oligonucleotide is completely complementary to the 5′ segment of the first primer extension product, wherein the length of said complementary sequence segment comprises the following ranges: from at least 3 to 70 nucleotides, particularly from at least 5 to 50 nucleotides, particularly from 5 to 40 nucleotides, from 5 to 30 nucleotides, or particularly from 5 to 20 nucleotides.

In an embodiment, the third single-stranded region of the second controller oligonucleotide is completely complementary to the named 5′ segment of the second primer extension product, wherein the length of said complementary sequence segment comprises the following ranges: from at least 3 to 70 nucleotides, particularly from at least 5 to 50 nucleotides, particularly from 5 to 40 nucleotides, further particularly from 5 to 30 nucleotides, or from 5 to 20 nucleotides.

In a further embodiment of the method according to the invention, it is provided that the method further comprises the following step: hybridizing a probe to a region of the nucleic acid to be amplified, the complementary strand, the first primer extension product or the second primer extension product, wherein the region is not bound by or interacts with the first controller oligonucleotide or the second controller oligonucleotide.

In a further embodiment of the method according to the invention, it is intended that the method further comprises the following step: hybridizing a block oligonucleotide to a region of the nucleic acid to be amplified, the complementary strand, the first primer extension product or the second primer extension product, wherein the region is not bound by the first controller oligonucleotide or the second controller oligonucleotide, and the hybridizing of the block oligonucleotide prevents amplification of the nucleic acid to be amplified, the complementary strand, the first primer extension product or the second primer extension product.

In a further embodiment of the method according to the invention, it is intended that the method is essentially isothermal.

The term “essentially isothermal” in the sense of the present description means in particular that the reaction temperature is not changed by more than 10° C. within the method.

In a further embodiment of the method according to the invention, it is intended that the nucleic acid to be amplified comprises a length in the range from 30 nucleotides to 200 nucleotides.

In a further embodiment of the method according to the invention, it is intended that the first segment of the oligonucleotide primer comprises a length in the range from 15 nucleotides to 60 nucleotides.

In a further embodiment of the method according to the invention, it is provided that the second oligonucleotide primer comprises a length in the range from 15 nucleotides to 60 nucleotides.

In a further embodiment of the method according to the invention, it is provided that the first segment of the controller oligonucleotide comprises a length in the range from 20 nucleotides to 100 nucleotides.

In a further embodiment of the method according to the invention, it is provided that the second controller oligonucleotide comprises a length in the range from 20 nucleotides to 100 nucleotides.

In a further embodiment of the method according to the invention, it is provided that the region of the double strand comprising the first primer extension product and the nucleic acid to be amplified, which is not bound by the first controller oligonucleotide or the second controller oligonucleotide, dissociates into the respective single strands under the selected reaction conditions.

In a further embodiment of the method according to the invention, it is provided that the region of the double strand comprising the second primer extension product and the complementary strand which is not bound by the first controller oligonucleotide or the second controller oligonucleotide dissociates into the respective single strands under the selected reaction conditions.

In a further embodiment of the method according to the invention, it is provided that the region of the double strand comprising the first primer extension product and the second primer extension product, which is not bound by the first controller oligonucleotide or the second controller oligonucleotide, dissociates into the respective single strands under the selected reaction conditions.

In a further embodiment of the method according to the invention, it is provided that the selected reaction conditions comprise a reaction temperature in the range from 30° C. to 75° C.

In a further embodiment of the method according to the invention, it is provided that the first oligonucleotide primer comprises one or more modifications in the second region, particularly immediately following the first region of the first oligonucleotide primer, which stop the polymerase used at the second region. Particularly, this means that only the first region of the first oligonucleotide primer is used as a template by a polymerase.

In a further embodiment of the method according to the invention, it is provided that the second oligonucleotide primer comprises one or more modifications in the second region, particularly immediately following the first region of the second oligonucleotide primer, which stop the polymerase used at the second region. Particularly, this means that only the first region of the second oligonucleotide primer is used as a template by a polymerase.

This can particularly be achieved by using nucleotide modifications which can undergo complementary binding of a controller oligonucleotide to the first segment of the respective region, but are not accepted by the polymerase as template. Examples of such nucleotide modifications are nucleotide compounds with modified phosphate-sugar backbone portions, e.g. 2′-O-alkyl RNA modifications (e.g. 2′-OMe), LNA modifications or morpholino modifications. Generally, the presence of such modifications in a strand prevents a DNA-dependent polymerase from reading off such a strand. The number of such modifications can vary, generally several modifications (between 5 and 30) can be sufficient to prevent a polymerase from reading off such a strand.

Thanks to the use of such modifications, the polymerase function is inhibited locally, so that certain segments of the structures used cannot be copied by the polymerase and remain predominantly single-stranded. In said single-stranded form they can bind further reaction components and thus perform their function.

According to a further aspect of the invention, a kit for carrying out the method according to the first aspect or one of the further embodiments or alternatives is provided. The kit according to the invention comprises:
a first oligonucleotide primer comprising the following regions:
   a first region that can bind sequence specifically to the 3′-terminal region of a nucleic acid chain to be amplified,
   a second region which adjoins the 5′ end of the first region or is connected thereto via a linker, wherein the second region can be bound by a first controller oligonucleotide and remains essentially uncopied by a polymerase used for the amplification under the selected reaction conditions;
   wherein the first oligonucleotide primer can be extended by a polymerase to a first primer extension product which comprises besides the first oligonucleotide primer a synthesized region;
a second oligonucleotide primer comprising the following regions:
   a first region that can bind sequence-specifically to the 3′-terminal region of the first primer extension product
   a second region which adjoins the 5′ end of the first region or is connected thereto via a linker, wherein the second region can be bound by a second controller oligonucleotide and remains essentially uncopied by a polymerase used for the amplification under the selected reaction conditions;
   wherein the second oligonucleotide primer can be extended by a polymerase to form a second primer extension product which comprises besides the second oligonucleotide primer a synthesized region;
a first controller oligonucleotide comprising the following regions:
   a first region that can bind to the second region of the first primer extension product
   a second region that is essentially complementary to the first region of the second oligonucleotide primer; and
   a third region that is essentially complementary to at least a part of the synthesized region of the first primer extension product;
      wherein the first controller oligonucleotide does not serve as a template for primer extension of the first oligonucleotide primer; and
a second controller oligonucleotide comprising the following regions:
   a first region that can bind to the second region of the second primer extension product,
   a second region that is essentially complementary to the first region of the second oligonucleotide primer; and
   a third region that is essentially complementary to at least a part of the synthesized region of the second primer extension product;
      wherein the second controller oligonucleotide does not serve as a template for primer extension of the second oligonucleotide primer.

In an embodiment of the kit according to the invention, the kit further comprises a polymerase.

According to a further aspect of the invention, the use of a kit according to the above aspect is provided for carrying out the method according to the first aspect of the invention or one of the further embodiments or alternatives thereof.

Further details and advantages of the invention shall be explained in a non-restrictive manner by the following figures and a detailed description of selected embodiments.

### DESCRIPTION OF THE FIGURES

FIG. 1 shows the sequence components of an embodiment of the amplification method according to the invention.

FIG. 2 shows A: the schematic structure of primer 1.1, primer 2.1, controller 1.1 and controller 2.1 and B: the interaction between primer 1.1 and controller 1.1 or primer 2.1 and controller 2.1.

FIG. 3 shows the topography of the nucleic acid to be amplified (P.1. extension and P2.1 extension):

FIG. 4 shows a schematic representation of primer binding to the primer extension products; A: primer 1.1 binds to PBS P1.1 of the primer 2.1 extension product; B: primer 2.1 binds to PBS P2.1 of the primer 1.1 extension product.

FIG. 5 shows a schematic representation of the controller binding to the primer extension products.

FIG. 6 shows a schematic representation of the interaction between the controllers (C1.1/C1.2) and the primer extension products (P1.1-Ext./P2.1-Ext.).

FIG. 7 shows a schematic representation of the interaction between the controllers C1.1 and C2.1 and the complex comprising P1.1-Ext/P2.1-Ext as well as the use of the complexes P1.1-Ext/C1.1 and P2.1-Ext/C2.1 as templates for polymerase-dependent P1.1 and P2.1 extension.

FIG. 8 shows a schematic representation of the amplification starting from P1.1-Ext.

FIG. 9 shows a schematic representation of the creation of a start nucleic acid chain starting from P1.1 and its use in the amplification.

FIG. 10 shows a schematic representation of a longer double-stranded nucleic acid chain (A); the hybridization of primer P.1.1 to the 3′ end of one strand of the double-stranded nucleic acid chain (B); the extended primer P1.1-Ext (C); and the extension of primer P2.1 hybridized to P1.1-Ext as template (D).

FIG. 11 shows a schematic representation of a longer double-stranded nucleic acid chain (A); the hybridization of primer P2.1 to the 3′ end of one strand of the double-stranded nucleic acid chain (B); the extended primer P2.1-Ext (C); and the extension of primer P1.1 hybridized to P2.1-Ext. as template.

FIG. 12 shows schematically the synthesis of P1.1-Ext. displacing the controller C2.1.

FIG. 13 shows schematically the synthesis of P2.1-Ext. displacing controller C1.1.

FIG. 14 shows schematically the interaction of controller C1.1 and C2.1 with the complex of P1.1-Ext. and P2.1-Ext.

FIG. 15 schematically shows the interaction of controllers C1.1 and C2.1 with the complex of P1.1-Ext. and P2.1-Ext.

FIG. 16 shows schematically the interaction of controllers C1.1 and C2.1 with the complex of P1.1-Ext. and P2.1-Ext.

FIG. 17-19 show schematically certain embodiments of the topography of a nucleic acid chain to be amplified

FIG. 20 shows a schematic representation of the creation of a start nucleic acid chain starting from P2.1 and its use in amplification.

FIG. 21-30 show results of the amplification (examples).

### DETAILED DESCRIPTION OF THE INVENTION

### Definitions and Specific Embodiments

Unless otherwise defined, all technical and scientific terms used herein have the same meaning as they are commonly understood by an average professional. Standard techniques are used for molecular biological or biochemical methods (see e.g. Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.)

In the context of the present invention, the terms used have the following meaning:

If the sequences shown in the description and in the sequence listing differ from each other, the sequence shown in the description applies in case of doubt.

### Oligonucleotide Primer:

The oligonucleotide primer comprises a first region and a second region.

In the present invention two oligonucleotide primers are used, each for initiating the synthesis of a specific primer extension product. Although the first and second oligonucleotide primer differ from each other in the sequence composition of individual regions, the basic structure of both oligonucleotide primers is similar.

Therefore, only the structure of the first oligonucleotide primer is discussed as an example. The construction of the second primer follows essentially the same considerations.

The first primer region is capable of binding to an essentially complementary sequence within the nucleic acid to be amplified or its equivalents and initiating a primer extension reaction. The second region comprises a polynucleotide tail capable of binding a controller oligonucleotide and thereby causing a proximity between the controller oligonucleotide and the other parts of the respective primer extension product sufficient to initiate strand displacement by the controller oligonucleotide. The second region of the oligonucleotide primers further comprises at least one modification (a nucleotide modification or a non-nucleotide modification) that prevents the polymerase from copying the polynucleotide tail by inhibiting the continuation of polymerase-dependent synthesis. This modification is located, for example, at the transition between the first and second regions of the oligonucleotide primer. Consequently, the first region of the oligonucleotide primer is copyable by a polymerase, so that a complementary sequence to this region can be generated by the polymerase during the synthesis of the second primer extension product. The polynucleotide tail of the second region of the oligonucleotide primer is not copied by the polymerase.

### Certain Embodiments of the Oligonucleotide Primer

In an embodiment this is achieved by modification in the second region, which stops the polymerase prior to the polynucleotide tail.

In a further embodiment this is achieved by nucleotide modifications in the second region, wherein the entire polynucleotide tail essentially consists of such nucleotide modifications and is therefore uncopyable for the polymerase.

In an embodiment, each oligonucleotide primer is specific for one nucleic acid to be amplified.

In an embodiment, each first oligonucleotide primer is specific for at least two of the nucleic acids to be amplified, which respectively comprise essentially different sequences.

In an embodiment, the first oligonucleotide primer is labeled with a characteristic label, e.g. a fluorescent dye (e.g. TAMRA, Fluorescein, Cy3, Cy5) or an affinity marker (e.g. biotin, digoxigenin) or an additional sequence fragment, e.g. for binding a specific oligonucleotide probe for detection or immobilization or for barcode labeling.

### Primer Extension Product:

A primer extension product (also called primer extension product) is formed by enzymatic (polymerase-dependent) extension of an oligonucleotide primer as a result of template-dependent synthesis catalyzed by a polymerase.

A primer extension product comprises the sequence of the oligonucleotide primer in its 5′-segment and the sequence of the extension product (also called extension product), which was synthesized by a polymerase in template-dependent form. The extension product synthesized by the polymerase is complementary to the template strand on which it was synthesized.

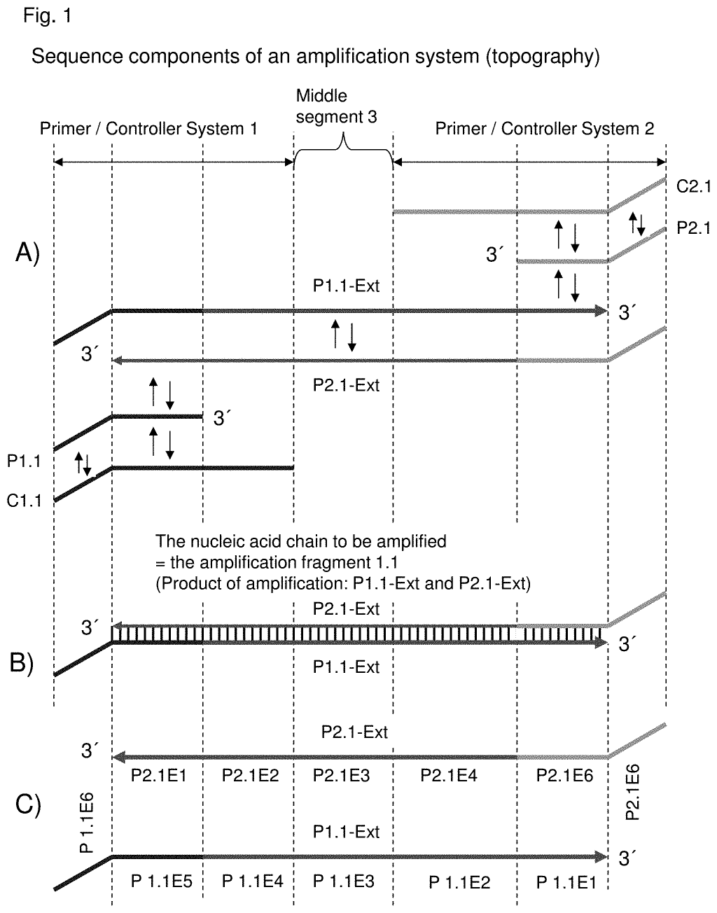

A specific primer extension product (e.g. in FIG. 1 P.1.1-Ext or P2.1-Ext.) (main product of amplification, also called the nucleic acid chain to be amplified, also called amplification fragment) comprises sequences of the nucleic acid chain to be amplified. It is a result of a specific synthesis or correct performance of an intended primer extension reaction in which the specific nucleic acid chain to be amplified serves as a template.

An unspecific primer extension product (by-product) comprises, for example, sequences that are the result of an unspecific, incorrect or unintended primer extension reaction. These include, for example, primer extension products that are the result of an incorrect initiation event (false priming) or other side reactions, including polymerase-dependent sequence changes such as base substitution, deletion, etc. The extent of sequence deviation from unspecific primer extension products generally exceeds the ability of controller oligonucleotides to successfully displace such double-stranded by-products from their templates, resulting in slower or complete absence of amplification of such by-products. The degree of acceptance or tolerance of deviations depends, for example, on the reaction temperature and the nature of the sequence deviation. Examples of non-specific primer extension products are primer dimers or sequence variants that do not correspond to the nucleic acid to be amplified, e.g. sequences that do not comprise a target sequence.

The assessment of sufficient specificity of amplification is often related to the task at hand. In many amplification methods a certain degree of unspecificity of the amplification reaction can be tolerated as long as the desired result can be achieved.

### Certain Embodiments of the Primer Extension Product

In an embodiment, the sequence of the synthesized primer extension products completely matches the expected sequence of a nucleic acid to be amplified.

In another embodiment, deviations in the obtained sequence from the theoretically expected sequence can be tolerated.

In an embodiment, the degree of matching of the obtained sequence as a result of amplification with the sequence of the theoretically expected nucleic acid to be amplified is, for example, between 90% and 100%, particularly the degree of matching is above 95%, particularly the degree of matching is above 98% (measured by the portion of synthesized bases).

The length of the extension product of a specific primer extension product can be between 10 and 300 nucleotides, in some embodiments between 10 and 180 nucleotides, in some embodiments between 20 and 120 nucleotides, or between 30 and 80 nucleotides.

In an embodiment, the portion of nucleic acid chains to be amplified amounts to more than 1% of the total reaction result, particularly more than 10%, particularly more than 30% measured on the total quantity of newly synthesized strands.

### The Nucleic Acid Chain to be Amplified

The nucleic acid chain to be amplified represents a nucleic acid chain which is to be amplified sequence-specifically or at least predominantly sequence-specifically by the polymerase by means of exponential amplification using primers and controller oligonucleotide.

The nucleic acid chain to be amplified comprises the first specific and the second specific primer extension product.

The nucleic acid chain to be amplified comprises a target sequence or is complementary to it.

The length of the nucleic acid chain to be amplified can be between 20 and 300 nucleotides, particularly between 30 and 200 nucleotides, or between 40 and 150 nucleotides, or between 50 and 100 nucleotides.

The nucleic acid chain to be amplified can comprise one or more target sequences or their equivalents. Furthermore, a nucleic acid chain to be amplified can comprise the sequences essentially complementary to a target sequence, which are amplified with similar efficiency as a target sequence in an amplification reaction and comprise the target sequence or its segments. In addition to a target sequence, the nucleic acid to be amplified can include further sequence segments, for example primer sequences, sequences with primer binding sites and/or sequence segments for the binding of detection probes, and/or sequence segments for sequence coding of strands by barcode sequences and/or sequence segments for binding to a solid phase. The primer sequences or their sequence parts as well as primer binding sites or their sequence parts can belong to sequence segments of a target sequence.

In order to start the amplification, a nucleic acid chain (start nucleic acid chain) must be added to the reaction mixture at the beginning of the reaction, which acts as an initial template for the synthesis of the nucleic acid chain to be amplified. Said nucleic acid chain is called the start nucleic acid chain. Said start nucleic acid chain defines the array of individual sequence elements which are important for the formation/synthesis/exponential amplification of a nucleic acid chain to be amplified.

This start nucleic acid chain comprises an uncopyable 5′ region (overhang), which is identical in a certain embodiment with the second primer region of a primer.

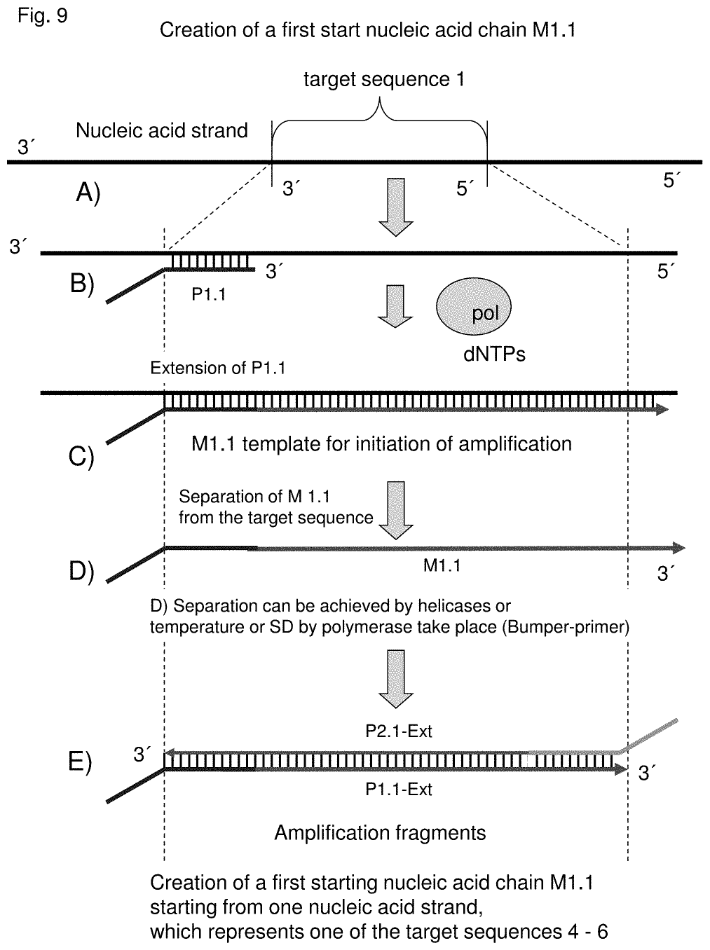

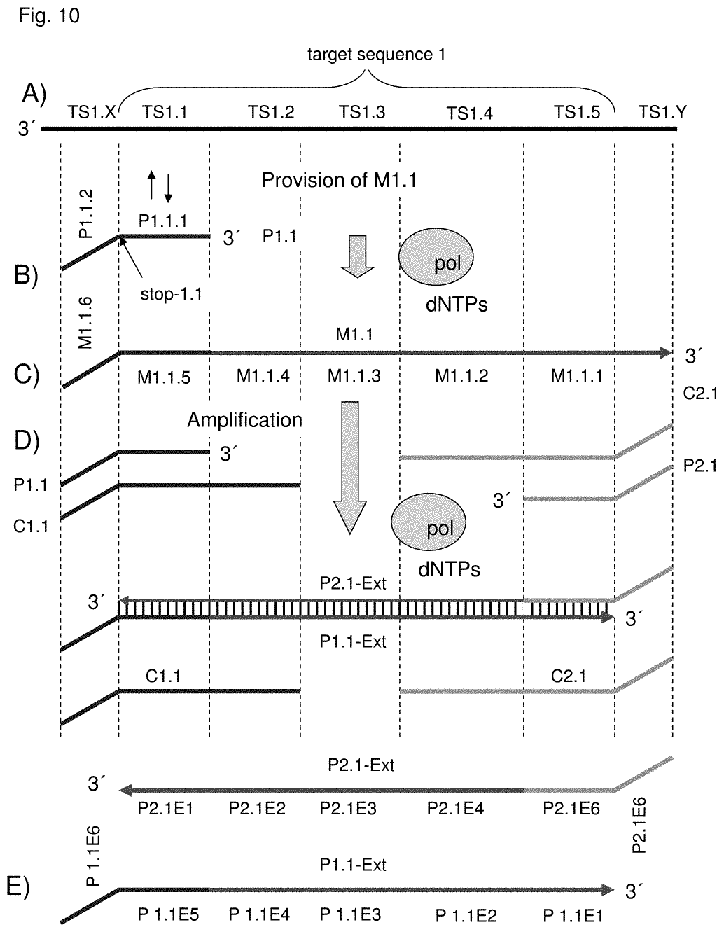

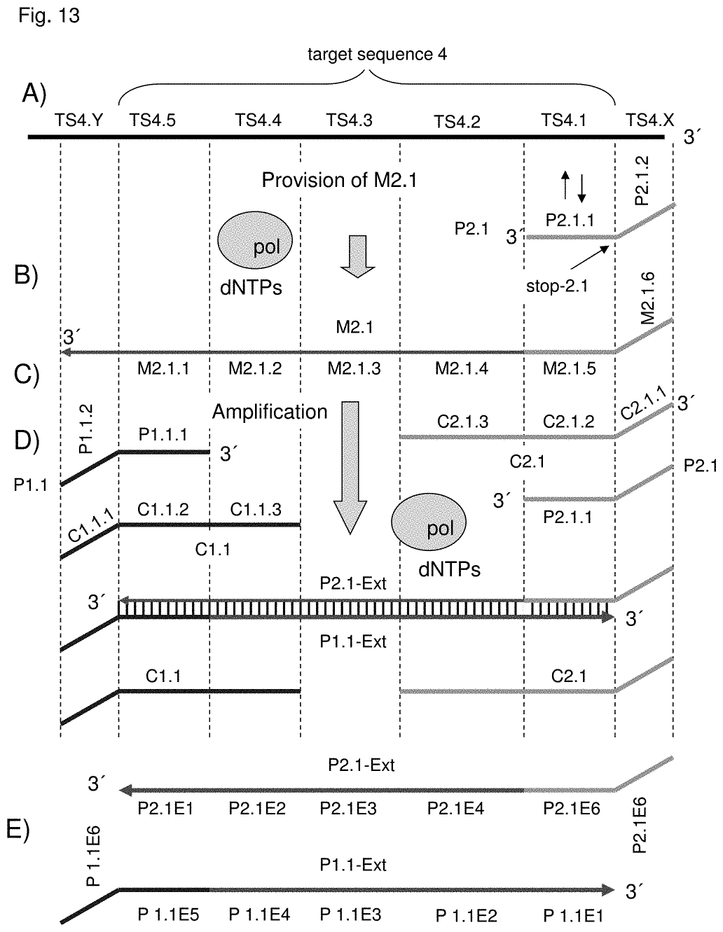

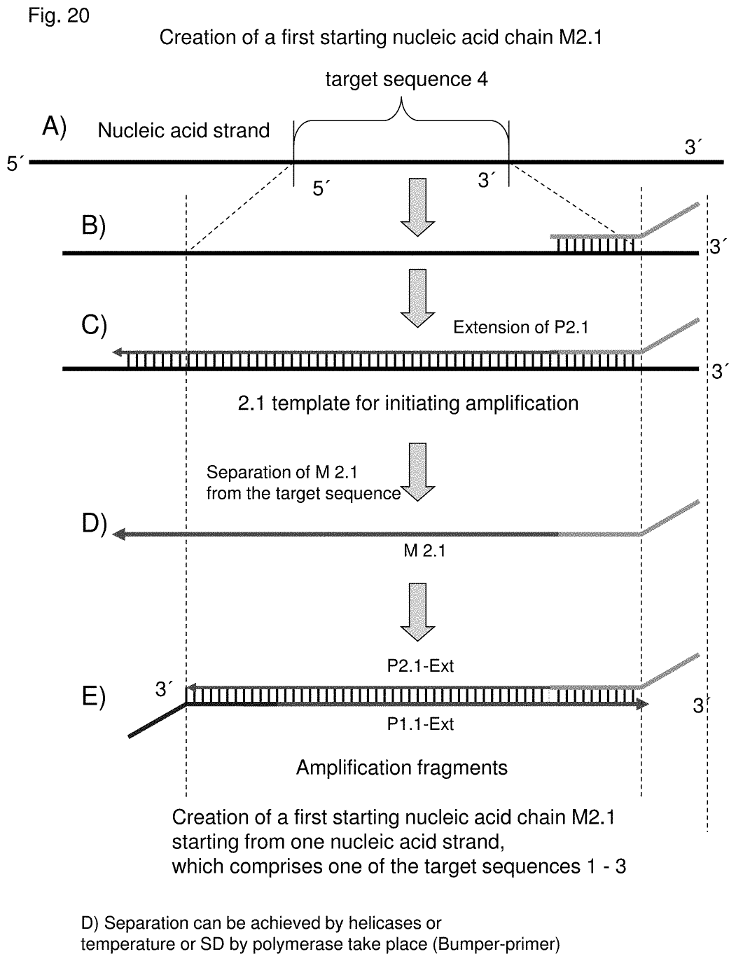

Such a start nucleic acid chain (M1.1 or M2.1) can, for example, be prepared prior to amplification by a primer extension reaction using a nucleic acid strand comprising a target sequence and one of the two amplification primers (the first oligonucleotide primer, FIGS. 9 and 10, or the second oligonucleotide primer, FIGS. 13 and 20) as well as a suitable polymerase and substrates (dNTP). After conversion of such a prepared start nucleic acid chain into a single-stranded form (e.g. by heat denaturation or alkali denaturation or enzymatic degradation of target sequence, e.g. via RNases), such a start nucleic acid chain can be given as initial template to start the reaction.

In initial stages of the amplification reaction and in its further course, the respective primers bind to the respective binding sites in the start nucleic acid chain and initiate the synthesis of specific primer extension products. Such specific primer extension products accumulate exponentially in the course of the amplification and increasingly take over the role of templates for the synthesis of complementary primer extension products in exponential amplification.

The repeated template-dependent synthesis procedures in exponential amplification thus form the nucleic acid chain to be amplified.

Towards the end of an amplification reaction, the main product of the reaction (the nucleic acid to be amplified) may be predominantly single-stranded or predominantly form a complementary double-strand. This can be determined, for example, by the relative concentration of both primers and respective reaction conditions.

Equivalents of the nucleic acid chain to be amplified comprise nucleic acids with essentially identical information content. For example, complementary strands of a nucleic acid to be amplified have identical information content and can be called equivalent.

### Certain Embodiments of the Nucleic Acid to be Amplified

In an embodiment, a nucleic acid to be amplified comprises a target sequence. In an embodiment, the target sequence corresponds to the nucleic acid to be amplified. In a further embodiment, a start nucleic acid chain comprises a target sequence. In an embodiment, the target sequence corresponds to a respective start nucleic acid chain.

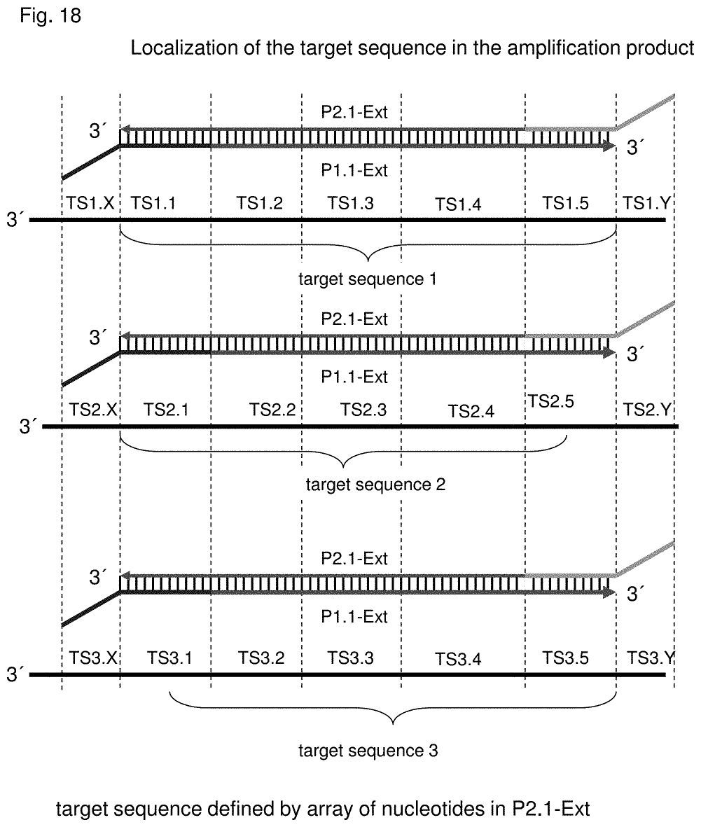

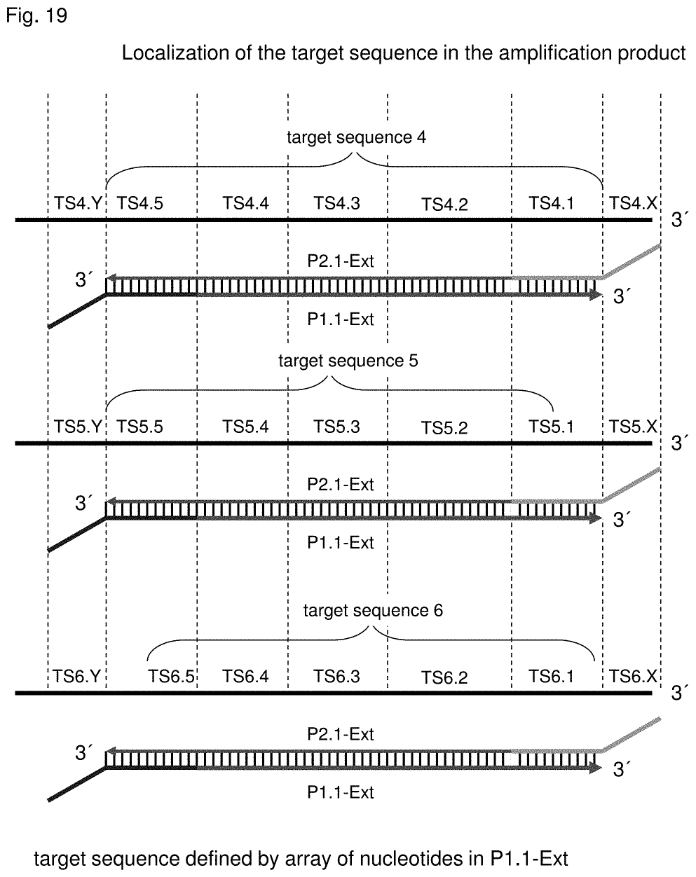

In an embodiment, the target sequence (FIGS. 18 and 19) forms part of the sequence of the nucleic acid chain to be amplified. Such a target sequence can be flanked by 3′ side and/or by 5′ side of further sequences. Said further sequences can comprise, for example, binding sites for primers or their portions, and/or primer sequences or their portions, and/or binding sites for detection probes, and/or adaptor sequences for complementary binding to a solid phase (e.g., in microarrays, or bead-based analyses), and/or barcoding sequences for digital signature of sequences.

In an embodiment, the initial template (start nucleic acid chain), which is added to an amplification reaction at the beginning, or which is added to the reaction mixture, corresponds to the sequence composition of the nucleic acid chain to be amplified.

### Target Sequence

In an embodiment, a target sequence is a segment of a nucleic acid chain to be amplified, which can serve as the characteristic sequence of the nucleic acid to be amplified. Said target sequence can serve as a marker for the presence or absence of another nucleic acid. Thus, said other nucleic acid serves as source of the target sequence and can be, for example, genomic DNA or RNA or parts of genomic DNA or RNA (e.g. mRNA), or equivalents of genomic DNA or RNA of an organism (e.g. cDNA, modified RNA such as rRNA, tRNA, microRNA, etc.).), or comprise defined modifications of the genomic DNA or RNA of an organism, such as mutations (e.g. deletions, insertions, substitutions, additions, sequence propagation, e.g. repeat propagation in the context of microsatellite instability), splice variants, rearrangement variants (e.g. T-cell receptor variants), etc. The individual target sequences may represent a phenotypic trait, e.g. antibiotic resistance or have prognostic information and thus be of interest for diagnostic assays/tests. As source/origin of a target sequence, such a nucleic acid can comprise the target sequence as sequence element of its strand. A target sequence can thus serve as a characteristic marker for a specific sequence content of another nucleic acid.

The target sequence can be single-stranded or double-stranded. It can be essentially identical to the nucleic acid to be amplified or it can represent only a part of the nucleic acid to be amplified.

Equivalents of the target sequence comprise nucleic acids with essentially identical information content. For example, complementary strands of a target sequence have identical information content and can be called equivalent, RNA and DNA variants of a sequence are also examples of equivalent information content.

During material preparation prior to the amplification reaction, such a target sequence can be isolated from its original sequence environment and prepared for the amplification reaction.

### Start Nucleic Acid Chain (FIGS. 9 and 10, 13 and 20)

In order for the amplification to start, a nucleic acid chain must be present in the reaction mixture at the beginning of the reaction, which acts as an initial template for the synthesis of the nucleic acid chain to be amplified. Said nucleic acid chain is called the start nucleic acid chain. Said start nucleic acid chain defines the array of individual sequence elements that are important for the formation/synthesis/exponential amplification of a nucleic acid chain to be amplified.

Such a start nucleic acid chain can be single stranded or double stranded at the beginning of the reaction. If the complementary strands of the start nucleic acid chain are separated, the strands can serve as templates for the synthesis of specific complementary primer extension products, regardless of whether the nucleic acid was originally double-stranded or single-stranded.

### Controller Oligonucleotide

The controller oligonucleotide (C1.1 and C2.1) is a single-stranded nucleic acid chain that includes a sequence essentially complementary to a part of a primer extension product that is specifically generated during the amplification of the nucleic acid to be amplified. Thus, the controller oligonucleotide can bind to the first oligonucleotide primer and at least to the 5′ segment of the specific extension product of the first oligonucleotide primer essentially complementarily.

At least two controller oligonucleotides are used in the present invention, wherein each can bind to its primer extension product sequence-specifically. The first controller oligonucleotide specifically interacts with the first oligonucleotide primer and with the first primer extension product. The second controller oligonucleotide specifically interacts with the second oligonucleotide primer and with the second primer extension product.

Although the two controller oligonucleotides used differ from each other, the basic structure of a controller oligonucleotide is the same for both controller oligonucleotides used. Therefore, the first controller oligonucleotide is explained to simplify the representation. The structure of the second controller oligonucleotide can be adapted to the respective specifications.

The first controller oligonucleotide can bind essentially complementary to the first oligonucleotide primer and at least to the 5′-segment of the specific extension product of the first oligonucleotide primer.

The controller oligonucleotide comprises in its inner sequence segment nucleotide modifications that prevent the polymerase from synthesizing a complementary strand using the controller oligonucleotide as template when the first oligonucleotide primer is complementarily bound to the controller oligonucleotide.

The controller oligonucleotide is further capable of displacing the respective specific second primer extension product completely or partially from binding with the specific first primer extension product via strand displacement under the selected reaction conditions. In this process, the controller oligonucleotide with its complementary regions anneals to the specific first primer extension product. If binding of a controller oligonucleotide to the specific first primer extension product is successful, this leads to the restoration of a single-stranded state of the 3′-positioned segment of the second specific primer extension product, which is suitable for binding of the first oligonucleotide, so that a new primer extension reaction can take place. During the synthesis of the second primer extension product, the controller oligonucleotide can be separated from the binding of a controller oligonucleotide with the first primer extension product by strand displacement, for example by the polymerase and/or by the second oligonucleotide primer.

### Detailed Description of Certain Embodiments

The present invention relates particularly to a method for the exponential amplification of DNA, which includes sequence control or sequence verification during amplification over partial segments of the fragment to be amplified by means of controller oligonucleotides. Thus, an improved specificity of the exponential amplification reaction can be achieved. Only sequences that match the predefined controller sequences are amplified. Sequence verification does not take place over the total length of the DNA fragment to be amplified. Middle segments (strand segments which do not interact with any of the controller oligonucleotides) can vary in sequence composition and sequence length.

This provides a possibility to amplify sequence variations even if they are not known exactly, e.g. SNV and InDels in hotspots (like KRAS codon 12, EGFR deletions/insertions in exons 19 and 21).

The method according to the invention starts with the extension of two primers. Immediately after the synthesis, the amplification fragment is present in double-stranded form. Said double strand is stable under the reaction conditions and cannot dissociate spontaneously or said spontaneous dissociation takes place very slowly.

In double-stranded form of the fragment to be amplified, primer-binding sequence segments are also present in double-stranded form and are therefore not accessible for the binding of single-stranded primers. Therefore, amplification fragments with primer binding sites in double-stranded form cannot act as templates for specific sequence synthesis. Thus, the double-stranded form represents an inert form of the amplification fragment.

For a specific exponential amplification, the synthesized sequence fragments should be used as templates in subsequent synthesis cycles. As a prerequisite, the synthesized amplification fragment must be converted from its double-stranded form at least partially into single-stranded form, wherein at least the primer binding sites must be present in single-stranded form and thus be capable of binding primers that can initiate synthesis.

A double-stranded opening takes place with the participation of two controller oligonucleotides, which comprise complementary sequences to terminal regions of the fragment to be amplified. The controller oligonucleotides are thus able to bind to a sequence segment of the synthesized strand and form a double strand. Particularly primer-binding sites are converted into single-stranded form and can bind new primers.

The prerequisite for opening the double strand of the fragment to be amplified is particularly a sequence match with the respective controller sequences. Thus, after each synthesis step, a sequence verification of segments of the synthesized fragment takes place before the synthesized fragment is transferred from a double-stranded form into a single-stranded form. Sequence verification takes place beyond the primer length (approx. 5-50 nucleotides of the synthesized segment) and thus differs from common amplification methods where only the primer sequence is responsible for sequence-specific binding.

A middle sequence segment (P1.1E3 and P2.1E3 FIGS. 10 and 13) of the synthesized fragment is not verified in an embodiment of controllers. While both controller oligonucleotides with synthesized strands have formed a double strand, the middle sequence segment of the synthesized strand thus remains in double-stranded form. The separation of this double-stranded segment (dissociation of the double-stranded form into single-stranded form) can be facilitated by the choice of temperature, for example. Specifically for very short lengths (of less than 10 nucleotides) of said fragment, strand separation takes place rapidly under reaction conditions (e.g. 55° C. or 65° C.).

Due to the interaction of the two controller oligonucleotides and the temperature-dependent separation of the middle segment, a permanent separation of the synthesized double strand into a single-stranded form takes place. The respective controller oligonucleotides remain on complementary parts of the respective strand and thus form a double strand. In a subsequent synthesis step, which takes place by the polymerase, the controller strands are displaced by the polymerase during the synthesis of a complementary strand, so that a new synthesized strand can be formed. The polymerases used are thus able to displace controller oligonucleotides from their binding of complementary segments of the fragment to be amplified.

Particularly the amplification takes place as exponential amplification, in which newly synthesized products of both primers (primer extension products) act as templates for further synthesis steps.

In said process, primer sequences are at least partially copied so that complementary primer binding sites are formed, which are present as sequence segments of a double strand immediately after their synthesis.

In the amplification method, synthesis steps of both strands and double-strand opening steps of the newly synthesized sequence segments take place in mutual alternation. Sufficient double-strand separation after synthesis is an important prerequisite for further synthesis. Overall, such an alternation of synthesis and double-strand separation steps can lead to exponential amplification.

In the amplification method according to the invention, the double-strand opening of main products of the amplification (amplification of nucleic acid chains comprising target sequence) takes place, among other things, by means of an oligonucleotide called controller oligonucleotide. The controller oligonucleotide comprises sequence segments that correspond to the respective target sequence.

According to the invention, strand separation is achieved by the use of controller oligonucleotides with predefined sequences, which, particularly by means of sequence-dependent nucleic acid-mediated strand displacement, separate a newly synthesized double strand consisting of both specific primer extension products. The resulting single-stranded segments of primer extension products comprise the target sequence as well as respective primer binding sites, which can serve as binding sites for further oligonucleotide primers, so that an exponential amplification of nucleic acid chains to be amplified is achieved. The primer extension reactions and strand displacement reactions take place simultaneously, particularly in the batch. The amplification takes place particularly under reaction conditions that do not allow a spontaneous separation of both specific synthesized primer extension products.

A specific exponential amplification of a target sequence comprising nucleic acid chain comprises a repetition of synthesis steps and double-strand opening steps (activation steps for primer binding sites) as a mandatory requirement for the amplification of the nucleic acid chain.

The opening of synthesized double strands is implemented as a reaction step, which is to be influenced sequence-specifically by the controller oligonucleotide. This opening can take place completely, up to the dissociation of both complementary primer extension products, or it can be partial.

According to the invention, the controller oligonucleotide comprises sequence segments that can interact with the target sequence and further sequence segments that cause, enable or promote said interaction. In the course of the interaction with the controller oligonucleotide, double-stranded segments of the synthesized primer extension products are converted into a single-stranded form via sequence-specific strand displacement. Said process is sequence-dependent: only when the sequence of the synthesized double strand comprises a certain degree of complementarity with the respective sequence of the controller oligonucleotide a sufficient opening of the double strand occurs, so that sequence segments essential for the continuation of the synthesis, e.g. primer binding sites, are converted into single stranded form, which corresponds to an “active state”. The controller oligonucleotide thus specifically “activates” the newly synthesized primer extension products, which comprise the target sequence, for further synthesis steps.

In contrast, sequence segments that do not comprise a target sequence are not converted into a single-stranded state and remain as double-stranded, which corresponds to an “inactive” state. The potential primer binding sites in such a double strand are disadvantaged or completely prevented from interacting with new primers, so that further synthesis steps generally do not take place on such “inactive” strands. Said missing or reduced activation (i.e. conversion into a single-stranded state) of synthesized nucleic acid strands after a synthesis step leads to the fact that in the subsequent synthesis step only a reduced quantity of primers can successfully participate in a primer extension reaction.

Due to an exponential amplification of main products (a nucleic acid chain to be amplified, which comprises target sequences), several synthesis steps and activation steps (double-strand opening steps) are combined to one amplification method and are carried out or repeated until the desired quantity of the specific nucleic acid chain is provided.

The reaction conditions (e.g. temperature) are designed in such away that spontaneous separation of complementary primer extension products is unlikely or significantly slowed down in the absence of a controller oligonucleotide.

The desired increase in the specificity of an amplification thus results from the sequence dependence of the separation of complementary primer extension products comprising a target sequence: the controller oligonucleotide enables or promotes said double-stranded separation as a result of the matching of its sequence segments with predetermined sequence segments of the primer extension products. This matching is verified by the controller oligonucleotide after each synthesis cycle. Exponential amplification results from successful repetitions of synthesis procedures and sequence-specific strand displacements by the controller oligonucleotide, i.e. “activations” (double-strand openings/double-strand separations/strand displacements resulting in single-stranded form of respective primer binding sites) of newly synthesized primer extension products.

The design of the controller oligonucleotide and the reaction conditions mean that the interaction of the controller oligonucleotide with non-specific primer extension products takes a different course. Due to insufficient complementarity between the controller oligonucleotide and a non-specific primer extension product, sufficient double strand separation/strand displacement of such a non-specific primer extension product from its template strand does not occur. The unspecific product thus remains predominantly in an “inactive”, double-stranded state.

The use of pre-defined controller oligonucleotide thus enables sequence-dependent verification of the contents of primer extension products between individual synthesis steps during exponential amplification and the generation of a selection or a selection of sequences for subsequent synthesis steps. A distinction can be made between “active” single-stranded states of newly synthesized specific primer extension products as a result of a successful interaction with a controller oligonucleotide, and “inactive” double-stranded states of newly synthesized non-specific primer extension products as a result of a deficient and/or insufficient and/or reduced and/or slowed down interaction with a controller oligonucleotide.

For exponential amplification, this results in the following effects: Under non-denaturing conditions, the separation of specifically synthesized strands takes place with the participation of controller oligonucleotide.

An exponential amplification of target sequence comprising nucleic acid chains takes place under sequence control (main reaction). Said sequence control takes place after each synthesis step and includes sequence segments that are located between primers and comprise a target sequence. The successful verification of the synthesis result after each synthesis step results in the separation of both specific primer extension products, which is the prerequisite for further specific synthesis steps.

During the amplification process, an initial formation/generation of unspecific primer extension products cannot be excluded in principle (by-products). Due to template-dependent synthesis, such unspecific primer extension products are generally present in double-stranded form immediately after their synthesis. However, the interaction with the controller oligonucleotide is either completely absent or limited, so that strand separation does not occur or the strand separation is slower than the main reaction. Thus, the transfer of faulty sequence information from one synthesis cycle to the next does not take place.

By selecting the reaction conditions and designing the controller oligonucleotide, it is therefore possible to exert a targeted influence on the efficiency of the regeneration of correct nucleic acid chain templates between individual synthesis steps within an amplification method. Generally, the higher the degree of matching of the synthesized sequence with the given sequence of the controller oligonucleotide, the more successful the separation of the synthesized products, the more successful the regeneration of correct templates from one synthesis step to the next. Conversely, a sequence deviation in by-products leads to an insufficient regeneration of template strands and thus to a slowing down of the synthesis initiation or to a reduction of the yield in each subsequent cycle. The overall exponential amplification of by-products either proceeds more slowly or does not take place at all and/or remains at an undetectable level.

Under reaction conditions that do not denature a double strand, the use of sequence-dependent nucleic acid-mediated strand displacement leads to sequence-specific separation of the two primer extension products during the amplification reaction in the method described: sufficient complementarity between newly synthesized extension fragments of the oligonucleotide primers with the sequence of a controller oligonucleotide specified at the beginning of an amplification is a prerequisite for successful strand displacement and can thus influence the efficiency of the strand separation of a double strand (consisting of the first and second primer extension product). Minor deviations result in a slowdown of strand displacement and thus also in a slowdown of strand separation. This can result in a slowing down of the overall reaction. With increasing difference in the sequence of the newly synthesized extension products and the sequence of the controller oligonucleotide given at the beginning of the reaction, the strand displacement is increasingly hindered, which in the end can no longer provide sufficient separation of both primer extension products. The two newly synthesized strands can no longer be sufficiently separated from each other, so that their binding sites are no longer accessible for oligonucleotide primers. This generally leads to the termination of an amplification of sequences with sequence deviations.

The execution of an amplification method using only one controller oligonucleotide has already been described in the PCT application PCT/EP2017/071011 and the European application 16185624.0. Although two different controller oligonucleotides are used in the present application, mechanisms of sequence-dependent strand displacement by a controller are very similar. For details on how to perform an amplification with only one controller, the skilled person is referred to said application.

The following are some advantageous embodiments for reaction conditions and individual components of the amplification reaction. Particularly, the structure of a primer (using the example of a first primer) as well as the structure of a corresponding controller oligonucleotide is explained.

### Embodiments for Polymerases:

The nucleic acid synthesis-inducing agent can be an enzyme-including compound or a system that acts to cause the synthesis of primer extension products.

Suitable enzymes for amplification for this purpose comprise e.g. DNA polymerases such as Bst polymerase and its modifications, Vent polymerase and other—particularly heat-stable—DNA polymerases, which enable the nucleotides to be incorporated in the correct manner, thereby forming the primer extension products complementary to each synthesized nucleic acid strand. Generally, synthesis is initiated at the 3′ end of each primer and then proceeds in the 5′ direction along the template strand until synthesis is complete or interrupted.

In certain embodiments, template-dependent DNA polymerases able to displace the strand are used in the first amplification. These include the large fragment of Bst polymerase or its modifications (e.g. Bst 2.0 DNA polymerase), Klenow fragment, Vent exo minus polymerase, Deepvent exo minus DNA polymerase, large fragment of Bsu DNA polymerase, large fragment of Bsm DNA polymerase.

In an embodiment, polymerases are used which do not comprise 5′-3′ exonuclease activity or do not comprise 5′-3′ FEN activity.

The deoxyribonucleoside triphosphates (dNTPs) dATP, dCTP, dGTP and TTP (or dUTP, or dUTP/TTP mixture) are added to the synthesis mixture in adequate quantities. In an embodiment, in addition to dNTPs, at least one further type of dNTP analogues can be added to the synthesis mixture. In an embodiment, these dNTP analogs comprise, for example, a characteristic label (e.g. biotin or fluorescent dye), so that if incorporated into a nucleic acid strand, said label is also integrated into the nucleic acid strand. In an embodiment other than dNTP, said dNTP analogues comprise at least one modification of the sugar-phosphate portion of the nucleotide, e.g. alpha-phosphorothioate-2′-deoxyribonucleoside triphosphates (or other modifications that confer nuclease resistance to a nucleic acid strand), 2′,3′-dideoxyribonucleoside triphosphates, acyclo-nucleoside triphosphates (or other modifications that terminate a synthesis). In a further embodiment, these dNTP analogs comprise at least one modification of a nucleobase, e.g. iso-cytosines, iso-guanosines (or other modifications of nucleobases of the extended genetic alphabet), 2-amino-adenosines, 2-thiouridines, inosines, 7-deazy-adenosines, 7-deaza-guanosines, 5-Me-cytosines, 5-propyl-urridines, 5-propyl-cytosines (or other modifications of nucleobases which can be incorporated by a polymerase in relation to natural nucleobases and lead to a change in strand stability). In a further embodiment, a dNTP analogue comprises both a modification of the nucleobase and a modification of the sugar-phosphate portion. In an embodiment, in addition to at least one natural dNTP substrate, at least one further type of dNTP analogue is added to the synthesis mixture.

### Strand Displacement (Strand Displacement):

A procedure which, by the action of a suitable agent, leads to the complete or partial separation of a first double strand (consisting, for example, of A1 and B1 strands) and to the simultaneous/parallel formation of a new second double strand, wherein at least one of the strands (A1 or B1) is involved in the formation of this new second strand. Two forms of strand displacement can be distinguished here.

In a first form of strand displacement, the formation of a new second double strand can take place using an already existing complementary strand, which is generally present in single-stranded form at the beginning of the reaction. In said case, the means of strand displacement (e.g. a preformed single-stranded strand C1, which comprises a sequence complementary to strand A1) acts on the first designed double strand (A1 and B1) and enters into a complementary bond with strand A1, thereby displacing strand B1 from the bond with strand A1. If the displacement of B1 is complete, the result of the C1 effect is a new double strand (A1:C1) and a single strand B1. If the displacement of B1 is incomplete, the result depends on several factors. For example, a complex of partially double-stranded A1:B1 and A1:C1 may be present as an intermediate product.

In a second form of strand displacement, the formation of a new second double strand can take place under a simultaneous enzymatic synthesis of the complementary strand, wherein one strand of the first preformed double strand acts as a template for the synthesis by the polymerase. The means of strand displacement (e.g. a polymerase with a strand displacement ability) acts on the first preformed double strand (A1 and B1) and synthesizes a new strand D1 complementary to strand A1, wherein strand B1 is simultaneously displaced from the bond with strand A1.

The term “nucleic acid-mediated strand displacement” summarizes a sum/series of intermediate steps which may be in equilibrium with each other and result in the temporary or permanent opening of a first preformed duplex (consisting of complementary strands A1 and B1) and formation of a new second duplex (consisting of complementary strands A1 and C1), wherein A1 and C1 are complementary to each other.

It is known that an essential structural requirement for initiating strand displacement is the establishment of spatial proximity between a duplex end (preformed first duplex of A1 and B1) and a single-stranded strand (C1) which initiates strand displacement (wherein A1 and C1 can form a complementary strand). Such proximity can be achieved particularly by means of a single stranded overhang (examples with short overhangs are known in the literature, called “Toehold”, see literature above), which temporarily or permanently binds the single stranded strand (C1) complementarily and thus brings complementary segments of strand C1 and A1 sufficiently close to each other so that a successful displacement of strand B1 can be initiated. The efficiency of initiating nucleic acid-mediated strand displacement is generally higher, the closer the complementary segments of strand A1 and C1 are positioned to each other.

Another essential structural prerequisite for the efficient continuation of nucleic acid-mediated strand displacement in inner segments is high complementarity between strands (e.g. between A1 and C1), which must form a new double strand. For example, single nucleotide mutations (in C1) can lead to the interruption of a strand displacement (described e.g. for branch migration).

The present invention makes use of the ability of complementary nucleic acids to form a sequence-dependent nucleic acid-mediated strand displacement.

Specific embodiments of the invention are explained in detail in the figures and examples.

### Embodiments for Reaction Conditions:

### Reaction Conditions for the First Amplification

Reaction conditions include buffer conditions, temperature conditions, reaction time and concentrations of the respective reaction components.

In the course of the reaction, the quantity of specifically produced nucleic acid to be amplified accumulates exponentially. The reaction comprising the synthesis of the extension products can be carried out to produce the desired quantity of the specific nucleic acid sequence as long as necessary. The method according to the invention is particularly continuous. In an embodiment, the amplification reaction is carried out at the same reaction temperature, wherein the temperature is particularly between 50° C. and 70° C. In an embodiment of the invention, the reaction temperature can also be variably controlled so that individual steps of the amplification process take place at different temperatures respectively.

The reagents required for exponential amplification are available in the same batch, particularly at the beginning of a reaction. In an embodiment of another method, reagents can also be added at later stages of the method.

In certain embodiments, no helicases or recombinases are used in the reaction mixture to separate the newly synthesized double strands of the nucleic acid to be amplified.

In certain embodiments, the reaction mixture does not contain biochemical energy-donating compounds such as ATP.

The quantity of the nucleic acid to be amplified at the beginning of the reaction can vary from a few copies to several billion copies in one batch. In diagnostic applications the quantity of the nucleic acid chain to be amplified can be unknown.

Other nucleic acids that are not to be amplified may also be present in the reaction. These nucleic acids may be derived from natural DNA or RNA or their equivalents. In an embodiment, control sequences are located in the same batch, which should also be amplified in parallel to the nucleic acid to be amplified.

In certain embodiments, a molar excess of approximately 10³:1 to approximately 10¹⁵:1 (ratio primer:template) of the primers and the controller oligonucleotide used is added to the reaction mixture, which comprises template strands for the synthesis of the nucleic acid chain to be amplified.

The quantity of the target nucleic acids may not be known when the method according to the invention is used in diagnostic applications, so that the relative amount of the primer and the controller oligonucleotide with respect to the complementary strand cannot be determined with certainty. The quantity of primer added will generally be in molar excess with respect to the quantity of the complementary strand (template) if the sequence to be amplified is contained in a mixture of complicated long chain nucleic acid strands. A large molar excess improves the efficiency of the method.

For example, the concentrations of primer-1, primer-2 and controller oligonucleotides used are in ranges between 0.01 μmol/l and 100 μmol/l, particularly between 0.1 μmol/l and 100 μmol/l, or between 0.1 μmol/l and 50 μmol/l, particularly between 0.1 μmol/l and 20 μmol/l. The high concentration of components can increase the speed of amplification. The respective concentrations of individual components can be varied independently to achieve the desired reaction result.

The concentration of polymerase is in ranges between 0.001 μmol/l and 50 μmol/l, particularly between 0.01 μmol/l and 20 μmol/l, particularly between 0.1 μmol/l and 10 μmol/l.

The concentration of individual dNTP substrates is in ranges between 10 μmol/l and 10 mmol/l, particularly between 50 μmol/l and 2 mmol/l, particularly between 100 μmol/l and 1 mmol/l. The concentration of dNTP can influence the concentration of divalent metal cations. If necessary, it is adjusted accordingly.

For example, Mg2+ is used as a divalent metal cation. The respective anion can be Cl, acetate, sulfate, glutamate, etc.

The concentration of divalent metal cations, for example, is adjusted to the optimal range for the respective polymerase and comprises regions between 0.1 mmol/l and 50 mmol/l, particularly between 0.5 mmol/l and 20 mmol/l, particularly between 1 mmol/l and 15 mmol/l.

Enzymatic synthesis generally takes place in a buffered aqueous solution. As buffer solutions, dissolved conventional buffer substances such as Tris-HCl, Tris-acetate, potassium glutamate, HEPES buffer, and/or sodium glutamate in common concentrations can be used. The pH value of these solutions is usually between 7 and 9.5, particularly around 8 to 8.5. The buffer conditions can be adjusted, for example, according to the recommendation of the manufacturer of the polymerase used.

Other substances, such as so-called Tm-depressors (e.g. DMSO, betaine, TPAC) etc. can be added to the buffer. Such substances reduce the melting temperature (“Tm depressors”) of double strands and can thus have a positive influence on the opening of double strands. Polymerase stabilizing components such as Tween 20 or Triton 100 can also be added to the buffer in usual quantities. EDTA or EGTA can be added in conventional quantities to complex heavy metals. Polymerase stabilizing substances, such as trehalose or PEG 6000 can also be added to the reaction mixture.

Particularly the reaction mixture does not contain any inhibitors of the strand displacement reaction and no inhibitors of a polymerase-dependent primer extension.

In an embodiment, the reaction mixture contains DNA-binding dyes, particularly intercalating dyes such as EvaGreen or SybrGreen. Such dyes may allow the detection of the formation of new nucleic acid chains.

The reaction mixture may also contain proteins or other substances, which for example originate from an original material and which particularly do not influence the amplification.

### Embodiments of Reaction Conditions

The temperature has an essential influence on the stability of the double strands.

In an embodiment, no temperature conditions are used during the amplification reaction, which essentially leads to the separation of double strands of the nucleic acid to be amplified in the absence of controller oligonucleotide. This is to ensure that the double-strand separation of nucleic acid chains to be amplified is dependent on the presence of the controller oligonucleotide throughout the amplification process.

At a temperature approximately equal to the measured melting temperature (Tm) of the nucleic acid to be amplified, a spontaneous separation of both strands of the nucleic acid to be amplified occurs, so that the influence of the controller oligonucleotide on the separation of synthesized strands and thus on the sequence specificity of the amplification is minimal to limited.

In exponential amplification, which is less sequence-specific (i.e. less controller oligonucleotide-dependent), the reaction temperature can be around the melting temperature (i.e. Tm plus/minus 3° to 5° C.) of the nucleic acid to be amplified. At such a temperature, sequence differences between the controller oligonucleotide and the synthesized primer extension product can generally be well tolerated in a strand displacement reaction.

Even in the temperature ranges from about (Tm minus 3° C.) to about (Tm minus 10° C.), spontaneous strand separation of synthesized primer extension products can still occur, although with lower efficiency. The influence of the controller oligonucleotide on the sequence specificity of the nucleic acid to be amplified is higher than at temperature conditions around the melting temperature (Tm) of the nucleic acid chain to be amplified.

Although strand separation occurs with decreasing reaction temperature essentially due to the interaction of the newly synthesized duplex with the controller oligonucleotide, duplexes from primers can also spontaneously disintegrate at the extension temperature under the above-mentioned conditions, i.e. without sequence-dependent strand displacement by the controller oligonucleotide. For example, the reaction temperature for reduced sequence-specific amplification is in ranges between approx. (Tm minus 3° C.) and approx. (Tm minus 10° C.), particularly between approx. (Tm minus 5° C.) and approx. (Tm minus 10° C.). At such temperatures, sequence differences between the controller oligonucleotide and the synthesized primer extension product are less tolerable in a strand displacement reaction.

A high sequence specificity of the amplification of the method is mainly achieved if the newly synthesized strands of the nucleic acid to be amplified cannot dissociate spontaneously into single strands under reaction conditions. In such a case, the sequence-specific strand displacement by the controller oligonucleotide plays a decisive role in sequence-specific strand separation and is largely responsible for the sequence specificity of the amplification reaction. This can generally be achieved if the reaction temperature is significantly below the melting temperature of both strands of the nucleic acid to be amplified and no other components are used for a strand separation, for example no helicases or recombinases. For example, in sequence-specific amplification the reaction temperature is in ranges between approx. (Tm minus 10° C.) and approx. (Tm minus 50° C.), particularly between approx. (Tm minus 15° C.) and approx. (Tm minus 40° C.), particularly between approx. (Tm minus 15° C.) and approx. (Tm minus 30° C.).

In an embodiment of amplification, the maximum reaction temperature during the entire amplification reaction is not increased above the melting temperature of the nucleic acid chain to be amplified.

In a further embodiment of the amplification, the reaction temperature can be increased at least once above the melting temperature of the nucleic acid chains to be amplified. The increase in temperature can, for example, take place at the beginning of the amplification reaction and lead to denaturation of double strands of a genomic DNA. It should be noted that during such a step the dependence of the double-strand separation on the action of the controller oligonucleotide is eliminated or at least significantly reduced.

The reaction temperatures of the individual steps of the amplification reaction can be in the ranges from about 15° C. to about 85° C., particularly in the ranges from about 15° C. to about 75° C., particularly in the ranges from about 25° C. to about 70° C.

Generally, the reaction temperature can be optimally adjusted for each individual reaction step, so that such a temperature is achieved for each reaction step. The amplification reaction thus comprises a repetitive change of temperatures which are repeated cyclically. In an advantageous embodiment of the method, reaction conditions for several reaction steps are standardized so that the number of temperature steps is less than the number of reaction steps. In such an embodiment of the invention, at least one of the steps of the amplification takes place at a reaction temperature which is different from the reaction temperature of other steps of the amplification. Thus, the reaction does not proceed isothermally, but the reaction temperature is cyclically changed.

For example, at least two temperature ranges are used during amplification, which are induced alternately (cyclic change of temperatures between individual temperature ranges). In an embodiment, the lower temperature range comprises for example temperatures between 25° C. and 60° C., particularly between 35° C. and 60° C., particularly between 50° C. and 60° C., and the upper temperature region comprises for example temperatures between 60° C. and 75° C., particularly between 60° C. and 70° C.

In an embodiment, the lower temperature range comprises for example temperatures between 15° C. and 50° C., particularly between 25° C. and 50° C., particularly between 30° C. and 50° C., and the upper temperature region comprises for example temperatures between 50° C. and 75° C., particularly between 50° C. and 65° C.

In an embodiment, the lower temperature range comprises for example temperatures between 15° C. and 40° C., particularly between 25° C. and 40° C., particularly between 30° C. and 40° C., and the upper temperature range comprises for example temperatures between 40° C. and 75° C., particularly between 40° C. and 65° C.

The temperature can be kept constant in the respective ranges or changed as a temperature gradient (falling or rising).

Further explanations of temperature settings are given in detail in the following segments for embodiments.

Each induced temperature can be maintained for a certain time, resulting in an incubation step. The reaction mixture can thus be incubated for a certain time during amplification at a selected temperature. Said time can be different for each incubation step and can depend on the progress of the respective reaction at a given temperature (e.g. primer extension or strand displacement etc.). The time of an incubation step can comprise the following ranges: between 0.1 sec and 10,000 sec, particularly between 0.1 sec and 1000 sec, particularly between 1 sec and 300 sec, particularly between 1 sec and 100 sec.

By changing the temperature in this way, individual reaction steps can be carried out, particularly at a selected temperature. Thus, yields of a respective reaction step can be improved. Within a synthesis cycle a temperature change or a temperature change between regions can be performed several times. A synthesis cycle can therefore comprise at least one temperature change. Such a temperature change can, for example, be executed routinely in a PCR device/thermal cycler as a temporal program.

An embodiment refers to an amplification method in which at least one of the steps comprising strand displacement and at least one of the steps comprising primer extension reactions take place simultaneously or in parallel and under identical reaction conditions. In such an embodiment, for example, a primer extension reaction of at least one oligonucleotide primer (e.g. of the first oligonucleotide primer) can take place particularly under temperature conditions in the lower temperature ranges. On the other hand, strand displacement takes place with the participation of controller oligonucleotide and a further primer extension reaction (e.g. from the second oligonucleotide primer) particularly in the reaction step in the upper temperature ranges.

A further embodiment relates to an amplification method in which at least one of the steps comprising strand displacement by the controller oligonucleotide and at least one of the steps comprising the primer extension reaction are performed at different temperatures. In such an embodiment, primer extension reactions of at least one oligonucleotide primer (e.g. of the first oligonucleotide and/or of the second oligonucleotide primer) can, for example, take place particularly under temperature conditions in the lower temperature ranges. In contrast, strand displacement takes place with the participation of controller oligonucleotide, particularly in the reaction step in the upper temperature ranges.

In a further embodiment, all steps of an amplification reaction take place under the same reaction conditions.

In such an embodiment, the amplification method can be carried out under isothermal conditions, i.e. no temperature changes are required to carry out the method. In such an embodiment of the invention, the entire amplification reaction takes place under constant temperature, i.e. the reaction is isothermal. The time of such a reaction comprises for example the following ranges: between 100 sec and 30,000 sec, particularly between 100 sec and 10,000 sec, particularly between 100 sec and 1000 sec.

In the section “Examples” it is shown that it is possible to match the structures of individual reaction components and respective reaction steps in such way that an isothermal reaction is possible.

The sum of all method steps, which leads to a doubling of the quantity of a nucleic acid chain to be amplified, can be called synthesis cycle. Such a cycle can run respectively isothermally or be characterized by changes in temperature during its course. The temperature changes can be repeated from cycle to cycle and can be made identical.

Particularly advantageous are amplification methods in which the maximum achievable temperature only allows strand separation with participation of controller oligonucleotide essentially complementary if more than 5 nucleotides of the third region of the controller oligonucleotide can undergo binding of a controller oligonucleotide with the first primer extension product, particularly if more than 10, particularly if more than 20 nucleotides of the controller oligonucleotide undergo binding with the first primer extension product. Generally, the longer the required binding of a controller oligonucleotide to the complementary strand of the first primer extension product before the synthesized strands dissociate under reaction conditions, the more specific the amplification reaction will be. In detail, the desired degree of specificity can be determined by extending or shortening the third segment of the controller oligonucleotide.

A method step can take place at a constant temperature for the entire duration of the method or it can be repeated at different temperatures.

Individual method steps can be carried out one after the other respectively by adding individual components. In an advantageous embodiment, all reaction components necessary for the amplification process are located in a reaction mixture at the beginning of the amplification process.

The start of an amplification reaction can take place by adding a component, e.g. by adding a nucleic acid chain comprising a target sequence (e.g. a start nucleic acid chain), or a polymerase or divalent metal ions, or by creating reaction conditions necessary for the amplification, e.g. setting a required reaction temperature for one or more method steps.

The amplification can be carried out until the desired quantity of nucleic acid to be amplified is reached. In another embodiment, the amplification reaction is carried out for a time which would have been sufficient to obtain a sufficient quantity in the presence of a nucleic acid to be amplified. In an embodiment, the amplification reaction is carried out over a sufficient number of synthesis cycles (doubling times), which, in the presence of a nucleic acid to be amplified, would have been sufficient to obtain a sufficient quantity.

The reaction can be stopped by various interventions. For example, by changing the temperature (e.g. cooling or heating, wherein the polymerase is disturbed in its function) or by adding a substance that stops a polymerase reaction, e.g. EDTA or formamide.

After amplification, the amplified nucleic acid chain can be used for further analyses. Synthesized nucleic acid chains can be analyzed by different detection methods. For example, fluorescence-labeled oligonucleotide probes can be used or sequencing methods (Sanger sequencing or next-generation sequencing), solid-phase analyses such as microarray or bead array analyses, etc. The synthesized nucleic acid chain can be used as substrate/template in further primer extension reactions.

In an advantageous embodiment, the progress of the synthesis reaction is monitored during the reaction. This can be taken place, for example, by using intercalating dyes, e.g. Sybrgreen or Evagreen, or by using labeled primers (e.g. Lux-primer or Scorpion-primer) or by using fluorescence labeled oligonucleotide probes.

The detection of the change in fluorescence during amplification is implemented in a detection step of the method. The temperature and duration of this step can be adjusted to the respective requirements of an oligonucleotide probe. For example, the temperatures of the detection step comprise ranges between 20° C. and 75° C., particularly between 40 and 70° C., particularly between 55 and 70° C.

During the detection step, the illumination of the reaction takes place with light of a wavelength that is capable of exciting a used fluorophore of the detection system (a donor or a fluorescence reporter). Signal acquisition generally takes place in parallel to the excitation, wherein the specific fluorescence signal is detected and its intensity is quantified.

The structure of an oligonucleotide primer, which can specifically bind to the corresponding controller oligonucleotide, is explained using the example of the first oligonucleotide primer. The structure of the second oligonucleotide primer generally follows the same rules. The pair comprising the first oligonucleotide primer and the first controller oligonucleotide is also used to explain interactions between these and other components.

The primer structure for the first and second oligonucleotide primers is shown in detail using the first oligonucleotide primer as an example. The structure of the second oligonucleotide primer is similar to that of the first one; differences between the first and second primers can be depending on the embodiment, for example, in the sequence composition, the used lengths of the respective regions, as well as nucleotide modifications.

The first oligonucleotide primer (primer-1) is a nucleic acid chain which includes at least the following regions:
A first primer region in the 3′ segment of the first oligonucleotide primer which can bind to a strand of a nucleic acid chain to be amplified essentially sequence-specifically
A second region coupled directly or via a linker to the 5′-end of the first region of the first oligonucleotide primer, comprising a polynucleotide tail suitable for binding of a controller oligonucleotide and supporting strand displacement by the controller oligonucleotide, wherein the polynucleotide tail remains essentially single-stranded under reaction conditions, i.e. does not form stable hairpin structure or ds structures, and especially is not copied by the polymerase.

The total length of the first oligonucleotide primer is between 10 and 80, particularly between 15 and 50, particularly between 20 and 30 nucleotides or their equivalents (e.g. nucleotide modifications). The structure of the first oligonucleotide primer is adapted in such a way that it can undergo reversible binding of a controller oligonucleotide under selected reaction conditions. Furthermore, the structure of the first oligonucleotide primer is adapted to its primer function. Furthermore, the structure is adapted in such a way that strand displacement can be carried out using the controller oligonucleotide. In total, the structures of the first and second regions are adapted to each other so that exponential amplification can be performed.

In an advantageous embodiment of the invention, the first and second regions of the primer are coupled in a conventional 5′-3′ arrangement. In a further embodiment of the invention, the coupling of both segments takes place via a 5′-5′ bond, so that the second region has a reverse direction to the first region.

The coupling of regions between/under each other takes place particularly covalently. In an embodiment, the coupling between the first and second regions is a 5′-3′-phosphodiester coupling, which is conventional for DNA. In a further embodiment it is a 5′-5′-phosphodiester coupling. In a further embodiment it is a 5′-3′-phosphodiester coupling, wherein at least one linker (e.g. a C3, C6, C12 or HEG linker or an abasic modification) is positioned between adjacent terminal nucleotides or nucleotide modifications of the two regions.

Individual regions can include different nucleotide modifications. Individual elements of nucleotides can be modified: Nucleobase and backbone (sugar portion and/or phosphate portion).

Furthermore, modifications can be used which are missing or modified in at least one component of the standard nucleotide building blocks, e.g. PNA.

In a further embodiment, a second region of the first region of the oligonucleotide primer comprises additional sequences that do not bind to the controller oligonucleotide. Said sequences can be used for other purposes, such as binding to the solid phase. These sequences are particularly located at the 5′ end of the polynucleotide tail.

In a further embodiment, a first oligonucleotide primer may comprise a characteristic label. Examples for such a label are dyes (e.g. FAM, TAMRA, Cy3, Alexa 488 etc.) or biotin or other groups that can be specifically bound, e.g. digoxigenin.

### The First Region of the First Oligonucleotide Primer

The sequence length is between about 3-30 nucleotides, particularly between 5 and 20 nucleotides, wherein the sequence is predominantly complementary to the 3′-segment of one strand of the nucleic acid chain to be amplified. Specifically, said regions must be capable of specifically binding to the complementary 3′ segment of a second primer extension product. It should be possible to copy said first region during reverse synthesis and it also serves as template for the second strand. The nucleotide building blocks are particularly linked to each other via the usual 5′-3′ phosphodieser bond or phosphothioester bond.

The first primer region includes particularly those nucleotide monomers which do not or only insignificantly influence the function of the polymerase, for example:
natural nucleotides (dA, dT, dC, dG etc.) or their modifications without altered base pairing
modified nucleotides, 2-amino-dA, 2-thio-dT or other nucleotide modifications with different base pairing.

In an embodiment, the 3′-OH end of said region is particularly free of modifications and has a functional 3′-OH group which can be recognized by the polymerase. The first region of the primer is the initiator of the synthesis of the first primer extension product in the amplification process. In a further embodiment, the first region comprises at least one phosphorothioate compound, so that no degradation of the 3′-end of the primer by the 3′-exonuclease activity of polymerases can take place.

The sequence of the first region of the first oligonucleotide primer and the sequence of the second region of the controller oligonucleotide are particularly complementary to each other.

In an embodiment, the first primer region or its 3′ segment can bind to sequence segments of a target sequence.

### The Second Region of the First Oligonucleotide Primer

The second region of the first oligonucleotide primer is particularly a nucleic acid sequence which comprises at least one polynucleotide tail which, particularly during the synthesis reaction, remains uncopied by the polymerase and which is capable of binding with the first region of the controller oligonucleotide. The segment of the second region, which predominantly undergoes this binding of a controller oligonucleotide, can be referred to as polynucleotide tail.

Furthermore, the second region of the first oligonucleotide primer must not only specifically bind the controller oligonucleotide under reaction conditions, but also participate in the procedure of strand displacement by means of controller oligonucleotide. Consequently, the structure of the second region must be suitable for inducing a spatial proximity between the controller oligonucleotide and the respective double strand end (in detail, the 3′ end of the second primer extension product).

The design of the structure of the second region of the first oligonucleotide primer is described in more detail in several embodiments. The array of the oligonucleotide segments and the modifications used are taken into account, which lead to a stop in the polymerase-catalyzed synthesis.

The length of the second region is between 3 and 60, particularly between 5 and 40, particularly between 6 and 15 nucleotides or their equivalents.

The sequence of the second region can be arbitrary. Particularly it is not complementary with the nucleic acid to be amplified and/or with the second oligonucleotide primer and/or with the first region of the first segment of the first oligonucleotide primer. Furthermore, it does not contain any self-complementary segments, such as hairpins or stem loops.

The sequence of the second region is particularly matched to the sequence of the first region of a controller oligonucleotide, so that both sequences can undergo binding under reaction conditions.

In an embodiment, said binding is reversible under reaction conditions: there is thus a balance between bound and unbound components.

In particular, the sequence of the second region of the first oligonucleotide primer is chosen in such a way that the number of complementary bases that can bind to the first region of the controller oligonucleotide is between 1 and 40, particularly between 3 and 20, particularly between 6 and 15.

The function of the second region is, among other things, the binding of a controller oligonucleotide. In an embodiment, this binding is particularly specific, so that a second region of a first segment of a first oligonucleotide primer can bind a specific controller oligonucleotide. In an embodiment other than this, a second region can bind more than one controller oligonucleotide under reaction conditions.

There is generally no need for a perfect matching in sequence between the second region of the first oligonucleotide primer and the first segment of the controller oligonucleotide. The extent of complementarity between the second region of the first oligonucleotide primer and the first region of the controller oligonucleotide can be between 20% and 100%, particularly between 50% and 100%, particularly between 80% and 100%. The respectively complementary regions can be positioned immediately adjacent to each other or comprise non-complementary sequence segments in between.

The second region of the first oligonucleotide primer can include at least one Tm-modifying modification in an embodiment. By introducing such modifications, the stability of the binding between the second region of the first oligonucleotide primer and the first region of the controller oligonucleotide can be modified. For example, Tm-increasing modifications (nucleotide modifications or non-nucleotide modifications) can be used, such as LNA nucleotides, 2-amino adenosines or MGB modifications. On the other hand, Tm-lowering modifications can also be used, such as inosine nucleotides. Linkers (e.g. C3, C6, HEG-linkers) can also be integrated into the structure of the second region.

For strand displacement, the controller oligonucleotide must be placed in close proximity to the double strand end of the nucleic acid to be amplified. This double-strand end consists of segments of the first region of the first primer extension product and a respective complementary 3′ segment of the second primer extension product.

The polynucleotide tail predominantly binds complementarily to the controller oligonucleotide under reaction conditions and thus causes a temporary approximation of the second region of the controller oligonucleotide and the first region of a controller oligonucleotide extension product, so that a complementary binding between these elements can be initiated in a strand displacement process.

In an embodiment, binding of a controller oligonucleotide to the polynucleotide tail of the first oligonucleotide primer results in immediate contact. This means that the polynucleotide tail and the first region of the first oligonucleotide primer must be coupled to each other immediately. Thanks to such an arrangement, after binding of a controller oligonucleotide in its first region, there can be immediate contact between complementary bases of the second region of the controller oligonucleotide and respective bases of the first primer region, so that strand displacement can be initiated.

In a further embodiment, between structures of the polynucleotide tail and the first primer region, other structures of the second region of the first oligonucleotide primer are located. Thus, after binding of a controller oligonucleotide to the polynucleotide tail, the tail is not positioned immediate to the first region of the primer, but at a certain distance from it. The structures between the uncopyable polynucleotide tail and the copyable first regions of the oligonucleotide primer can generate such a distance. This distance has a value which is between 0.1 and 20 nm, particularly between 0.1 and 5 nm, particularly between 0.1 and 1 nm.

Such structures represent linkers (e.g. C3 or C6 or HEG linkers) or segments that are not complementary to the controller oligonucleotide (e.g. in the form of non-complementary, non-copyable nucleotide modifications). The length of these structures can generally be measured in chain atoms. Said length is between 1 and 200 atoms, particularly between 1 and 50 chain atoms, particularly between 1 and 10 chain atoms.

In order that the polynucleotide tail remains uncopyable by the polymerase under amplification conditions, the second region of the first oligonucleotide primer generally comprises sequence arrangements or structures that cause the polymerase to stop in the synthesis of the second primer extension product after the polymerase has successfully copied the first primer region. Said structures are designed to prevent the copying of the polynucleotide tail of the second region. Thus, the polynucleotide tail remains uncopied, particularly by the polymerase.

In an embodiment such structures are located between the first region and the polynucleotide tail.

In a further embodiment, the sequence of the polynucleotide tail can include nucleotide modifications that lead to the stopping of the polymerase. Thus, a sequence segment of the second region of the first region of the oligonucleotide primer can comprise both functions: it is a polynucleotide tail as well as a sequence of nucleotide modifications leading to the stop of the polymerase.

Modifications in the second region of the first oligonucleotide primers, which lead to a synthesis stop and thus leave the polynucleotide tail uncopyable, are summarized in this application under the term “first blocking unit or a stop region”.

Below are further embodiments of structures, which can lead to a stop in the synthesis of the second strand.

Several building blocks in oligonucleotide synthesis are known to prevent the polymerase from reading off the template and to lead to termination of polymerase synthesis. For example, non-copyable nucleotide modifications or non-nucleotide modifications are known. There are also synthesis types/arrays of nucleotide monomers within an oligonucleotide which lead to the termination of the polymerase (e.g. 5′-5 arrangement or 3′-3′ arrangement). Oligonucleotide primers with a non-copyable polynucleotide tail are also known in the state of the art (e.g. Scorpion primer structures or primers for binding to the solid phase). Both primer variants describe oligonucleotide primer structures which are able to initiate the synthesis of a strand on the one hand, so that a primer extension reaction can take place. The result is a first strand, which also integrates the primer structure with tail in the primer extension product. In the synthesis of a complementary strand to the first primer extension product, e.g. in a PCR reaction, the second strand is extended to the “blocking unit/stop structure” of the primer structure. Both described primer structures are designed in such way that the 5′ portion of the oligonucleotide primer remains single-stranded and is not copied by the polymerase.

In a further embodiment, the second region of the oligonucleotide primer comprises a polynucleotide tail, which comprises a conventional 5′- to 3′-arrangement along its entire length and includes non-copyable nucleotide modifications. Such non-copyable nucleotide modifications include 2′-O-alkyl RNA modifications, PNA, and morpholino. These modifications can be distributed differently in the second region of the primer.

The portion of non-copyable nucleotide modifications in the polynucleotide tail can be between 20% and 100%, particularly more than 50% of the nucleotide building blocks. Particularly, these nucleotide modifications are located in the 3′ segment of the second segment and thus border the first region of the first oligonucleotide primer.

In an embodiment, the sequence of non-copyable nucleotide modifications is at least partially complementary to the sequence in the template strand, so that primer binding to the template takes place involving at least part of these nucleotide modifications. In an embodiment, the sequence of non-copyable nucleotide modifications is not complementary to the sequence in the template strand.

The non-copyable nucleotide modifications are particularly covalently coupled to each other and thus represent a segment of the sequence in the second region. The length of said segment comprises between 1 and 40, particularly between 1 and 20 nucleotide modifications, particularly between 3 and 10 nucleotide modifications.

In an embodiment, the second region of the first oligonucleotide primer comprises a polynucleotide tail, which comprises a conventional 5′-3′ arrangement along its entire length and includes non-copyable nucleotide modifications (e.g. 2′-O-alkyl modifications) and at least one non-nucleotide linker (e.g. C3, C6, HEG linker). A non-nucleotide linker has the function of covalently connecting adjacent nucleotides or nucleotide modifications and at the same time interrupting the synthesis function of the polymerase in a site-specific manner.

Such a non-nucleotide linker should not separate the structures of the polynucleotide tail and the first region of the primer too far from each other. Rather, the polynucleotide tail should be in close proximity to the first region segment of the primer. A non-nucleotide linker is defined as a modification that is not longer than 200 chain atoms, especially not longer than 50 chain atoms, particularly not longer than 10 chain atoms. The minimum length of such a linker can be one atom. Examples of such non-nucleotide linkers are straight or branched alkyl linkers with an alkyl chain including at least one carbon atom, particularly at least 2 to 30, particularly 4 to 18. Such linkers are well known in oligonucleotide chemistry (e.g. C3, C6 or C12 linkers) and can be introduced during solid phase synthesis of oligonucleotides between the sequence of the polynucleotide tail and the sequence of the first region of the first oligonucleotide primer. Linear or branched polyethylene glycol derivatives are another example of such non-nucleotide linkers. A well-known example in oligonucleotide chemistry is hexaethylene glycol (HEG). Another example of such non-nucleotide linkers are abasic modifications (e.g. THE modification, as an analog of D-ribose).

If one or more of such modifications are integrated into a second region, they can effectively interfere with a polymerase in its copy function during its synthesis of the second primer extension product, so that segments remain uncopied after such a modification. The number of such modifications in the second region can be between 1 and 100, particularly between 1 and 10, particularly between 1 and 3.

The position of such a non-nucleotide linker can be located at the 3′ end of the second region and thus represent the transition from the first region to the second region of the oligonucleotide primer.

The position of the non-nucleotide linker in the middle segment of the second region can also be used. This will divide a polynucleotide tail into at least two segments. In an embodiment like this, the 3′ segment of the polynucleotide tail includes at least one, particularly several, e.g. between 2 and 20, particularly between 2 and 10 non-copyable nucleotide modifications. Said non-copyable nucleotide modifications are particularly located at the transition between the first and second regions of the oligonucleotide primer.

In a further embodiment, the second region of the oligonucleotide primers comprises a polynucleotide tail, which comprises a 5′-to-3′-arrangement along its entire length and includes at least one nucleotide monomer in an “inverted” 3′-to-5′-arrangement and which is positioned at the transition between the first and the second region of the first oligonucleotide primer.

In a further embodiment, the second region of the oligonucleotide primer comprises a polynucleotide tail, wherein such a polynucleotide tail consists entirely of nucleotides that are directly contiguous to the first region of the first oligonucleotide primer in an inverted arrangement such that coupling of the first and second regions takes place by 5′-5′ position. An advantage of such an arrangement is that after copying the first region, the polymerase encounters an “inverted” arrangement of nucleotides, which typically leads to termination of the synthesis at this position.

In the case of an “inverted” arrangement of nucleotides along the entire length of the polynucleotide tail, the 3′-terminal nucleotide of the polynucleotide tail should be blocked at its 3′-OH end particularly, in order to prevent side reactions. Alternatively, a terminal nucleotide that does not comprise a 3′-OH group at all, e.g. a dideoxy nucleotide, can be used.

In an embodiment of this kind, the respective nucleotide arrangement in the controller oligonucleotide should of course also be adapted. In such a case, the first and second regions of the controller oligonucleotide must be linked in 3′-3′ array.

In an further embodiment, the second region of the oligonucleotide primer comprises a polynucleotide tail, which comprises a conventional 5′-to-3′-array along its entire length and includes at least one nucleotide modification, which is not a complementary nucleobase for the polymerase when the synthesis is performed with exclusively natural dNTP (dATP, dCTP, dGTP, dTTP or dUTP).

For example, iso-dG or iso-dC nucleotide modifications can be integrated as single, but particularly several (at least 2 to 20) nucleotide modifications in the second region of the first oligonucleotide primer. Further examples of nucleobase modifications are various modifications of the extended genetic alphabet. Such nucleotide modifications particularly do not support complementary base pairing with natural nucleotides, so that a polymerase (at least theoretically) does not incorporate a nucleotide from the series (dATP, dCTP, dGTP, dTTP or dUTP). In reality, however, rudimentary incorporation may still occur, particularly at higher concentrations of dNTP substrates and prolonged incubation times (e.g. 60 min or longer). Therefore, particularly several such nucleotide modifications positioned at adjacent sites should be used. The stop of polymerase synthesis is caused by the lack of suitable complementary substrates for these modifications. Oligonucleotides with Iso-dC or Iso-dG can be synthesized by standard methods and are available from several commercial suppliers (e.g. Trilink-Technologies, Eurogentec, Biomers GmbH). Optionally, the sequence of the first region of the controller oligonucleotide can be adapted to the sequence of such a second region. In this case, complementary nucleobases of the extended genetic alphabet can be integrated into the first region of the controller oligonucleotide in the respective regions during chemical synthesis. For example, Iso-dG can be integrated in the second region of the first primer, its complementary nucleotide (Iso-dC-5-Me) can be placed at the appropriate position in the first region of the controller oligonucleotide.

In summary, the termination of the synthesis by the polymerase in the second region can be achieved in different ways. However, said termination takes place particularly after the polymerase has copied the first region of the first oligonucleotide primer. This ensures that a second primer extension product has a matching primer binding site in its 3′ segment. Said primer binding site is exposed during strand displacement and is thus available for rebinding another first oligonucleotide primer.

During the synthesis of the complementary strand to the first primer extension product, the primer extension reaction stops before the polynucleotide tail. Since this polynucleotide tail thus remains single-stranded for interaction with the controller oligonucleotide and is thus available for binding, it supports the initiation of the strand displacement reaction by the controller oligonucleotide by bringing the respective complementary segments of the controller oligonucleotide into immediate proximity with the matching duplex end. The distance between the complementary part of the controller oligonucleotide (second region) and the complementary part of the extended oligonucleotide primer (first region) is thereby minimized. Such spatial proximity facilitates the initiation of strand displacement.

In a schematic representation of a nucleic acid-mediated strand displacement reaction, a complementary sequence of controller oligonucleotide is now located immediate near the matching duplex end. Thereby, there is competition for binding to the first region of the first oligonucleotide primer between the strand of the controller oligonucleotide and the template strand complementary to the primer. The nucleic acid-mediated strand displacement procedure is initiated by repetitive closure and formation of base pairing between the primer regions and the complementary segment of the controller oligonucleotide (second region of the controller nucleotide) or the complementary segment of the template strand.

Generally, the closer the respective complementary sequence segment of the controller oligonucleotide is located to the complementary segment of the regions, the higher the yield of initiating strand displacement. On the other hand, increasing said distance decreases the yield of initiating strand displacement.

In the context of the present invention, it is not absolutely necessary that the initiation of strand displacement works with maximum yield. Therefore, distances between the 5′-segment of the first primer region of the first oligonucleotide primer, which undergoes binding with a complementary strand of the template and forms a complementary duplex, and a respective complementary sequence segment in the controller oligonucleotide when bound to the polynucleotide tail of the second region of the first oligonucleotide primer, can be in the following ranges: between 0.1 and 20 nm, particularly between 0.1 and 5 nm, particularly between 0.1 and 1 nm. In certain embodiments, said distance is less than 1 nm. Expressed in other units, said distance corresponds to a distance of less than 200 atoms, particularly less than 50 atoms, particularly less than 10 atoms. In certain embodiments, said distance is one atom. The information on distance is only for orientation and illustrates that shorter distances between these structures offer significant advantages. In many cases, measuring this distance is only possible by analyzing the exact structures of oligonucleotides and measuring sequence distances or linker lengths.

The first primer may also comprise further sequence segments that are not required for interaction with controller oligonucleotide or template strands. Such sequence segments can bind further oligonucleotides, which are used as detection probes or immobilization partners when binding to the solid phase.

### Primer Function of the First Oligonucleotide Primer

The first oligonucleotide primer can be used in several steps. Primarily, it takes over a primer function in the amplification process. The primer extension reaction is performed using the second primer extension product as a template. In a further embodiment, the first oligonucleotide primer can use the start nucleic acid chain as a template at the beginning of the amplification reaction. In a further embodiment, the first oligonucleotide primer can be used to create/provide a start nucleic acid chain.

During the amplification process, the first primer serves as an initiator for the synthesis of the first primer extension product using the second primer extension product as a template. The 3′ segment of the first primer comprises a sequence that can bind predominantly complementary to the second primer extension product. The enzymatic extension of the first oligonucleotide primer using the second primer extension product as template leads to the formation of the first primer extension product. Such first primer extension product comprises the target sequence or its sequence portions. In the course of the synthesis of the second primer extension product, the sequence of the copyable portion of the first oligonucleotide primer is recognized by the polymerase as template and a respective complementary sequence is synthesized, resulting in a respective primer binding site for the first oligonucleotide primer. The synthesis of the first primer extension product takes place up to and including the 5′ segment of the second oligonucleotide primer. Immediately after the synthesis of the first primer extension product, this product is bound to the second primer extension product and forms a double-stranded complex. The second primer extension product is displaced from this complex by the controller oligonucleotide in a sequence-specific manner. In the process, the controller oligonucleotide binds to the first primer extension product. After a successful strand displacement by the controller oligonucleotide, the second primer extension product can itself serve as a template for the synthesis of the first primer extension product. The now vacated 3′-segment of the first primer extension product can bind another second oligonucleotide primer, so that a new synthesis of the second primer extension product can be initiated.

Furthermore, the first oligonucleotide primer can serve as an initiator for the synthesis of the first oligonucleotide primer extension product starting from the start nucleic acid chain at the beginning of the amplification. In an embodiment, the sequence of the first primer is completely complementary to the respective sequence segment of a start nucleic acid chain. In an embodiment, the sequence of the first oligonucleotide primer is only partially complementary to the respective sequence segment of a start nucleic acid chain. However, this deviating complementarity should not prevent the first oligonucleotide primer from starting a predominantly sequence-specific primer extension reaction. The respective differences in the complementarity of the first oligonucleotide primer to the respective position in the start nucleic acid chain are particularly located in the 5′-segment of the first region of the first oligonucleotide primer, so that in the 3′-segment predominantly complementary base pairing and initiation of the synthesis is possible. For the initiation of the synthesis, for example, specifically the first 4-10 positions in the 3′ segment should be fully complementary to the template (start nucleic acid chain). The remaining nucleotide positions may differ from perfect complementarity. The degree of perfect complementarity in the remaining 5′-segment of the first region of the first oligonucleotide primers can thus comprise ranges between 50% and 100%, especially between 80% and 100% of the base composition. Depending on the length of the first region of the first oligonucleotide primers, comprising the sequence variations from 1 to a maximum of 15 positions, especially 1 to a maximum of 5 positions. Thus, the first oligonucleotide primer can initiate a synthesis from a start nucleic acid chain. In a subsequent synthesis of the second primer extension product, copyable sequence segments of the first oligonucleotide primer are copied by the polymerase, so that again in subsequent synthesis cycles a fully complementary primer binding site within the second primer extension product is designed for binding of the first oligonucleotide primer and is available in subsequent synthesis cycles.

In a further embodiment, the first oligonucleotide primer can be used in the preparation of a start nucleic acid chain. Such a first oligonucleotide primer can bind to a nucleic acid (e.g. a single-stranded genomic DNA or RNA or their equivalents comprising a target sequence) predominantly/particularly sequence-specific and initiate a template-dependent primer extension reaction in the presence of a polymerase. The binding position is chosen such that the primer extension product comprises a desired target sequence. The extension of the first oligonucleotide primer results in a nucleic acid strand which comprises a sequence complementary to the template. Such a strand can be detached from the template (e.g. by heat or alkali) and thus be converted into a single-stranded form. Such a single-stranded nucleic acid chain can serve as a start nucleic acid chain at the beginning of the amplification. Such a start nucleic acid chain comprises in its 5′-segment the sequence parts of the first oligonucleotide primer, further it comprises a target sequence or its equivalents and a primer binding site for the second oligonucleotide primer. Further steps are explained in the segment “Start nucleic acid chain”.

The synthesis of the first primer extension product is a primer extension reaction and forms a sub-step in the amplification process. The reaction conditions during this step are adjusted accordingly. The reaction temperature and the reaction time are chosen in such a way that the reaction can take place successfully. The respectively advantageous temperature in this step depends on the polymerase used and on the binding strength of the respective first oligonucleotide primer to its primer binding site and comprises for example ranges from 15° C. to 75° C., particularly from 20 to 65° C., particularly from 25° C. to 65° C. The concentration of the first oligonucleotide primer comprises ranges from 0.01 μmol/l to 50 μmol/l, particularly from 0.1 μmol/l to 20 μmol/l, particularly from 0.1 μmol/l to 10 μmol/l.

In an embodiment, all steps of the amplification process are performed under stringent conditions that prevent or slow down the formation of unspecific products/by-products. Such conditions include higher temperatures, for example above 50° C.

If more than one specific nucleic acid chain has to be amplified in one batch, in an embodiment especially sequence-specific oligonucleotide primers are used for the amplification of respective target sequences.

Particularly, sequences of the first and second oligonucleotide primer and the controller oligonucleotide are matched to each other in such a way that side reactions, e.g. primer-dimer formation, are minimized. For this purpose, for example, the sequences of the first and second oligonucleotide primer are matched to each other in such a way that both oligonucleotide primers are not able to start or support an amplification reaction in the absence of a suitable template and/or target sequence and/or start nucleic acid chain. This can be achieved for example by the fact that the second oligonucleotide primer does not comprise a primer binding site for the first oligonucleotide primer and the first oligonucleotide primer does not comprise a primer binding site for the second oligonucleotide primer. Furthermore, it should be avoided that the primer sequences comprise extended self-complementary structures.

In an embodiment, the synthesis of the first and second primer extension product is performed at the same temperature. In an embodiment the synthesis of the first and second primer extension products is performed at different temperatures. In a further embodiment, the synthesis of the first primer extension product and the strand displacement by the controller oligonucleotide takes place at the same temperature. In a further embodiment, the synthesis of the first primer extension product and the displacement of the strand by the controller oligonucleotide takes place at different temperatures.

### Oligonucleotide Primers Comprising Additional Sequence Segments:

The above-mentioned structures of the first primer and the second primer can be understood as so-called “basic structure of the primer” and “minimal structure of the primer”, respectively.

Such basic structures of oligonucleotides with primer function (e.g. first oligonucleotide primer, second oligonucleotide primer, possibly third oligonucleotide primer, possibly fourth oligonucleotide primer, etc.) comprise sequence segments which are advantageous for the execution of the amplification method, for example the first and second regions of the first primer.

Such a basic structure of the primers can be extended by further, additional sequence segments. Such additional sequence segments comprise structures which are not necessary for the execution of the amplification method, but can be useful for other objectives.

Such additional segments can optionally be introduced into a primer and used for further functions or reactions. Thus, the primer extension products synthesized by the polymerase (starting e.g. from the first and/or second primer) can be linked to such sequence segments. This allows the integration of such additional sequence segments and primer extension products into a molecular structure. Such an integration can be advantageous in certain embodiments. A variety of applications for primer sequences with additional sequence segments are known to a skilled person.

Several functions are known to a skilled person, which are supported by additional sequence segments of the primer.

For example, the introduction of additional structures can be used as a means to mediate intermolecular or intramolecular binding. Several examples of such structures are known to a skilled person. For example, probes can be designed according to such a principle of intramolecular binding, e.g. in Scorpion primers. Furthermore, sequence segments can be used to bind additional oligonucleotides. Sequence-specific intermolecular binding can be achieved by using stringent conditions. Such interactions can be used, for example, to bind amplification products to a solid phase by complementary binding to immobilized oligonucleotides.

Another example is the introduction of so-called adaptor sequences and/or the use of further segments for the unique coding or sequence-specific labeling of primers and primer extension products (so-called primer barcoding). This is used for example for NGS-Library Preparation (Ståhlberg et al Nucleic Acids Res. 2016 Jun. 20; 44(11): e105). During sequence analysis of primer extension products, such a label can be used to take place at a later time for sequence assignment.

Another example is the use of further sequence segments to introduce specific sequences with binding of certain proteins, e.g. restrictions endonucleases etc.

Another example is the use of further segments to introduce spacer sequences, which are not intended to bind a specific interaction partner, but rather to increase the distance between adjacent sequences.

Such additional sequences can either be positioned at the copyable portion of the primer or be added to the uncopyable portion of the primer. Several factors play a role in determining whether a sequence segment is copied or not. For example, positioning of the sequence segment in the respective oligonucleotide, used nucleotide modifications (e.g. C3, HEG, 2′-Ome etc.) can decide whether a sequence segment is used as a template during a process step or not.

In an embodiment, an additional sequence segment is introduced into copyable regions of the primer, e.g. at the 5′ segment of the copyable portion of the second primer, so that, for example, when the primer sequence is read off during a procedure to synthesize a target sequence, additional sequence segments are also read off by the polymerase. The length of such an additional sequence segment comprises ranges from 3 to 50 nucleotides. The composition of these sequence segments allows in an embodiment of this type the synthesis by a polymerase, said segment serves as a template for polymerase-dependent synthesis. In such a segment, natural nucleotides are used, e.g. dA, dG, dC, dT.

In a further embodiment, additional segments can be positioned at the 5′ terminus of the primer, which should not be copied during the synthesis of specific amplification fragments comprising a target sequence. This can be achieved, for example, by positioning one or more modifications or chemical groups that prevent the polymerase from synthesizing a complementary strand (e.g. HEG, C3, a segment comprising 4-10 nucleotides with 2′-Ome modifications, etc.). Such a modification may, for example, be positioned at the 5′ terminus of the copyable portion of the second primer and hinder the continuation of the synthesis. For example, a HEG group can be introduced at the 5′ end of the copyable segment of the second primer, and then an additional sequence segment can be introduced afterwards.

Furthermore, an additional sequence segment can be positioned at the 5′ terminus of the second region of the first primer. Such localization of additional segments prevents the synthesis of a complementary strand during a regular synthesis of specific amplification products comprising a target sequence.

The length of such an additional sequence segment comprises ranges from 3 to 50 nucleotides. The base composition may comprise natural nucleobases (A, G, C, T, U, inosines) or modifications at different positions of nucleotides (e.g. at the bases, such as 2-amino adenine, iso-guanine, iso-cytosines, 5-propargyl-uridines, 5-propargyl-cytosines or at the sugar-phosphate backbone, such as LNA, 2′-Ome, 2′-halogens etc.). In an embodiment, a first primer and additional sequence segments are combined to form an oligonucleotide. In a certain further embodiment, a second primer and additional sequence segments are combined to form an oligonucleotide.

Such additional sequence segments are designed in an oligonucleotide in such way that they do not prevent the amplification method of target sequences. This is achieved, for example, by avoiding or reducing inhibitory interactions with the structures of the primers or controllers essential for the method. In certain embodiments, additional structures can form complementary double stranded segments with other regions under the selected reaction conditions. However, particularly such double-stranded segments do not prevent a specific amplification of a target sequence. In certain embodiments, such additional segments do not interact or bind to the first or second region of the first primer. In certain embodiments, such additional segments do not interact with the controller oligonucleotide. In certain embodiments, such additional sequence segments do not interact with other primers in the reaction. In certain embodiments such additional sequence segments do not interact with P1.1-Ext or P2.1-Ext or other amplification fragments comprising a target sequence. In certain embodiments, such additional sequence segments do not form double-stranded segments with the first or second region of the first primer that are stable under reaction conditions and completely prevent the function of the first or second region.

In certain embodiments, such additional segments do not interact with or bind to the second primer. In certain embodiments, such additional sequence segments particularly do not interact with the 3′ segment of the second primer.

In certain embodiments, the first primer comprises an additional segment of the first primer (additional sequence variant P1) at its 5′ end of the second region. Said segment optionally comprises a sequence of 10-50 nucleotides, which does not interfere with the amplification method of target sequences (e.g. does not form secondary structures with primers). Furthermore, said segment optionally comprises a sequence of about 5 to 15 nucleotides of the copyable first region of the first primer. The additional sequence variant P1 comprises natural nucleotides as monomers (A, C, G, T) and can potentially serve as a template for a polymerase.

In certain embodiments the second primer comprises at its 5′ terminus an additional segment of the second primer (additional sequence variant P2). Optionally, said segment comprises a sequence of 10-50 nucleotides which does not interfere with the amplification method of target sequences (e.g. does not form secondary structures with primers). Furthermore, said segment optionally comprises a sequence of about 5 to 15 nucleotides of the copyable region of the second primer. The additional sequence variant P2 comprises natural nucleotides as monomers (A, C, G, T) and can potentially serve as a template for a polymerase.

It has been observed that such oligonucleotides comprising a first primer and additional sequence variant P1 or oligonucleotides comprising a second primer and additional sequence variant P2 are less susceptible to side reactions than oligonucleotides comprising only a first primer or oligonucleotides comprising only a second primer. In certain embodiments, for example, the generation and/or amplification of unspecific primer-dimer structures can be delayed. In such a side reaction, the formation of by-products comprising no target sequence can be optionally reduced or delayed. Thus, for example, the premature consumption of primers can be reduced or delayed. The use of such oligonucleotides is advantageous, for example, if primer-dimers comprising first primers (PD P1) or primer-dimers comprising second primers (PD P2) are generated by side reactions and lead to premature consumption of primers in the reaction. The use of primers with such additional structures (first primer with additional sequence variant P1 and/or second primer with additional sequence variant P2) is advantageous in certain embodiments when non-specific reactions are observed in an amplification reaction. Such side reactions can be favored by several factors, including:
longer reaction times (e.g. in ranges between 1 hr and 100 hr)
higher concentrations of primers are used (e.g. in ranges between 1 μmol/l and 1 mmol/l)
higher concentrations of polymerase are used (e.g. in ranges above 10 unit/10 μl)
Multiplex reactions (e.g. amplification of more than 10 different target sequences in one batch).
high concentrations of complex nucleic acid chains in the batch (e.g. concentrations above 1 μg hgDNA in 50 μl)

Individual factors alone or in combination with other factors can promote side reactions.

Generally, side reactions (e.g. non-specific primer-dimer formation) can be prevented by optimizing reaction components and/or reaction conditions, e.g. by reducing concentrations of individual components, shorter reaction times, sequence design of primer sequences, selection of more stringent reaction conditions. The additional sequence segments listed in an advantageous embodiment (oligonucleotides comprising a first primer and additional sequence variant P1 or oligonucleotides comprising a second primer and additional sequence variant P2) represent a further possible way to delay certain side reactions.

In exemplary embodiments, oligonucleotide primers with additional sequence segments are shown. In said exemplary embodiments additional sequence segments are used, which do not participate in the specific amplification of a target sequence and contribute to delay side reactions. Thus, oligonucleotides comprising a first primer and additional sequence variant P1 and oligonucleotides comprising a second primer and additional sequence variant P2 are used.

A skilled person will recognize that an oligonucleotide primer structure, which is advantageous for the specific amplification of a target sequence (this structure can also be called “basic structure” or “minimal structure”), can also comprise further, additional sequence segments (e.g. additional sequence variant P1 or additional sequence variant P2). Such additional sequence segments can provide a variety of other advantageous or useful properties or functions.

### Certain Embodiments of the Controller Oligonucleotide

The structure and function of the controller oligonucleotide is explained using the example of the first controller oligonucleotide. The structure of the second controller oligonucleotide generally follows the same rules. Depending on the embodiment, the differences between the first and the second controller can be in an embodiment, for example, in the concrete composition of the sequences, the lengths of the respective regions used, as well as nucleotide modifications.

A controller oligonucleotide comprises:
A first single-stranded region which can bind to the polynucleotide tail of the second region of the first oligonucleotide primer,
a second single-stranded region which can bind essentially complementary to the first region of the first oligonucleotide primer,
a third single-stranded region, which is essentially complementary to at least one segment of the extension product of the first primer extension product,
   and the controller oligonucleotide does not serve as a template for primer extension of the first or second oligonucleotide primer.

Generally, the sequence of the third region of the controller oligonucleotide is matched to the sequence of the nucleic acid to be amplified, since this is the template for the sequence of nucleotides in the extension product of a first primer. The sequence of the second region of the controller oligonucleotide is matched to the sequence of the first region. The structure of the first region of the controller oligonucleotide is adapted to the sequence of the second region of the first oligonucleotide primer, mainly to the structure of the polynucleotide tail.

A controller oligonucleotide can also include further segments which do not belong to the first, second or third region. These sequences can, for example, be attached as flanking sequences at the 3′ and 5′ end. Particularly said sequence segments do not interfere with the function of the controller oligonucleotide.

The structure of the controller oligonucleotide particularly comprises the following properties:

The individual regions are covalently bound to each other. The binding can take place via conventional 5′-3′ binding, for example. For example, a phospho-diester bond or a nuclease-resistant phospho-thioester bond can be used.

A controller oligonucleotide can bind to the polynucleotide tail of the first oligonucleotide primer by means of its first region, wherein binding is mediated primarily by hybridization of complementary bases. The length of said first region is 3-80 nucleotides, particularly 4-40 nucleotides, particularly 6-20 nucleotides. The degree of sequence concordance between the sequence of the first region of the controller oligonucleotide and the sequence of the second region of the first oligonucleotide primer can be between 20% and 100%, particularly between 50% and 100%, particularly between 80% and 100%. The binding of the first region of a controller oligonucleotide should particularly take place specifically to the second region of the first oligonucleotide primer under reaction conditions.

Particularly, the sequence of the first region of the controller oligonucleotide is selected such that the number of complementary bases which can bind complementarily with the second region of the first oligonucleotide primers is between 1 and 40, particularly between 3 and 20, particularly between 6 and 15.

Since the controller oligonucleotide is not a template for the polymerase, it can include nucleotide modifications that do not support the polymerase function, which may be base modifications and/or sugar-phosphate backbone modifications. For example, the controller oligonucleotide can include nucleotides and/or nucleotide modifications selected from the following list in its first region: DNA, RNA, LNA (“locked nucleic acids” analogues with 2′-4′ bridge binding in the sugar residue), UNA (“unlocked nucleid acids” without binding between 2′-3′ atoms of the sugar residue), PNA (“peptide nucleic acids” analogues), PTO (phosphorothioates), morpholino analogues, 2′-O-alkyl RNA modifications (such as 2′-OMe, 2′-O-propargyl, 2′-O-(2-methoxyethyl), 2′-O-propyl-amine), 2′-halogen RNA, 2′-amino RNA etc. Said nucleotides or nucleotide modifications are linked to each other, for example by conventional 5′-3′ binding or 5′-2′ binding. For example, a phospho-diester bond or a nuclease-resistant phospho-thioester bond can be used.

The controller oligonucleotide can include nucleotides and/or nucleotide modifications in its first region, wherein the nucleobases are selected from the following list: Adenines and its analogues, Guanines and its analogues, Cytosines and its analogues, Uracil and its analogues, Thymines and its analogues, Inosines or other universal bases (e.g. nitroindole), 2-amino adenine and its analogues, Iso-cytosines and its analogues, Iso-guanines and its analogues.

The controller oligonucleotide can include in its first region non-nucleotide compounds selected from the following list: Intercalating substances that can influence the binding strength between the controller oligonucleotide and the first oligonucleotide primers, e.g. MGB, naphthalenes, etc. The same elements can also be used in the second region of the first primer.

The controller oligonucleotide can include non-nucleotide compounds in its first region, e.g. linkers such as C3, C6, HEG linkers, which link individual segments of the first region.

By means of its second region, the controller oligonucleotide can bind to the first region of the first oligonucleotide primer, wherein the binding is mediated essentially by hybridization of complementary bases.

The length of the second region of the controller oligonucleotide is adapted to the length of the first region of the first oligonucleotide primers and particularly matches it. The length is between about 3-30 nucleotides, particularly between 5 and 20 nucleotides. The sequence of the second region of the controller oligonucleotide is particularly complementary to the first region of the first oligonucleotide primer. The degree of matching in complementarity is between 80% and 100%, especially between 95% and 100%, particularly at 100%. The second region of the controller oligonucleotide includes particularly nucleotide modifications which prevent the polymerase from extending the first oligonucleotide primer but which do not block or do not essentially prevent the formation of complementary double strands, for example 2′-O-alkyl RNA analogs (e.g. 2′-O-Me, 2′-O-(2-methoxyethyl), 2′-O-propyl, 2′-O-propargyl nucleotide modifications), LNA, PNA or morpholino nucleotide modifications. Individual nucleotide monomers are linked particularly via 5′-3′ linkage, but an alternative 5′-2′ linkage between nucleotide monomers can also be used.

The sequence length and its nature of the first and second regions of the controller oligonucleotide are particularly chosen in such a way that the binding of these regions to the first oligonucleotide primer is reversible under reaction conditions at least in one reaction step of the method. This means that although the controller oligonucleotide and the first oligonucleotide primer can bind specifically to each other, this binding should not lead to the formation of a complex of both elements that is permanently stable under reaction conditions.

Rather, an equilibrium between a bound complex form of controller oligonucleotide and the first oligonucleotide primer and a free form of individual components under reaction conditions should be sought or made possible at least in one reaction step. This ensures that at least a portion of the first oligonucleotide primer is present in free form under reaction conditions and can interact with the template to initiate a primer extension reaction. On the other hand, this ensures that respective sequence regions of the controller oligonucleotide are available for binding of a controller oligonucleotide.

The portion of free, single-stranded and thus reactive components can be influenced by temperature selection during the reaction: by lowering the temperature, the first oligonucleotide primer binds to the controller oligonucleotide, so that both participants bind in a complementary double-stranded complex. This allows the concentration of single-stranded forms of individual components to be lowered. An increase in temperature can lead to dissociation of both components into single-stranded forms. In the region of the melting temperature of the complex (controller oligonucleotide/first segment oligonucleotide primer), about 50% of the components are in single stranded form and about 50% as double stranded complex. By using respective temperatures, the concentration of single-stranded forms in the reaction mixture can be influenced.

In an embodiment of the amplification method, which comprises temperature changes between individual reaction steps, the desired reaction conditions can be achieved during the respective reaction steps. For example, the use of temperature regions approximately equal to the melting temperature of complexes of controller oligonucleotide/first oligonucleotide primer can influence portions of respectively free forms of individual components. The temperature used leads to a destabilization of complexes comprising controller oligonucleotide/first oligonucleotide primer, so that during this reaction step individual complex components become at least temporarily single-stranded and are thus enabled to interact with other reaction partners. For example, a first sequence region of the controller oligonucleotide can be released from the double-stranded complex with a non-extended first primer and can thus interact with the second sequence region of an extended first oligonucleotide primer and thus initiate strand displacement. On the other hand, the release of a first, not extended oligonucleotide primer from a complex comprising controller oligonucleotide/first oligonucleotide primer causes the first region of the primer to become single-stranded and can interact with the template to initiate primer extension by a polymerase.

The temperature used does not have to be exactly the same as the melting temperature of the complex of controller oligonucleotide/first oligonucleotide primer. It is sufficient to use a temperature in a reaction step that is approximately in the same region as the melting temperature. For example, the temperature in one of the reaction steps comprises ranges of Tm+/−10° C., particularly Tm+/−5° C., particularly Tm+/−3° C. of the complex of controller oligonucleotide/first oligonucleotide primer.

Such a temperature can be set, for example, as part of the reaction step, which comprises a sequence-specific strand displacement by the controller oligonucleotide.

For embodiments of the amplification method that do not comprise temperature changes between individual reaction steps and where amplification takes place under isothermal conditions, reaction conditions are maintained throughout the entire duration of the amplification reaction under which a balance between a complex form of controller oligonucleotide and the first oligonucleotide primer and a free form of individual components is possible.

The relationship between a complex form of controller oligonucleotide and the first oligonucleotide primer and free forms of individual components can be influenced by reaction conditions (e.g. temperature and Mg2+ concentration) as well as by structures and concentrations of individual components.

The sequence length and its nature of the first and second regions of the controller oligonucleotide are selected in an embodiment such that under given reaction conditions (e.g. in the reaction step of a strand displacement by the controller oligonucleotide) the ratio between a portion of a free controller oligonucleotide and a portion of a controller oligonucleotide in complex with a first oligonucleotide primer comprises the following ranges: from 1:100 to 100:1, particularly from 1:30 to 30:1, specifically from 1:10 to 10:1. The ratio between a portion of a free first oligonucleotide primer and a portion of a first oligonucleotide primer complexed with a controller oligonucleotide comprises ranges from 1:100 to 100:1, particularly from 1:30 to 30:1, particularly r from 1:10 to 10:1.

In an embodiment, the concentration of the first oligonucleotide primer is higher than the concentration of the controller oligonucleotide. Thus, an excess of the first primer is present in the reaction and the controller oligonucleotide must be released from binding of a controller oligonucleotide by selecting a respective reaction temperature. This generally takes place by raising the temperature until sufficient concentrations of free forms of the controller oligonucleotide are present.

In a further embodiment, the concentration of the first oligonucleotide primer is lower than the concentration of the controller oligonucleotide. Therefore, an excess of the controller oligonucleotide is present and the first oligonucleotide primer must be released from binding of a controller oligonucleotide by selecting a respective reaction temperature. This generally takes place by raising the temperature until sufficient concentrations of free forms of the first oligonucleotide primer are present.

Under isothermal conditions, an equilibrium is present: certain portions of the first oligonucleotide primer and controller oligonucleotide are bound to each other, while others are present in the reaction as single-stranded forms.

The controller oligonucleotide can bind to at least one segment of the specifically synthesized extension product of the first oligonucleotide primer by means of its third region. The binding takes place particularly by hybridization of complementary bases between the controller oligonucleotide and the extension product synthesized by the polymerase.

To support the strand displacement reaction, the sequence of the third region should particularly comprise a high complementarity to the extension product. In an embodiment, the sequence of the third region is 100% complementary to the extension product.

The binding of the third region takes place particularly at the segment of the extension product which is immediately adjacent to the first region of the first oligonucleotide primer. Thus, the segment of the extension product is particularly located in the 5′-segment of the total extension product of the first oligonucleotide primer.

Particularly, the binding of the third region of a controller oligonucleotide does not take place over the entire length of the extension product of the first oligonucleotide primer. Particularly one segment at the 3′ end of the extension product remains unbound. This 3′-end segment is necessary for binding the second oligonucleotide primer.

The length of the third region is adjusted in such a way that the third region binds to the 5′ end segment of the extension product and does not bind to the 3′ end segment of the extension product.

The total length of the third region of the controller oligonucleotide is from 2 to 100, particularly from 6 to 60, particularly from 10 to 40 nucleotides or their equivalents. The controller oligonucleotide can undergo a complementary binding with the segment of the extension product over this length and thus displace this 5′ long segment of the extension product from the binding with its complementary template strand.

For example, the length of the 3′-containing segment of the extension product, which is not bound by the controller oligonucleotide, comprises ranges between 20 and 200 nucleotides, particularly between 20 and 100 nucleotides, particularly between 30 and 80, particularly between 30 and 60 nucleotides.

This 3′-standing segment of the extension product is not displaced by the controller oligonucleotide from binding of a controller oligonucleotide with the template strand. Even when the third region of the controller oligonucleotide is fully bound to its complementary segment of the extension product, the first region of the oligonucleotide primer extension product can remain bound to the template strand via its 3′-positioned segment.

In an embodiment, the sequence length of this 3′ segment is such that the two primer extension products do not dissociate when bound by only one controller. Thus, the simultaneous binding of both controllers to their respective primer extension products is required for them to dissociate from each other.

Overall, the controller oligonucleotide has a suitable structure to perform its function: under respective reaction conditions, it is able to displace the extended first oligonucleotide primer from binding of a controller oligonucleotide with the template strand in a sequence-specific manner, thus converting the template strand into a single-stranded form and making it available for further binding with a new first oligonucleotide primer and its target sequence-specific extension by the polymerase.

In order to fulfill the function of strand displacement, regions one, two and three of the controller oligonucleotide should be predominantly in single-stranded form under reaction conditions.

Therefore, double-stranded self-complementary structures (e.g. hairpins) in these regions should be avoided if possible, as they can reduce the functionality of the controller oligonucleotide.

The controller oligonucleotide should not act as a template in the method according to the invention, therefore the first oligonucleotide primer should not be extended by the polymerase when annealed to the controller oligonucleotide under reaction conditions.

This is particularly achieved by using nucleotide modifications which prevent the polymerase from copying the strand. Particularly the 3′-end of the first oligonucleotide primer does not remain extended when the first oligonucleotide primer binds to the controller oligonucleotide under reaction conditions.

The degree of blockage/impediment/slowing/ramping down of the reaction can be between complete expression of this property (e.g. 100% blockage under given reaction conditions) and partial expression of this property (e.g. 30-90% blockage under given reaction conditions). Particularly, nucleotide modifications which are coupled together individually or in a series (e.g. as a sequence fragment consisting of modified nucleotides) more than 70%, particularly more than 90%, particularly more than 95%, and particularly 100% can prevent the extension of a first primer.

The nucleotide modifications may comprise base modifications and/or sugar-phosphate residue modifications. The sugar-phosphate modifications are advantageous because any complementary sequence of a controller oligonucleotide can be assembled by combining it with conventional nucleobases. For example, nucleotides with modifications in the sugar-phosphate moiety that can inhibit or block the synthesis of the polymerase include: 2′-O-alkyl modifications (e.g. 2′-O-methyl, 2′-O-(2-methoxyethyl), 2′-O-propyl, 2′-O-propargyl nucleotide modifications), 2′-O-amino-2′-deoxy nucleotide modifications, 2′-O-aminoalkyl-2′-deoxy nucleotide modifications, PNA, morpholino modifications, etc.

The blockade can take place either by a single nucleotide modification or by coupling several nucleotide modifications in series (e.g. as a sequence fragment consisting of modified nucleotides). For example, at least 2, particularly at least 5, particularly at least 10 of such nucleotide modifications can be coupled side by side in the controller oligonucleotide.

A controller oligonucleotide can comprise a uniform type of nucleotide modification or can comprise at least two different types of nucleotide modification.

The position of such nucleotide modifications in the controller oligonucleotide is particularly intended to prevent the polymerase from extending the 3′ end of a first oligonucleotide primer bound to the controller oligonucleotide.

In an embodiment, such nucleotide modifications are located in the second region of the controller oligonucleotide. In a further embodiment, such nucleotide modifications are located in the third region of the controller oligonucleotide. In an embodiment, such nucleotide modifications are located in the second and third region of the controller oligonucleotide.

For example, the second region of the controller oligonucleotide consists of such nucleotide modifications to at least 20% of its positions, particularly to at least 50%.

For example, the third region of the controller oligonucleotide consists of such nucleotide modifications to at least 20% of its positions, particularly to at least 50%, particularly to at least 90%. In an embodiment, the entire third region comprises nucleotide modifications that prevent a polymerase from extending a primer bound to such a region using the controller oligonucleotide as template. In a further embodiment, the entire third and second region comprise such nucleotide modifications. In a further embodiment, the entire first, second and third region comprise such nucleotide modifications. Thus, the controller oligonucleotide can consist entirely of such nucleotide modifications. Such modified controller oligonucleotides can be used, for example, in multiplex analyses where additional primers are used. This is to prevent unintentional primer extension reactions at one or more controller oligonucleotides.

The sequence of the nucleobases of said nucleotide modifications is adapted to the sequence requirements in the respective region.

The remaining portion can be natural nucleotides or nucleotide modifications that do not or only insignificantly hinder the polymerase function, e.g. DNA nucleotides, PTO nucleotides, LNA nucleotides, RNA nucleotides. Further modifications, e.g. base modifications such as 2-amino adenosine, 2-aminopurine, 5-methyl cytosine, inosine, 5-nitroindole, 7-deaza-adenosine, 7-deaza-guanosine, 5-propyl cytosine, 5-propyl uridine or non-nucleotide modifications such as dyes or MGB modifications etc. can be used, e.g. to adjust the binding strengths of individual regions of the controller oligonucleotides. The individual nucleotide monomers can be coupled to each other via conventional 5′-3′ binding, or alternatively via 5′-2′ binding.

A segment of the controller oligonucleotide with nucleotide modifications that prevent the polymerase from extending from the 3′ end of a first oligonucleotide primer bound to the controller oligonucleotide is called the “second blocking unit”. The length of said segment can include from 1 to 50 nucleotide modifications, particularly from 4 to 30. For example, said segment can be located in the controller oligonucleotide such that the 3′ end of the bound first oligonucleotide primer is located in said segment. Thus, said segment can span the regions two and three.

In an embodiment, particularly no linker structures or spacer structures such as C3, C6, HEG linkers are used to prevent the extension of the 3′ end of a first oligonucleotide primer bound to the controller oligonucleotide.

In an embodiment, a controller oligonucleotide comprises in its third region at least one component of the detection system (e.g. fluorescence reporter or sequencer or a donor fluorophore). The position of this component is located in an embodiment at the 5′ end of the controller oligonucleotide. In another embodiment, said component is located in the inner sequence segment of the third region. The distance to the 5′ end of the controller oligonucleotide can be between 2 and 50 nucleotides, particularly between 2 and 20, particularly between 2 and 10 nucleotides. If the oligonucleotide probe and the controller oligonucleotide are bound by the same first primer extension product at the same time, the two components of the detection system will be in close proximity (e.g. between a fluorescence reporter on the oligonucleotide probe and a donor fluorophore on the controller oligonucleotide).

The controller oligonucleotide can comprise regions one, two and three as well as other sequence segments, which for example flank the above-mentioned regions in the 5′ segment or 3′ segment of the controller oligonucleotide. Such sequence elements can be used for other functions, such as interaction with probes, binding to solid phase, etc. Such regions particularly do not interfere with the function of regions one to three. The length of these flanking sequences can, for example, be between 1 and 50 nucleotides. Furthermore, a controller oligonucleotide can comprise at least one element for immobilization on a solid phase, e.g. a biotin residue. Furthermore, a controller oligonucleotide can comprise at least one element for detection, e.g. a fluorescent dye.

In the presence of a controller oligonucleotide, newly synthesized sequences are verified for their sequence contents by interaction with the controller oligonucleotide.
If there is correct matching with given sequence contents of the controller oligonucleotide, strand displacement takes place, wherein the template strands are replaced by newly synthesized strands. During said process, primer binding sites are converted to the single-stranded state and are available for a new interaction with primers and thus for further synthesis. Thus, the system is converted from both primer extension products into an active state. The controller oligonucleotide thus has an activating effect on the system.
In the absence of matching with given sequence contents of the controller oligonucleotide, the displacement of the template strands is influenced by newly synthesized strands. The strand displacement and/or separation is either quantitatively slowed down or completely eliminated. Thus, primer binding sites are not or less frequently transferred into the single-stranded state. Thus, no or quantitatively less primer binding sites are available for a new interaction with primers. Thus, the system is less often put into an active state from both primer extension products or an active state is not reached at all.

The efficiency of double-strand opening of the newly synthesized primer extension products after each individual synthesis step affects the potentially achievable yields in subsequent cycles: the fewer free/single-stranded primer binding sites of a nucleic acid chain to be amplified are made available at the beginning of a synthesis step, the fewer newly synthesized strands of the nucleic acid chain to be amplified are available in this step. In other words, the yield of a synthesis cycle is proportional to the quantity of primer binding sites available for interaction with respective complementary primers. Generally, a control cycle can be realized.

This control loop generally corresponds to a real-time/on-line control of synthesized fragments: Sequence control takes place in the reaction batch while amplification takes place. Said sequence control takes place according to a predefined pattern and the oligonucleotide system (by strand-opening action of the controller oligonucleotide) can decide between “correct” and “incorrect” states without external interference. In the correct state, the synthesis of sequences is continued, in the non-correct state, the synthesis is either slowed down or completely prevented. The resulting differences in yields of “correct” and “incorrect” sequences after each step affect the overall amplification, which comprises a large number of such steps.

In exponential amplification, this dependence is exponential, so that even small differences in efficiency within a single synthesis cycle due to sequence differences can significantly delay the overall amplification or cause the complete absence of detectable amplification in a given time frame.

This effect of real-time control of the newly synthesized nucleic acid chains is associated with the use of the controller oligonucleotide and the influence of the controller oligonucleotide in the context of an amplification thus extends significantly beyond the length of oligonucleotide primers.

### Reaction Conditions in the Strand Displacement Reaction

The displacement of the second primer extension product from binding with the first primer extension product by means of sequence-dependent strand displacement by the controller oligonucleotide forms a partial step in the amplification. The reaction conditions during this step are adjusted accordingly. The reaction temperature and the reaction time are chosen in such a way that the reaction can take place successfully.

In an embodiment, strand displacement by the controller oligonucleotide proceeds until the second primer extension product is separated/dissociated from the binding of the first primer extension product. Such dissociation of the 3′ segment of the first primer extension product from complementary portions of the second primer extension product can take place spontaneously in a temperature-dependent/temperature-related separation of both primer extension products. Such a dissociation has a positive effect on the kinetics of the amplification reaction and can be influenced by the choice of reaction conditions, e.g. by temperature conditions. The temperature conditions are therefore chosen in such way that successful strand displacement by complementary binding of a controller oligonucleotide favors dissociation of the second primer extension product from the 3′ segment of the first primer extension product.

Due to the action of both controllers, the reaction can proceed with respective speed.

In a further embodiment, the length of the controller and the length of the primer extension products are chosen in such a way that the binding of a single controller oligonucleotide is not sufficient for a separation of both primer extension products. It is particularly advantageous if the newly synthesized strands can only dissociate by simultaneous binding of both controller oligonucleotides to their complementary segments.

In a further embodiment, strand displacement by the controller oligonucleotide proceeds until a 3′ segment of the second oligonucleotide primer extension product (P2.1-Ext) is detached/dissociated from the complementary binding with the first primer extension product (P1. 1-Ext), wherein said segment of the second primer extension product (P2.1-Ext) comprises at least one region complementary to the first primer and one segment complementary to the first primer extension product (P1.1-Ext), which is formed during the enzymatic synthesis. This leads to the formation of a complex (C1.1/P1.1-Ext/P2.1-Ext), which comprises the first primer extension product (P1.1-Ext), the second primer extension product (P2.1-Ext) as well as the controller oligonucleotide (C1.1). In such a complex, the 3′-segment of the second primer extension product is present at least temporarily in single-stranded form, since it can be displaced by the binding of a controller oligonucleotide to the first primer extension product. The 3′ segment of the first primer extension product (P1.1-Ext) is present in such a complex and hybridized to the second primer extension product (P2.1-Ext).

Due to a partially open primer binding site for the first oligonucleotide primer (3′-segment of the second primer extension product), a new primer extension product (P1.2) can bind to a single-stranded sequence segment of the still complexed (P2.1-Ext) under reaction conditions and thus initiate a synthesis of a new first primer extension product (P1.2-Ext) by a polymerase. Generally, said reaction proceeds at reduced speed, since the 3′-segment of the P2.1-Ext is not permanently single-stranded, but is in competitive behavior with the controller oligonucleotide and thus comprises alternating single-stranded and double-stranded states by binding of a controller oligonucleotide to the P1.1-Ext.

Continuation of this newly started synthesis of P1.2-Ext using the second primer extension product as template (P2.1-Ext) can also lead to dissociation of the complex (C1.1/P1.1-Ext/P2.1.-Ext) due to polymerase-related strand displacement. The controller oligonucleotide, temperature-dependent double-strand destabilization and strand displacement by the polymerase act synergistically and complementarily. The result is a dissociation of the 3′ segment of the first primer extension product (P1.1-Ext) from complementary portions of the second primer extension product (P2.1-Ext).

Such a dissociation has a favorable effect on the kinetics of the amplification reaction and can be influenced by the choice of reaction conditions, e.g. by temperature conditions. The participation of polymerase-mediated synthesis-dependent strand displacement in the dissociation of P1.1-Ext and P2.1-Ext has a favorable effect on strand separation.

For example, the temperature in this step comprises ranges from 15° C. to 75° C., particularly from 30° C. to 70° C., particularly from 50° C. to 70° C.

For a given length of the first region of the controller oligonucleotide and the second region of the first oligonucleotide primer (comprising for example regions of 3 to 25 nucleotide monomers, especially 5 to 15 nucleotide monomers), a strand displacement reaction can generally be initiated successfully. If the controller oligonucleotide is fully complementary to respective portions of the first primer extension product, the controller oligonucleotide can bind to the first primer extension product up to the 3′ segment of the first primer extension product and displace the second primer extension product. The second primer extension product thus remains in conjunction with the 3′ segment of the first primer extension product. The strength of this bond can be influenced by temperature. When a critical temperature is reached, said compound can dissociate and both primer extension products can dissociate. The shorter the sequence of the 3′-segment, the more unstable the compound is and the lower the temperature can be, which causes spontaneous dissociation.

A spontaneous dissociation can be achieved, for example, in regions where the temperature is located at about the melting temperature. In an embodiment, the temperature of the strand displacement steps by the controller oligonucleotide is approximately at the melting temperature (Tm+/−3° C.) of the complex comprising the 3′ segment of the first oligonucleotide primer extension product, which is not bound by the controller oligonucleotide, and the second oligonucleotide primer or the second primer extension product.

In an embodiment, the temperature of the strand displacement steps by the controller oligonucleotide is at about the melting temperature (Tm+/−5° C.) of the complex comprising the 3′ segment of the first oligonucleotide primer extension product not bound by the controller oligonucleotide and the second oligonucleotide primer or second primer extension product.

In an embodiment, the temperature of the strand displacement steps by the controller oligonucleotide is above the melting temperature of the complex comprising the 3′-segment of the first oligonucleotide primer extension product not bound by the controller oligonucleotide and the second oligonucleotide primer or second primer extension product. Such a temperature comprises temperature regions from about Tm+5° C. to Tm+20° C., better from Tm+5° C. to Tm+10° C. By using a higher temperature, the equilibrium in this reaction step can be shifted towards dissociation. Thus, the kinetics of the reaction can be favorably influenced. The use of too low temperatures in the strand displacement step using a controller oligonucleotide can lead to a significant slowdown of the amplification.

In an embodiment, a first primer extension product comprises a 3′ segment which is not bound by the controller oligonucleotide and which comprises sequence lengths from 9 to about 18 nucleotides. In said embodiment, spontaneous dissociation can generally be achieved at ranges between 40° C. and 65° C. Higher temperatures also lead to dissociation.

In an embodiment, a first primer extension product comprises a 3′-segment which is not bound by the controller oligonucleotide and which comprises sequence lengths of 15 to about 25 nucleotides. In said embodiment, spontaneous dissociation can generally be achieved in ranges between 50° C. and 70° C. Higher temperatures also lead to dissociation.

In an embodiment, a first primer extension product comprises a 3′-segment which is not bound by the controller oligonucleotide and which comprises sequence lengths of 20 to about 40 nucleotides.

In said embodiment, spontaneous dissociation can generally be achieved in ranges between 50° C. and 75° C. Higher temperatures also lead to dissociation.

The composition of the 3′-segment of the first primer extension product and, if necessary, the introduction of oligonucleotide modifications (e.g. MGB) or reaction conditions (e.g. TPAC, betaines) that influence the melting temperature can influence the choice of temperature. A respective adjustment can therefore be made.

In an embodiment, all steps of the amplification process take place under stringent conditions that prevent or slow down the formation of unspecific products/by-products. Such conditions include higher temperatures, for example above 50° C.

In an embodiment, the individual steps of strand displacement by controller oligonucleotides take place at the same temperature as the synthesis of the first and second primer extension product. In a further embodiment, the individual strand displacement steps are carried out by controller oligonucleotides at a temperature that differs from the temperature of the respective synthesis of the first and second primer extension products. In a further embodiment, the synthesis of the first primer extension product and the strand displacement by the controller oligonucleotide takes place at the same temperature. In a further embodiment, the synthesis of the second primer extension product and the displacement of the strand by the oligonucleotide controller is carried out at the same temperature.

The concentration of the controller oligonucleotide comprises regions from 0.01 μmol/l to 50 μmol/l, particularly from 0.1 μmol/l to 20 μmol/l, particularly from 0.1 μmol/l to 10 μmol/l.

### EXAMPLES

### Example 1: Creation of a Start Nucleic Acid Chain Using a Primer Extension Starting from Double-Stranded Nucleic Acid Chains

This example shows the production of a start nucleic acid chain (M1.1 and M 2.1) with a segment M1.1.6 or M2.1.6 comprising modified nucleotides (FIG. 9 A-D and FIG. 20 A-D) The production of the start nucleic acid took place by primer extension starting from double-stranded nucleic acid chains comprising the target sequences. Primers were used, which were also used as first and second primer in the later amplification.

Double-stranded nucleic acid chains (comprising target sequences and corresponding complementary strands) were added to the reaction. Both strands can be used as templates for primer extension. The double-stranded templates were converted to single-stranded form by initial denaturation under reaction conditions. In the subsequent reaction step, primers (which were also used in the subsequent amplification) were hybridized to their complementary sequence segments and extended by Taq polymerase. These steps were repeated once (denaturation and primer extension).

After the reaction was completed, the resulting reaction mixtures comprised both primer extension products (the first and second primer extension products), which were used as start nucleic acid chains in the amplification method (example 2). By using both primers (which are also used in amplification, example 2) the resulting products also comprised the first and second regions of the respective primer.

The primer extension reaction based on double-stranded nucleic acid chains was performed as follows:

The first and second primers were used in concentrations of 1 μmol/l respectively. DNA templates were used in concentrations of approximately 0.05 to 0.5 nmol/l.

The further reaction conditions were:

1× isothermal buffer (New England Biolabs, catalog #B0537S; in single concentration the buffer contains: 20 mM Tris-HC; 10 mM (NH4)2SO4; 50 mM KCl; 2 mM MgSO4; 0.1% Tween® 20; pH 8.8@25° C.); dNTP (dATP, dCTP, dUTP, dGTP), each 200 μmol/l;

The production of the start nucleic acid chains took place with Taq polymerase (NEB). The Taq polymerase was present in the reaction mixture in single concentration (approx. 100-fold dilution of the stock solution).

The thermal reaction conditions were as follows:
Initial denaturation at 95° C. for 3 minutes.
Annealing and elongation at 50° C. for 1 h.
Denaturation at 95° C. for 1 minute.
Annealing and elongation at 50° C. for 1 h.

### Primer:

The first oligonucleotide primer:
P1F5-50-AE2051
(SEQ ID NO 001)
5′ CACAATCATCAATACTTTTTTTACCTGTA 7878 658678871TACCTGTATTCC 3′

The underlined sequence part (position 1-12 from the 3′ end) corresponds to the respective first region of a primer and can hybridize sequence-specifically to a template and be extended by the polymerase.

The numbered positions stand for modified nucleotide analogues as building blocks of the chain:

5=2′-O-Me A (2′-O-methyl adenosine)
6=2′-O-Me G (2′-O-methyl guanosine)
7=2′-O-Me C (2′-O-methyl cytosine)
8=2′-O-Me U (2′-O-methyl uridine)

This oligonucleotide primer comprises the first region (positions 1-12 from the 3′-end), the second region (C3 linker, as well as positions 13-24 from the 3′-end), as well as a segment with an additional sequence variant P1 (positions 25-57 from the 3′-end). The first region and the second region are necessary for performing a specific amplification and can be summarized as “basic structure of the first primer” or “minimal structure of the first primer”. The additional sequence variant P1 is an example of additional segments which can be integrated at the first oligonucleotide primer. Positions 1-12 serve as templates for the synthesis of the second primer extension product. C3 modification and the second region prevent continuation of the synthesis at positions 25-57 during synthesis of the second primer extension product.

The second oligonucleotide primers:
P1F5G2-1001-103_2xInv
(SEQ ID NO 002)
5′ CACAATCATCAATACTTTTTTGTCAACCAT87876856 87871GTCAACCATAAT 3′

The underlined sequence part (position 1-12 from the 3′ end) corresponds to the respective first region of a primer and can hybridize sequence-specifically to a template and be extended by the polymerase.

Both primers comprised the following composition:

A=2′-deoxy adenosine; C=2′-deoxy cytosine; G=2′-deoxy guanosine; T=2′-deoxy thymidine

Modifications (this sequence segment corresponds to the respective second region of a primer):

### 1=C3-Linker

The numbered positions stand for modified nucleotide analogues as building blocks of the chain:

5=2′-O-Me A (2′-O-methyl adenosine)
6=2′-O-Me G (2′-O-methyl guanosine)
7=2′-O-Me C (2′-O-methyl cytosine)
8=2′-O-Me U (2′-O-methyl uridine)

The nucleotides and modifications are linked together with phosphodiester bonds.

Double-stranded templates were used, which comprised subsequent target sequences. The double-stranded templates were prepared by conventional PCR. The Tm of double strands was approx. 79 to 81° C. (measured at the end of the PCR reaction).

Due to the complementarity of the two strands, only one strand of each of the double-stranded templates is given below (one strand comprises one target sequence):

M2HAF5-2xC-GC-Gap = 10
(SEQ ID NO 003)
5′ AAGTCAACCA TAATGGACTA CTTCGAGCAG ATCCATTAGA GGTCTGGACA
GGCAAGGAAT ACAGGTATTT TGAA 3′
M2HAF5-2xC-GC-Gap = 20
(SEQ ID NO 004)
5′ AAGTCAACCA TAATGGACTA CTTCGAGCAG ATCCATTAGC TCCTACGCCA
GGTCTGGACA GGCAAGGAAT ACAGGTATTT TGAA 3′
M2HAF5-2xC-GC-Gap = 30
(SEQ ID NO 005)
5′ AAGTCAACCA TAATGGACTA CTTCGAGCAG ATCCATTAG CTCCTCTGGA
TATAGACGCCA GGTCTGGACA GGCAAGGAAT ACAGGTATTT TGAA 3′
M2HAF5-2xC-Ex-Gap = 30
(SEQ ID NO 006)
5′ AAGT CAAC CATA ATAT CATG AGAG ACAT CGCC TCTGG GCTA ATAA TTAG CTCC
TCTG GATA TAGA CGCC AGGT GGAC TACTT CTAA TCTG TAAG AGCA GATC CCTG GACA
GGCA AGGA ATAC AGGT ATTT TGAA 3′
M2HAF5-2xC-Ex-Gap = 20
(SEQ ID NO 007)
5′ AAGTC AACCA TAATA TCATG AGAGA CATCG CCTCT GGGCT AATAA TTAGC TCCTA
CGCCA GGTGG ACTAC TTCTA ATCTG TAAGA GCAGA TCCCT GGACAG GCAAG GAATAC
AGGTA TTTTG AA 3′
M2HAF5-2xC-Ex-Gap = 0
(SEQ ID NO 008)
5′ AAGTC AACCA TAATA TCATGA GAGAC ATCGC CTCTG GGCTA ATAGG ACTAC TTCTA
ATCTG TAAGA GCAGA TCCCT GGACA GGCAA GGAAT ACAGG TATTT TGAA 3′

As a result of primer extension, the following products were obtained (start nucleic acid chains M1.1 and M2.1):

mixture 1 (using template M2HAF5-2xC-GC-Gap = 10)
Product: M2HAF5-2xC-GC-Gap = 10_THL (Start nucleic acid chain M2.1)
(SEQ ID NO 009)
5′ CACAA TCATCA ATACT TTTTT GTCAA CCAT8 78768 56878 71GTC AACCA TAATG
GACTA CTTCG AGCAG ATCAT TAGAG GTCTG GACAGG CAAGG AATAC AGGTA TTTTG
AA 3′
Product: M2HAF5-2xC-GC-Gap = 10_THR (Start nucleic acid chain M1.1)
(SEQ ID NO 010)
5′ CACAATCATCAATACTTTTTTTACCTGTAT7878
658678871TACCTGTATTCCTTGCCTGTCCAGACCTCTAATGGATCTGCTCGAAGTAGTCCAT
TATGGTTGACTT 3′
Mixture 2 (using template M2HAF5-2xC-GC-Gap = 20)
Product: M2HAF5-2xC-GC-Gap = 20_THL (Start nucleic acid chain M2.1)
(SEQ ID NO 011)
5′ CACAATCATCAATACTTTTTTGTCAACCAT87876856
87871GTCAACCATAATGGACTACTTCGAGCAGATCCATTAGCTCCTACGCCAGGTCTGGACAGGC
AAGGAATACAGGTATTTTGAA 3
Product: M2HAF5-2xC-GC-Gap = 20_THR (Start nucleic acid chain M1.1)
(SEQ ID NO 012)
5′ CACAATCATCAATACTTTTTTTACCTGTAT7878
658678871TACCTGTATTCCTTGCCTGTCCAGACCTGGCGTAGGAGCTAATGGATCTGCTCG
AAGTAGTCCATTATGGTTGACTT 3′
Mixture 3 (using template M2HAF5-2xC-GC-Gap = 30)
Product: M2HAF5-2xC-GC-Gap = 30_THL (Start nucleic acid chain M2.1)
(SEQ ID NO 013)
5′ CACAATCATCAATACTTTTTTGTCAACCAT87876856
87871GTCAACCATAATGGACTACTTCGAGCAGATCCATTAGCTCCTCTGGATATAGACGCCA
GGTCTGGACAGGCAAGGAATACAGGTATTTTGAA 3′
Product: M2HAF5-2xC-GC-Gap = 30_THR (Start nucleic acid chain M1.1)
(SEQ ID NO 014)
5′ CACAATCATCAATACTTTTTTTACCTGTAT7878
658678871TACCTGTATTCCTTGCCTGTCCAGACCTGGCGTCTATATCCAGAGGAGCTAATGG
ATCTGCTCGAAGTAGTCCATTATGGTTGACTT 3′
Mixture 4 (using template M2HAF5-2xC-Ex-Gap = 30)
Product: M2HAF5-2xC-Ex-Gap = 30_THL (Start nucleic acid chain M2.1)
(SEQ ID NO 015)
5′ CACAATCATCAATACTTTTTTGTCAACCAT87876856
87871GTCAACCATAATATCATGAGAGACATCGCCTCTGGGCTAATAATTAGCTCCTCTGGAT
ATAGACGCCAGGTGGACTACTTCTAATCTGTAAGAGCAGATCCCTGGACAGGCAAGGAATA
CAGGTATTTTGAA 3′
Product: M2HAF5-2xC-Ex-Gap = 30_THR (Start nucleic acid chain M1.1)
(SEQ ID NO 016)
5′ CACAATCATCAATACTTTTTTTACCTGTAT7878
658678871TACCTGTATTCCTTGCCTGTCCAGGGATCTGCTCTTACAGATTAGAAGTAGTCCA
CCTGGCGTCTATATCCAGAGGAGCTAATTATTAGCCCAGAGGCGATGTCTCTCATGATATTA
TGGTTGACTT 3′
Mixture 5 (using template M2HAF5-2xC-Ex-Gap = 20)
Product: M2HAF5-2xC-Ex-Gap = 20_THL (Start nucleic acid chain M2.1)
(SEQ ID NO 017)
5′ CACAATCATCAATACTTTTTTGTCAACCAT87876856
87871GTCAACCATAATATCATGAGAGACATCGCCTCTGGGCTAATAATTAGCTCCTACGCCA
GGTGGACTACTTCTAATCTGTAAGAGCAGATCCCTGGACAGGCAAGGAATACAGGTATTTTG
AA 3′
Product (SEQ ID NO 018):
M2HAF5-2xC-Ex-Gap = 20_THR (Start nucleic acid chain M1.1)
5′ CACAATCATCAATACTTTTTTTACCTGTAT7878
658678871TACCTGTATTCCTTGCCTGTCCAGGGATCTGCTCTTACAGATTAGAAGTAGTCCA
CCTGGCGTAGGAGCTAATTATTAGCCCAGAGGCGATGTCTCTCATGATATTATGGTTGACTT
3′
Mixture 6 (using template M2HAF5-2xC-Ex-Gap = 0)
Product:
M2HAF5-2xC-Ex-Gap = 0_THL (Start nucleic acid chain M2.1)
(SEQ ID NO 019)
5′ CACAATCATCAATACTTTTTTGTCAACCAT87876856
87871GTCAACCATAATATCATGAGAGACATCGCCTCTGGGCTAATAGGACTACTTCTAATCT
GTAAGAGCAGATCCCTGGACAGGCAAGGAATACAGGTATTTTGAA 3′
Product:
M2HAF5-2xC-Ex-Gap = 0_THR (Start nucleic acid chain M1.1)
(SEQ ID NO 020)
5′ CACAATCATCAATACTTTTTTTACCTGTAT7878
658678871TACCTGTATTCCTTGCCTGTCCAGGGATCTGCTCTTACAGATTAGAAGTAGTCCT
ATTAGCCCAGAGGCGATGTCTCTCATGATATTATGGTTGACTT 3′
A = 2′-deoxy adenosine; C = 2′-deoxy cytosine; G = 2′-deoxy guanosine;
T = 2′-deoxy thymidine (thymidine)

Modifications: The numbered positions stand for modified nucleotide analogues as building blocks of the chain:

### 1=C3-Linker

5=2′-O-Me A (2′-O-methyl adenosine)
6=2′-O-Me G (2′-O-methyl guanosine)
7=2′-O-Me C (2′-O-methyl cytosine)
8=2′-O-Me U (2′-O-methyl-uridine)

The resulting products of the primer extension reaction correspond to the respective start nucleic acid chains (M1.1 and M2.1) and comprise either the target sequence or its complementary sequence, as well as primer binding sites to which primers can bind during amplification. These products were used in the amplification as a mixture of both primer extension products (example 2).

### Example 2: Exponential Amplification of Amplification Fragments from Start Nucleic Acid Chains

The amplification was performed using two sequence-specific controller oligonucleotides, two sequence-specific primers and respectively two auxiliary oligonucleotides (block oligonucleotides).

### Oligonucleotides Used:

### Primer:

The first oligonucleotide primer:
P1F5-50-AE2051
(SEQ ID NO 001)
5′ CACAATCATCAATACTTTTTTTACCTGTAT 7878 658678871TACCTGTATTCC 3′

The underlined sequence part (position 1-12 from the 3′ end) corresponds to the respective first region of a primer and can be hybridized sequence-specifically to a template and extended by the polymerase.

The second oligonucleotide primer:
P1F5G2-1001-103_2xInv
(SEQ ID NO 002)
5′ CACAATCATCAATACTTTTTTGTCAACCAT87876856 87871GTC
AACCATAAT 3′

The underlined sequence part (position 1-12 from the 3′ end) corresponds to the respective first region of a primer and can hybridize sequence-specifically to a template and be extended by the polymerase.

Both primers comprised the following composition:

A=2′-deoxy adenosine; C=2′-deoxy cytosine; G=2′-deoxy guanosine; T=2′-deoxy thymidine (thymidine)

modifications: The numbered positions stand for modified nucleotide analogues as building blocks of the chain:

1=C3-linker
5=2′-O-Me A (2′-O-methyl adenosines)
6=2′-O-Me G (2′-O-methyl guanosines)
7=2′-O-Me C (2′-O-methyl cytosines)
8=2′-O-Me U (2′-O-methyl-uridines)

The first block oligonucleotide:
BP1F5-25001-402
(SEQ ID NO 021)
5′ 7878 65867887 857786 1 TATTAA X 3′
The second block oligonucleotide:
B1-P1F5G2-3501-304_2xInv
(SEQ ID NO 022)
5′- 87876856 8787 68 755 2 CCATAA TG AAA X 3′
A = 2′-deoxy adenosine; C = 2′-deoxy cytosine;
G = 2′-deoxy guanosine; T = 2′-deoxy thymidine
(thymidine)

modifications: The numbered positions stand for modified nucleotide analogues as building blocks of the chain:

1=C3-linker

### 2=HEG-linker

5=2′-O-Me A (2′-O-methyl adenosine)
6=2′-O-Me G (2′-O-methyl guanosine)
7=2′-O-Me C (2′-O-methyl cytosine)
8=2′-O-Me U (2′-O-methyl-uridine)
X=3′-phosphate group to block a possible extension by polymerase.

The first controller oligonucleotide:
CF5-1001-11-6-S
(SEQ ID NO 023)
5′ 5 7866575667 55 665585756 GTA GAAGCATC AGAG
X 3′
The second controller oligonucleotide:
CF-1001-L-103_2xInv-GC-S
(SEQ ID NO 024)
5′ 5 6658786787 6556856877 5 885866 TTG AC GAGA
CTACGAGA AG X 3′

For the amplification of M2HAF5-2xC-Ex-Gap=30_THL, M2HAF5-2xC-Ex-Gap=30_THR, M2HAF5-2xC-Ex-Gap=20_THL, M2HAF5-2xC-Ex-Gap=20_THR, M2HAF5-2xC-Ex-Gap=0_THL und M2HAF5-2xC-Ex-Gap=0_THR the following two different controller oligonucleotides were used:

The first controller oligonucleotide:
CF5-1001-11-6
(SEQ ID NO 025)
5′ 5 6657857887 8558786855 6567565877 7866575667
55 665585756 GTA GAAGCATC AGAG X 3′
The second controller oligonucleotide:
CF5G2-1001-401_2xInvEx
(SEQ ID NO 026)
5′ 5 858856777565667658687878758658 5 885866
TTG AC GAGA CTACGAGA AG X 3′
A = 2′-deoxy adenosine; C = 2′-deoxy cytosine;
G = 2′-deoxy guanosine; T = 2′-deoxy thymidine

Modifications: The numbered positions stand for modified nucleotide analogues as building blocks of the chain:

5=2′-O-Me A (2′-O-methyl adenosine)
6=2′-O-Me G (2′-O-methyl guanosine)
7=2′-O-Me C (2′-O-methyl cytosine)
8=2′-O-Me U (2′-O-methyl-uridine)
X=3′-phosphate group to block a possible extension by polymerase.

The reaction was performed under the following conditions: The first and second primers were used in concentrations of 0.5 μmol/l respectively. The first and second block oligonucleotide were used in concentrations of 5 μmol/l respectively. The first and second controller oligonucleotide were used in concentrations of 2 μmol/l respectively. Mixtures with DNA templates (start nucleic acid chains M1.1 and M2.1) from example 1 (mixture 1 to 6) were used individually, respectively diluted 6-fold and/or 6000-fold.

The further reaction conditions were:
1× Isothermal Buffer (New England Biolabs, catalog #B0537S; in single concentration the buffer contains: 20 mM Tris-HC; 10 mM (NH4)2SO4; 50 mM KCl; 2 mM MgSO4; 0.1% Tween®20; pH 8.8@25° C.); dNTP (dATP, dCTP, dUTP, dGTP), each 200 μmol/l;
EvaGreen dye (Jena Biosciences) in 1:50 dilution.

The amplification of the BST 2.0 polymerase (stock solution: Bst 2.0 WarmStart 120,000 units/ml, NEB) took place according to the manufacturer's instructions. The BST polymerase was present in the reaction mixture in 1200-fold dilution of the polymerase stock solution.

The thermal reaction conditions were as follows:
Initial phase at 65° C. for 2 minutes.
125 cycles with the following structure
Phase at 50° C. for 2 min.
Phase at 65° C. for 2 min.
Denaturation at 95° C. for 3 minutes.
Recording of a melting curve.

### Results and Evaluation:

Analysis of Amplification Fragments after Exponential Amplification of the Start Nucleic Acid Chains

The following techniques were used to analyze the amplification reaction and to evaluate the resulting amplification products:
Fluorescence signal of an intercalating dye (EvaGreen)
Melt curve analysis of the resulting amplification products.
   Amplification of M2HAF5-2xC-GC-Gap=10_THL and M2HAF5-2xC-GC-Gap=10_THR Products from Start Nucleic Acid Chains

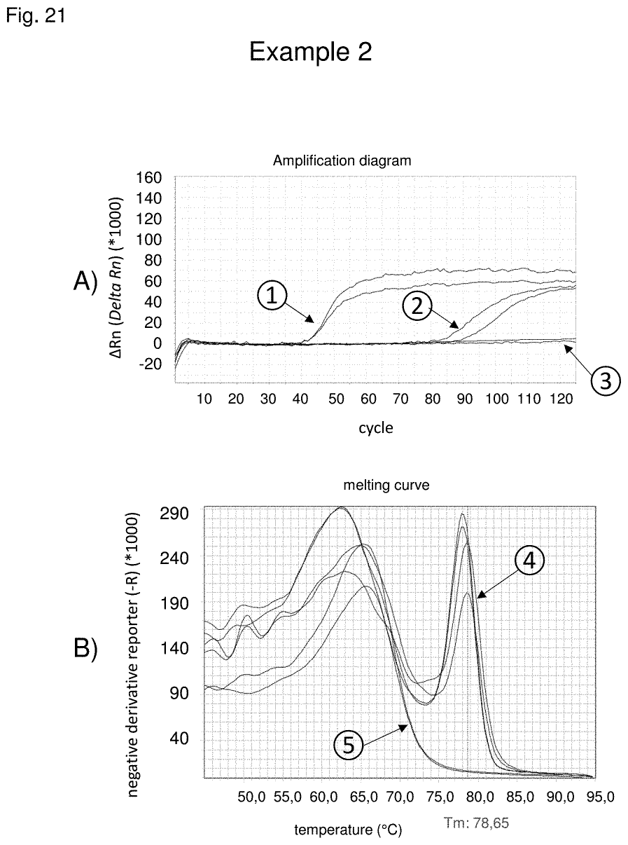

FIG. 21 (A) shows a typical course of the EvaGreen fluorescence signal during the amplification reaction. On the Y-axis the change of the fluorescence signal of the EvaGreen dye and on the X-axis the reaction time (as cycle number) is plotted. The increase of the fluorescence signal indicates that amplification products have been formed. Arrow 1 belongs to the 6-fold and (arrow 2) to the 6000-times diluted starting material from example 1. Arrow 3 shows the course of a negative control, here H₂O was used instead of the starting material from example 1.

FIG. 21 (B) shows the melting curves belonging to the batches. The Y-axis shows the derivation of the fluorescence signal and the X-axis the temperature. A peak is visible, indicating that a uniform amplification product has been formed. The peaks marked with arrow 4 belong to the 6-fold and 6000-fold diluted starting material from example 1. The curves marked with arrow 5 do not comprise a product peak and belong to the negative control.

Amplification of M2HAF5-2xC-GC-Gap=20_THL and M2HAF5-2xC-GC-Gap=20_THR Products from Start Nucleic Acid Chains

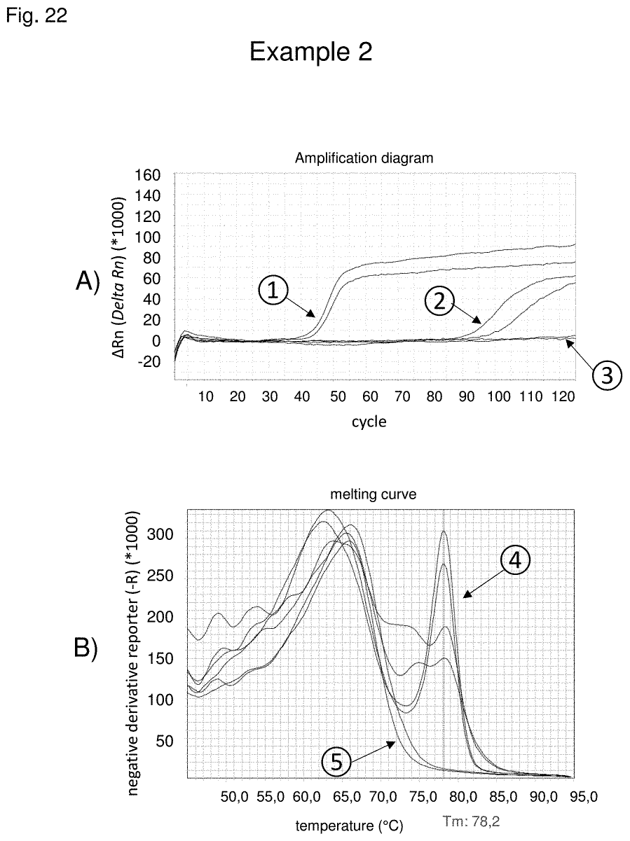

FIG. 22 (A) shows a typical course of the EvaGreen fluorescence signal during the amplification reaction. On the Y-axis the change of the fluorescence signal of the EvaGreen dye and on the X-axis the reaction time (as cycle number) is plotted. The increase of the fluorescence signal indicates that amplification products have been formed. Arrow 1 belongs to the 6-fold and arrow 2 to the 6000-times diluted starting material from example 1. Arrow 3 shows the course of a negative control, here H₂O was used instead of the starting material from example 1.

FIG. 22 (B) shows the melting curves belonging to the batches. The Y-axis shows the derivation of the fluorescence signal and the X-axis the temperature. A peak is visible, indicating that a uniform amplification product has been formed. The peaks marked with arrow 4 belong to the 6-fold and 6000-fold diluted starting material from example 1. The curves marked with arrow 5 do not comprise a product peak and belong to the negative control.

Amplification of M2HAF5-2xC-Ex-Gap=30_THL and M2HAF5-2xC-Ex-Gap=30_THR Products from Start Nucleic Acid Chains

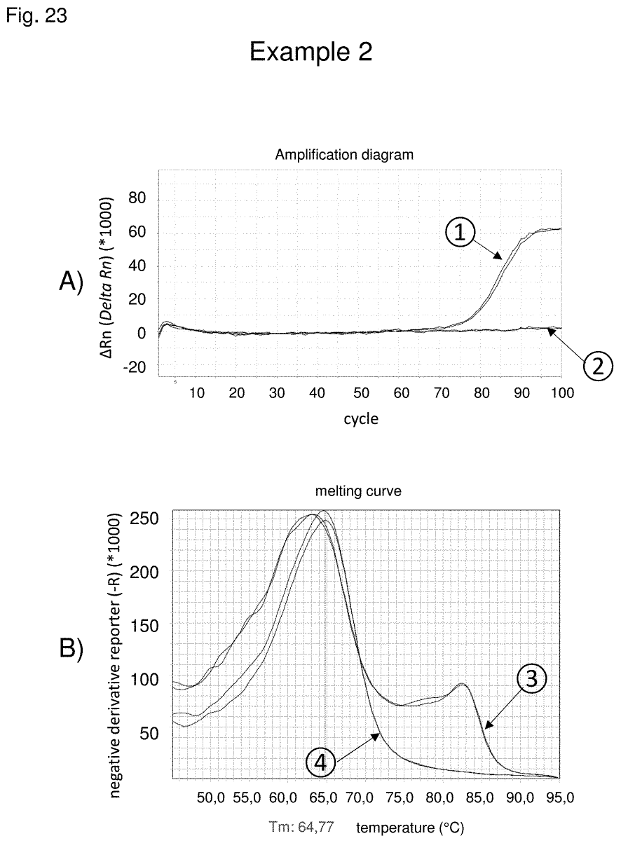

FIG. 23 (A) shows a typical course of the EvaGreen fluorescence signal during the amplification reaction. On the Y-axis the change of the EvaGreen fluorescence signal and on the X-axis the reaction time (as cycle number) is plotted. The increase of the fluorescence signal indicates that amplification products have been formed. Arrow 1 belongs to the 6000-times diluted starting material from example 1. Arrow 2 shows the course of a negative control, here H₂O was used instead of the starting material from example 1.

FIG. 23 (B) shows the melting curves belonging to the batches. The Y-axis shows the derivation of the fluorescence signal and the X-axis the temperature. A peak is visible, indicating that a uniform amplification product has been formed. The peaks marked with arrow 3 belong to the starting material diluted 6000-fold in Example 1. The curves marked with arrow 4 do not comprise a product peak and belong to the negative control.

Amplification of M2HAF5-2xC-Ex-Gap=20_THL and M2HAF5-2xC-Ex-Gap=20_THR Products from Start Nucleic Acid Chains

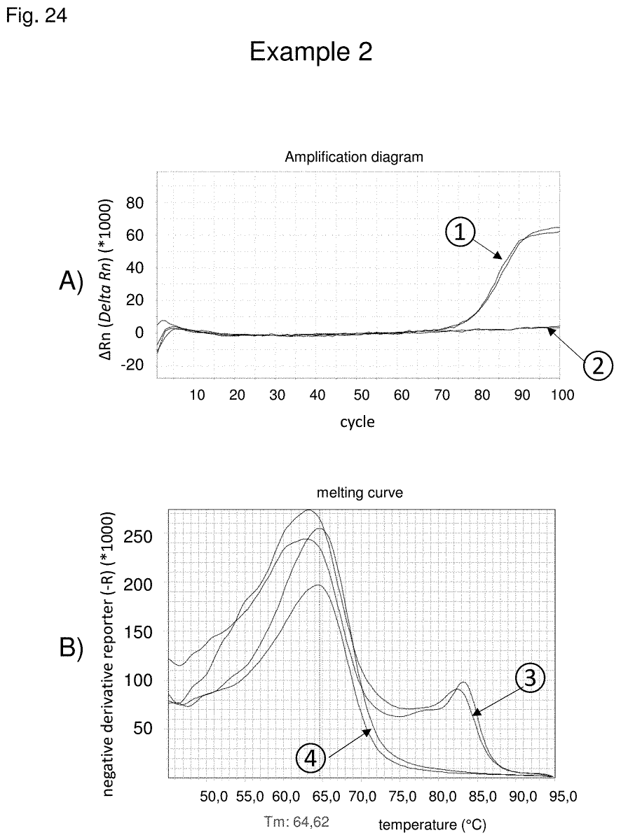

FIG. 24 (A) shows a typical course of the EvaGreen fluorescence signal during the amplification reaction. On the Y-axis the change of the fluorescence signal of the EvaGreen dye and on the X-axis the reaction time (as cycle number) is plotted. The increase of the fluorescence signal indicates that amplification products have been formed. Arrow 1 belongs to the 6000-times diluted starting material from example 1. Arrow 2 shows the course of a negative control, here H₂O was used instead of the starting material from example 1.

FIG. 24 (B) shows the melting curves belonging to the batches. The Y-axis shows the derivation of the fluorescence signal and the X-axis the temperature. A peak is visible, indicating that a uniform amplification product has been formed. The peaks marked with arrow 3 belong to the starting material diluted 6000-fold in Example 1. The curves marked with arrow 4 do not comprise a product peak and belong to the negative control.

Amplification of M2HAF5-2xC-Ex-Gap=0THL and M2HAF5-2xC-Ex-Gap=0THR Products from Start Nucleic Acid Chains

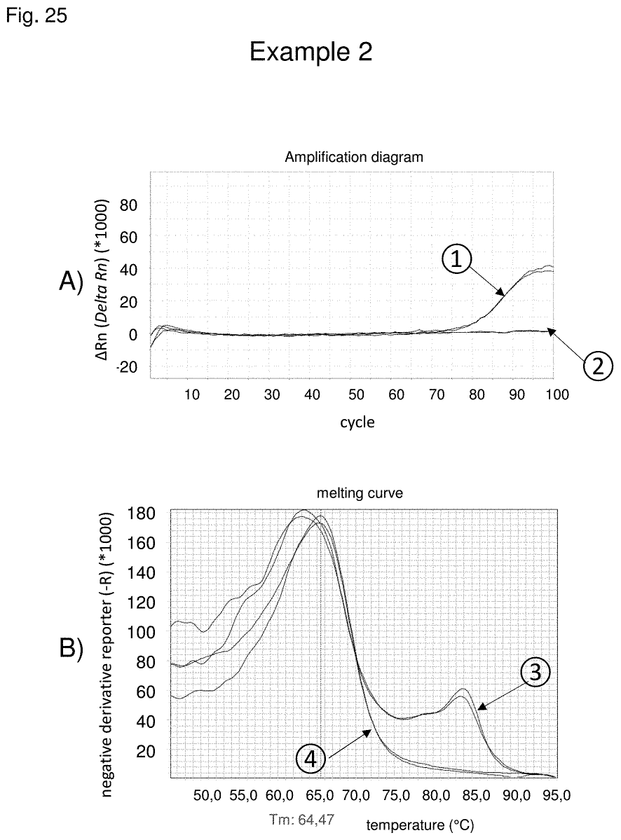

FIG. 25 (A) shows a typical course of the EvaGreen fluorescence signal during the amplification reaction. On the Y-axis the change of the EvaGreen fluorescence signal and on the X-axis the reaction time (as cycle number) is plotted. The increase of the fluorescence signal indicates that amplification products have been formed. Arrow 1 belongs to the 6000-times diluted starting material from example 1. Arrow 2 shows the course of a negative control, here H₂O was used instead of the starting material from example 1.

FIG. 25 (B) shows the melting curves belonging to the batches. The Y-axis shows the derivation of the fluorescence signal and the X-axis the temperature. A peak is visible, indicating that a uniform amplification product has been formed. The peaks marked with arrow 3 belong to the starting material diluted 6000-fold in Example 1. The curves marked with arrow 4 do not comprise a product peak and belong to the negative control.

### Use of Auxiliary Oligonucleotides (Block Oligonucleotides):

In this example, an advantageous embodiment of the reaction conditions was chosen in which, on the one hand, the primer concentration was lower than the concentration of controller oligonucleotide and, on the other hand, cyclic temperature changes (between 50° C. and 65° C.) were used.

In the oligonucleotides used, oligonucleotide primers form an interaction pair with the respective controller oligonucleotides (e.g. first oligonucleotide primer and first controller oligonucleotide). Said interaction pair had a melting temperature of about 63° C. under amplification reaction conditions (measured at about 1 μmol/l concentration of both components under amplification buffer conditions). The first region of the oligonucleotide primer formed a complex with a complementary sequence portion of a template at a melting temperature of about 50° C. (e.g. first region of the first primer and the second primer extension product (P2.1-Ext) measured at about 1 μmol/l concentration of both components under buffering conditions of the amplification).

In a certain embodiment, primer-controller combinations can be used where the concentration of primer is higher than the concentration of controller (e.g. primer 2-5 μmol/l and controller 1 μmol/l). In such an embodiment an excess of primer is present, so that the primer extension step at a temperature of 50° C. is in good yields, despite partial binding of an oligonucleotide primer to the controller oligonucleotide.

In a further embodiment (example 2) a mixture of a primer and a respective controller was used in which the concentration of primer was lower than the concentration of the controller oligonucleotide (e.g. primer 0.5 μmol/l and controller 2 μmol/l). Thus, the controller oligonucleotide was present in excess. In this embodiment, sending the reaction temperature to 50° C. led to the rapid binding of an oligonucleotide primer to the controller oligonucleotides. Said reduced the yield in the primer extension step at 50° C. and lowered the speed of amplification. To maintain a sufficient primer concentration even at low temperatures and thus increase yields in the primer extension step, so-called block oligonucleotides were used. Such block oligonucleotides competed with the oligonucleotide primer for binding of a controller oligonucleotide, but were not able to be extended by a polymerase.

By using block oligonucleotides it was possible to use the combination of oligonucleotide primer and controller oligonucleotide at certain (“at certain”<-is rather imprecise Should we formulate more precisely here, e.g. in specifically low primer concentrations) concentration regions and combine them with cyclic temperature changes. This led to an increase of the reaction speed.

The structure of block oligonucleotides is essentially similar to the structure of oligonucleotide primers, with the following differences:
Block oligonucleotides are not extended by polymerase. This can be achieved, for example, by blocking the 3′-end by modification (e.g. 3-phosphate, 3′-C3, dideoxi nucleotide), and/or by introducing terminal mismatches into 3′-end of the block oligonucleotide.
Block oligonucleotides may differ in sequence composition from their corresponding primers. The number of sequence mismatches can vary from 1 nucleotide to 20 nucleotides.

When designing block oligonucleotides, it is advantageous to keep the Tm of block oligonucleotides to controller oligonucleotides in the same range as the Tm of oligonucleotide primers and controller oligonucleotides. As a result, block oligonucleotides and oligonucleotide primers compete for binding to controller oligonucleotides to a similar extent (e.g. Tm of primer-controller complex plus/minus 3° C.). By using higher concentrations of block oligonucleotides than oligonucleotide primers, the binding ratio can be advantageously influenced.

FIG. 1 shows schematically the sequence components of an embodiment of the invention. The primers 1.1 and primers 2.1 can bind predominantly complementarily with their respective first regions (in 3′-segments) to the respective primer binding sites in amplification products and can be extended by the polymerase, wherein the primer extension products P1.1-Ext and P2.1-Ext are formed. The polynucleotide tail (primer overhang) of the second region of the respective primer does not bind to amplification fragments 1.1 formed during amplification and is not copied by the polymerase. Strand separation after a respective polymerase-dependent synthesis procedure takes place with the participation of controller 1.1 and controller 2.1, which can bind to the respectively complementary segments of P1.1-Ext and P2.1-Ext. Amplification products represent the result of a template-dependent synthesis by the polymerase (primer extension) and may themselves appear as templates in subsequent cycles (primer extension products, also called primer extension products, P1.1-Ext and P2.1-Ext). The amplification product 1.1 comprises P1.1-Ext and P2.1-Ext. C1.1 can bind complementarily to the complementary segment of P1.1-Ext. C2.1 can bind to the complementary segment of the P2.1 Ext. The middle segment 3 is not bound complementarily by any controller.

The first primer extension product (P1.1-Ext) comprises the following segments (in 5′-3′ direction): P1.1E6, P1.1E5, P1.1E4, P1.1E3, P1.1E2, P1.1.E1. Segments P1.1E6 and P1.1E5 are formed by the first oligonucleotide primers, segments P1.1 E4 to P1.1E1 are synthesized by the polymerase during amplification.

The second primer extension product (P2.1-Ext) comprises the following segments (in 5′-3′ direction): P2.1E6, P2.1E5, P2.1E4, P2.1E3, P2.1E2, P2.1 E1. Segments P2.1E6 and P2.1E5 are formed by the second oligonucleotide primer, segments P1.1E4 to P1.1E1 are synthesized by the polymerase during amplification.

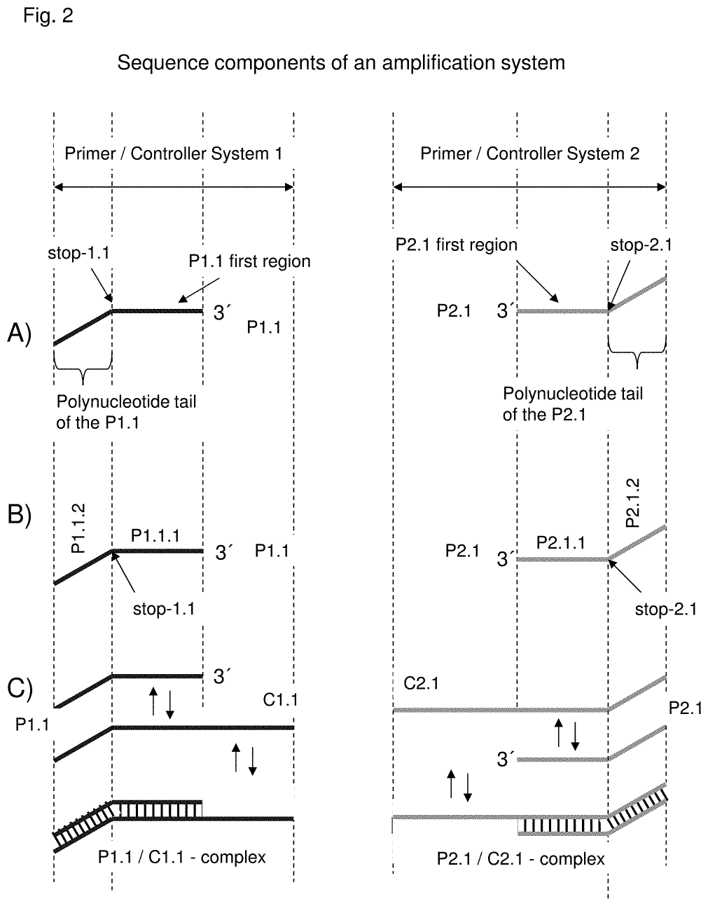

FIG. 2 shows schematically the topography of components in an embodiment of the invention.

The first oligonucleotide primer (P1.1) comprises a first region (P1.1.1) and a second region (P1.1.2). The first region can bind to the respective complementary position in primer extension products and can be extended by the polymerase template-dependent. In a reverse synthesis, the first region is copied by the polymerase. The second region (polynucleotide tail) is not copied by the polymerase. Said region can comprise nucleotide modifications that prevent a polymerase from using this region as a template (e.g. this region consists of 2′-O-alkyl modifications of nucleotides). Furthermore, a linker may be placed between the first segment and the second segment, which also prevents the polymerase from using the second segment as a template. The expected position of the stop of the polymerase during the synthesis of the strand complementary to the first region is called stop-1.1 here. The polynucleotide tail is not copied by the polymerase and thus remains single-stranded. It can be used to initiate the binding of controller 1.1 to the P1.1 Ext.

The first primer P1.1 can bind to the controller C1.1, wherein a complex is formed P1.1/C1.1 complex.

The second oligonucleotide primer (P2.1) comprises a first region (P2.1.1) and a second region (P2.1.2). The first region can bind to the respective complementary position in primer extension products and can be extended by the polymerase template-dependent. In a reverse synthesis, the first region is copied by the polymerase. The second region (polynucleotide tail) is not copied by the polymerase. This region may comprise nucleotide modifications that prevent a polymerase from using this region as a template (e.g. this region consists of 2′-O-alkyl modifications of nucleotides). Furthermore, a linker may be placed between the first segment and the second segment, which also prevents the polymerase from using the second segment as a template. The expected position of the stop of the polymerase during the synthesis of the strand complementary to the first region is called stop-2.1. The polynucleotide tail is not copied by the polymerase and remains single-stranded. It can be used to initiate the binding of controller 2.1 to the P2.1-Ext.

The first primer P2.1 can bind to the controller C2.1, wherein a complex is formed, the P2.1/C2.1 complex.

Particularly P1.1 does not bind to the controller C2.1 and P2.1 does not bind to C1.1.

Segment P1.1.1 of the first primer is identical to segment P1.1E5 of the first primer extension product. Segment P1.1.2 of the first primer is identical to segment P1.1E6 of the first primer extension product Segment P2.1.1 of the second primer is identical to segment P2.1E5 of the second primer extension product. Segment P2.1.2 of the second primer is identical to segment P2.1E6 of the second primer extension product.

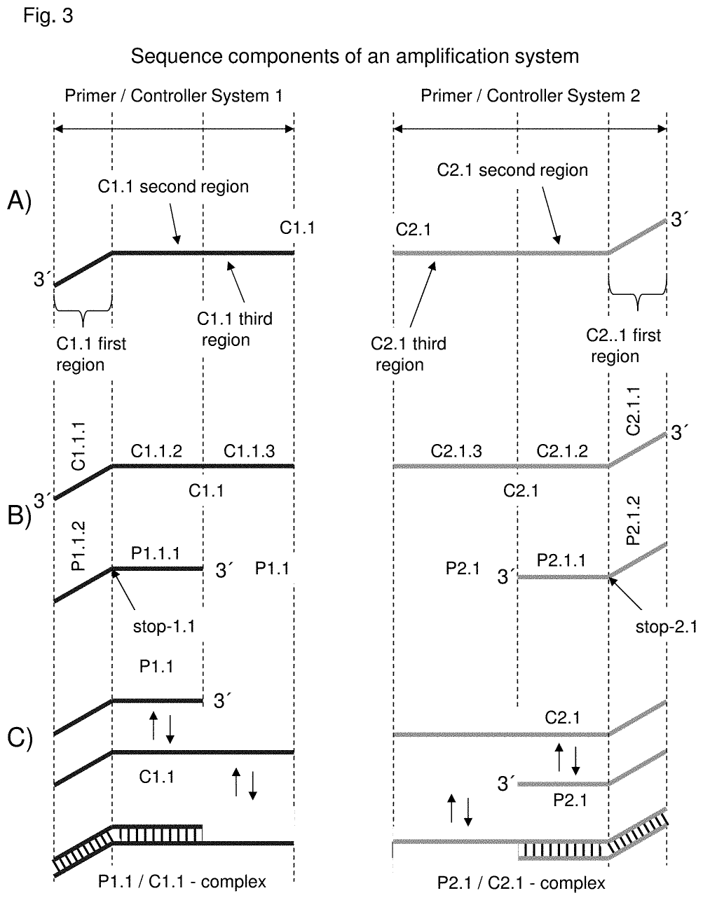

FIG. 3 shows schematically the topography of components in an embodiment of the invention.

The first controller oligonucleotide (C1.1) comprises a first region (C1.1.1), a second region (C1.1.2) and a third region (C1.1.3). The region C1.1.1 can bind to the P1.1.2 predominantly complementary. The region C1.1.2 can bind to P1.1.1 in a complementary manner. Region C1.1.3 can bind complementarily to the extension segment of the first segment synthesized by the polymerase (P1.1.E4).

The controller oligonucleotide (C1.1) comprises modifications that prevent a polymerase from using the first primer bound to the controller oligonucleotide as a template. In addition, the controller oligonucleotide comprises nucleotide modifications that prevent a polymerase from extending the primer using the controller as template. For example, C1.1.2 and C1.1.3 comprises several 2′-O-alkyl modifications of nucleotides. The first primer P1.1 can bind to the controller C1.1, wherein a complex is formed, the P1.1/C1.1 complex.

The second controller oligonucleotide (C2.1) comprises a first region (C2.1.1), a second region (C2.1.2) and a third region (C2.1.3). The region C2.1.1 can bind to the P2.1.2 predominantly complementary. The region C2.1.2 can bind to the P2.1.1 in a complementary manner. Region C2.1.3 can bind complementarily to the extension segment of the second primer synthesized by the polymerase (P2.1 E4).

The controller oligonucleotide (C2.1) comprises modifications that prevent a polymerase from using the second primer bound to the controller oligonucleotide as a template. In addition, the controller oligonucleotide comprises nucleotide modifications that prevent a polymerase from extending the primer using the controller as template. For example, C2.1.2 and C2.1.3 comprise several 2′-O-alkyl modifications of nucleotides. The second primer P21.1 can bind to the controller C2.1, wherein a complex is formed, the P2.1/C2.1 complex.

The interaction of the primers and controllers described above thus enables the assembly of specific controller-primer pairs. Primer 1.1 and Controller 1.1 form the primer-Controller System 1.1, and primer 2.1 and Controller 2.1 form the primer-Controller System 2.1.

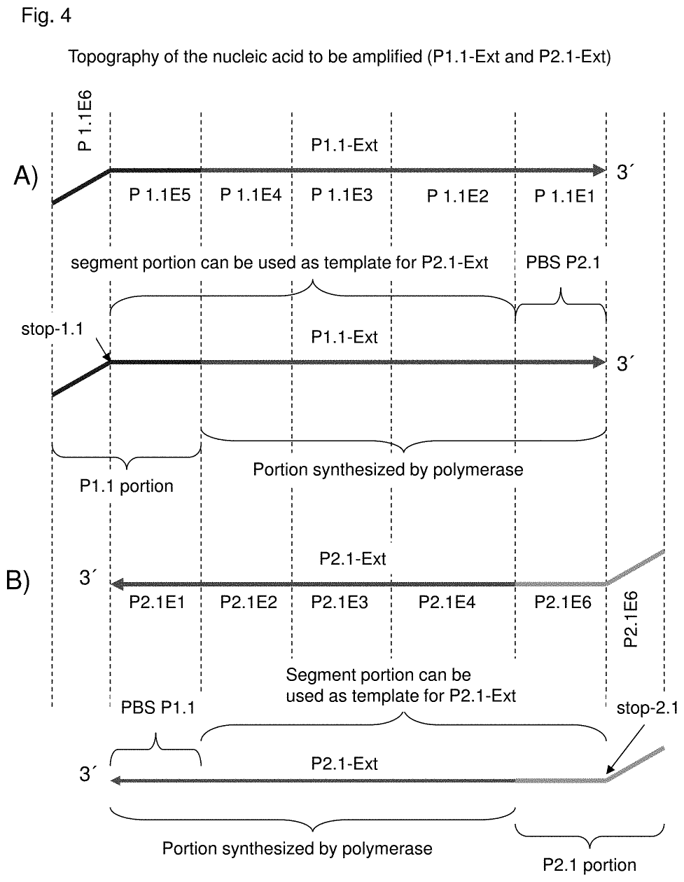

FIG. 4 A shows schematically the topography of the first synthesized primer extension product (also called primer extension product, P1.1-Ext). The P1.1 portion of the primer extension product P1.1 comprises the first region and the second region (with stop-1.1 modification and the polynucleotide tail 1.1.). The portions synthesized by the polymerase are located in the 3′ direction of the primer portion during template-dependent synthesis. These portions or segments serve as templates for the polymerase and as primer binding sites for primer 2.1 (PBS P2.1) during further synthesis steps. The stop 1.1 element of the primer P1.1 prevents the copying of the respective oligonucleotide tail. For individual primer extension products, the above arrangement of segments shows which segments are represented by the primer and which are synthesized by the polymerase.

FIG. 4 B shows schematically the topography of the second primer extension product (also called primer extension product, P2.1-Ext). The primer extension product 2.1 (P2.1-Ext) comprises the P2.1 portion with the first region and the second region (with stop-2.1 modification and the polynucleotide tail 2.1).

The portions synthesized by the polymerase are located in the 3′-direction of the primer portions during template-dependent synthesis. Said portions also serve as templates for the polymerase and as primer binding sites for the primer 1.1 (PBS P1.1) during further synthesis steps. The stop-2.1 element of P2.1 also prevents the respective oligonucleotide tail from being copied. For individual primer extension products, the above arrangement of segments shows which segments are represented by the primer and which are synthesized by the polymerase.

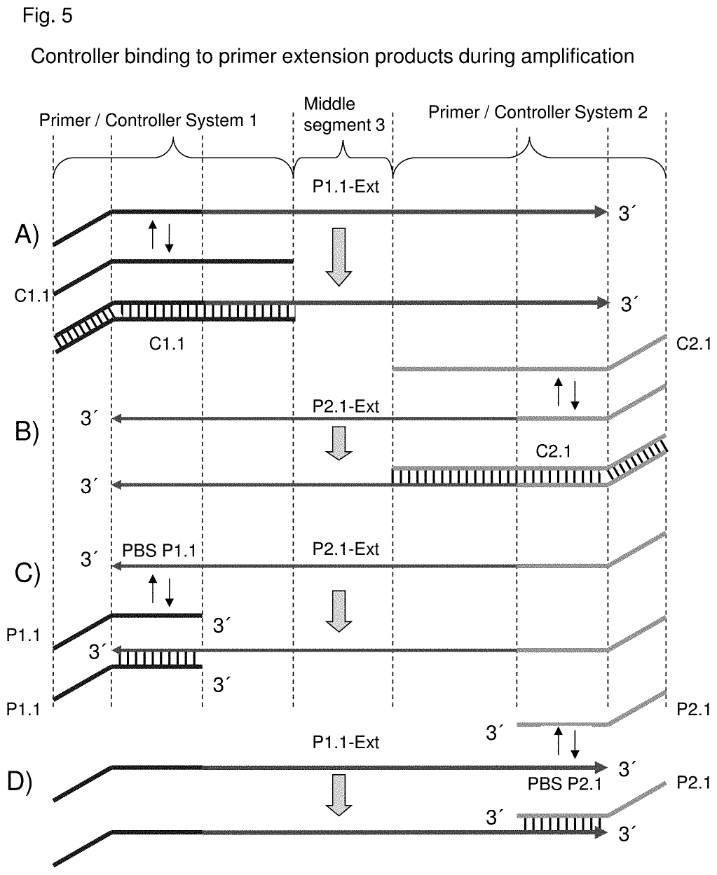

FIG. 5 schematically illustrates the controller binding and the primer binding to the primer extension products.

In A, controller C1.1 binds to the primer extension product P1.1-Ext. forming a double strand in the 5′ segment of the primer extension product P1.1-Ext.

In B, Controller C2.1 binds to the primer extension product P2.1-Ext. forming a double strand in the 5′ segment of P2.1-Ext. The middle segment of the respective primer extension product does not interact with the controllers.

In C, primer P1.1 binds to the primer extension product P2.1-Ext (which comprises a primer binding site for P1.1 in the 3′ segment) forming a double strand in the 3′ segment of the P2.1-Ext.

In D, primer P2.1 binds to the primer extension product P1.1-Ext (which comprises a primer binding site for P2.1 in the 3′ segment) to form a double strand in the 3′ segment of the P2.1-Ext.

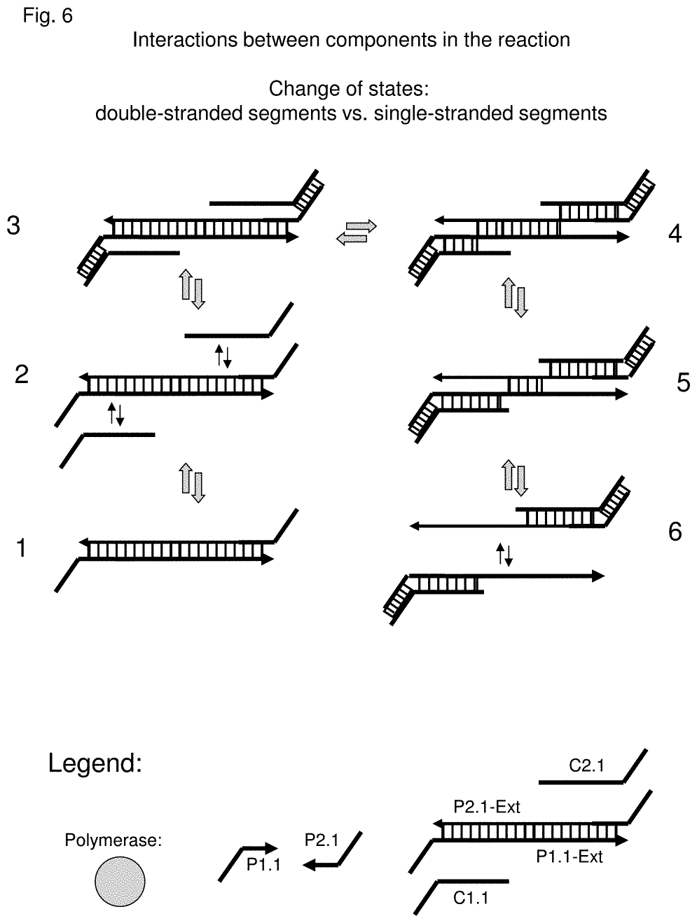

FIG. 6 schematically illustrates the interaction between the controllers (C1.1 and C2.1) and the primer extension products (P1.1-Ext. and P2.1-Ext.). In 1) the double-stranded complex comprising P1.1-Ext and P2.1-Ext is shown immediately after a primer extension reaction by a polymerase. Here, both primer extension products form a complementary double strand. In 2) the potential interaction positions for the two controllers (C1.1. and C2.1.) and the double-stranded complex comprising P1.1-Ext and P2.1-Ext are schematically shown. In 3) the initiation of specific binding from the controller to the respective specific polynucleotide tails of the primer extension products is shown, wherein C1.1 binds to the polynucleotide tail of P1.1-Ext and C2.1 binds to the polynucleotide tail of P2.1-Ext. In 4) a sequence-specific strand displacement by the respective controller is shown. C1.1 forms a double strand with parts of P1.1-Ext and C2.1 forms a double strand with parts of P2.1-Ext. In 5) a sequence-specific strand displacement by the respective controller is also shown schematically, wherein the segment in which P1.1-Ext and P2. 1-Ext are still bound to each other is shorter than in (4): Here C1.1 has designed a double strand with parts of P1.1-Ext and C2.1 a double strand with parts of P2.1-Ext, so that P1.1-Ext and P2.1-Ext remain bound to each other by the middle segment 3. The sequence segments of the middle region 3 are bound to each other in form of a complementary double strand. In 6) a dissociation or separation of P1.1-Ext and P2.1-Ext from each other is shown after a separation of the two strands in the middle segment 3. This results in the new complexes: P1.1-Ext/C1.1 and P2.1-Ext/C2.1 Both complexes respectively comprise a primer binding site for primers, which is present in single-stranded form.

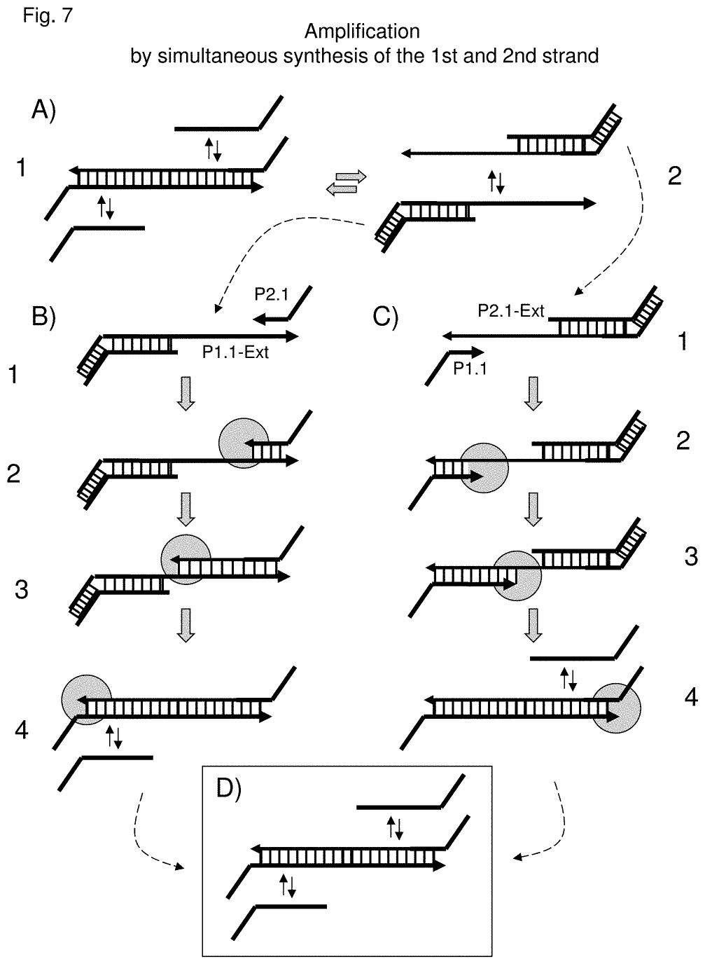

FIG. 7 shows schematically an amplification by the simultaneous synthesis of the first and second primer extension product (P1.1-Ext and P2.1-Ext). P1.1-Ext serves as a template for the polymerase-dependent synthesis of P2.1-Ext, using the second primer P2.1 to initiate the synthesis by the polymerase. P2.1-Ext serves as a template for the polymerase-dependent synthesis of P1.1-Ext, using the first primer P1.1 to initiate the synthesis by the polymerase. The respective separation of P1.1-Ext and P2.1-Ext after the respective synthesis phase takes place with the participation of both controller oligonucleotides (C1.1) and (C2.1). An exponential amplification of both primer extension products P1.1-Ext and P2.1-Ext results.

FIG. 7 A illustrates an interaction between the controllers C1.1 and C2.1 and the double-stranded complex comprising P1.1-Ext and P2.1-Ext (A1) and a dissociation or separation of P1.1-Ext and P2.1-Ext from each other after separation of the two strands in the middle segment 3. This results in the new complexes: P1.1-Ext/C1.1 and P2.1-Ext/C2.1 Both complexes comprise respectively a primer binding site for primers, which is located in single stranded form. In B) and C) respectively the use of the complexes P1.1-Ext/C1.1 (FIG. 7B) and P2.1-Ext/C2.1 (FIG. 7C) as templates for the synthesis is shown. These include the primer binding to the respective PBS (1), the polymerase binding to the primer/PBS complex (2), the synthesis of the complementary strand by primer extension at the respective template (3), the synthesis in the single-stranded region is shown, and the completion of the synthesis of the complementary strand by primer extension at the respective template while simultaneously displacing the respective controller oligonucleotide (4), the strand displacement being mediated by the polymerase.

The individual embodiments of the amplification of the method according to the invention are described below. The synthesis of the respective P1.1-Ext or P2.1-Ext can start from a start nucleic acid chain. The start nucleic acid is provided at the beginning of the reaction. First, the embodiment is shown in which M1.1 is used as the start nucleic acid chain.

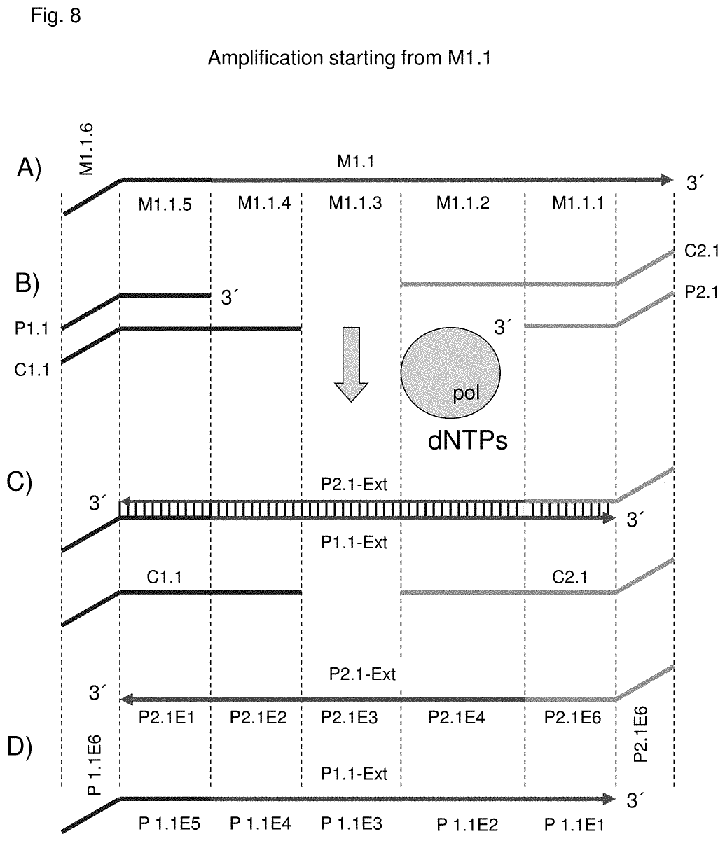

FIGS. 8-12 show schematically the amplification starting from M1.1.

In summary, a synthesis can take place using a start nucleic acid chain (M1.1) (8A), a polymerase, dNTPs and respective P1.1/C1.1 and P2.1/C2.1 (8B), resulting in an exponential increase of P1.1-Ext and P2.1-Ext (C).

The start nucleic acid chain (M1.1) (FIG. 8A) is provided at the beginning of the amplification and comprises the following segments (in 5′-3′ order): M1.1.6, M1.1.5, M1.1.4, M1.1.3, M1.1.2, M1.1. 1, wherein said segments are essentially identical to desired corresponding segments of P1.1-Ext (in 5′-3′ order): P1.1E6, P1.1E5, P1.1E4, P1.1E3, P1.1E2, P1.1E1 (FIG. 8D).

The provision of such a start nucleic acid chain M1.1 is known to a skilled person. It can be prepared by a primer extension reaction using P1.1 and a nucleic acid strand comprising a target sequence (here called target sequence 1) (FIG. 9). A P1.1 is hybridized to a 3′ segment of a target sequence (FIG. 9B) so that a primer extension reaction can take place. Using a polymerase and respective substrates (dNTPs), such a primer can be extended using a nucleic acid strand comprising the target sequence, so that target sequence 1 serves as template and is copied during the procedure and a start nucleic acid chain M1.1 is synthesized (FIG. 9C). By separating (FIG. 9D) the synthesized M1.1 from the nucleic acid strand (comprising the target sequence), the start nucleic acid chain M1.1 is converted into single-stranded form. Such a start nucleic acid chain can be used for the amplification of P1.1-Ext and P2.1-Ext (FIG. 9E).

The use of a nucleic acid strand comprising a target sequence for a primer extension reaction is known to a skilled person. Such a primer extension reaction can be performed once to provide a starting nucleic acid chain or the procedure can be repeated cyclically, i.e. several times, wherein one copy of M1.1 can be provided for each target sequence using P1.1. The separation of M1.1 from the nucleic acid strand comprising the target sequence and its transformation into a single-stranded state is also known to a skilled person: this can take place, for example, through temperature, which leads to the separation of formed strands. Generally, a temperature between 85° C. and 105° C. can be used. Alternatively, a polymerase-dependent strand displacement can be used to separate M1.1 from the strand comprising the target sequence. A so-called bumper primer is used, which is hybridized in the 3′ direction of the nucleic acid strand comprising the target sequence, so that M1.1 is displaced by polymerase during this synthesis. Alkaline strand separation is also possible, using for example 0.1 mol NaOH. The use of a first primer in the preparation of M1.1 is important: this introduces a region in the 5′ region of the start nucleic acid chain which cannot be copied by the polymerase. In this case, this is the second region of the first primer. Thus, segment M1.1.6 corresponds to segment P1.1.2 and segment M1.1.5 corresponds to segment P1.1.1. Segments M1.1.4 to M1.1.1 are given by the composition of the target sequence when creating M1.1. FIG. 10 schematically summarizes which segments of the target sequence serve as templates and are used in the creation of M1.1: The target sequence 1 comprises the following segments (5′-3′ arrangement): TS1.5, TS1.4, TS1.3, TS1.2, TS1.1. Said segments can be flanked at the 3′ end by segment TS1.X and at the 5′ end by segment TS1.Y. segment TS1.1 can predominantly bind complementarily to the first region of the first primer (P1.1.1), so that the polymerase can copy the target sequence starting from the 3′-end of the hybridized primer, wherein M1.1 is generated. The choice of such a target sequence is known to a skilled person. For example, a genomic DNA or RNA may comprise such a target sequence or artificially produced sequences may also comprise such a target sequence. FIG. 10 summarizes which sequence segments of a target sequence (FIG. 10A) lead to which segments in the products derived from such a target sequence (comprising M1.1 (FIG. 10C) and P1.1-Ext and P2.1-Ext (FIG. 10E). Based on general rules of reverse complement, a skilled person can calculate the desired composition of respective components. The target sequence can be the starting point of said calculation as well as intermediate products (M1.1) or final products (P1.1-Ext and P2.1-Ext). The respectively complementary regions of primers and controllers can also be defined to support exponential amplification.

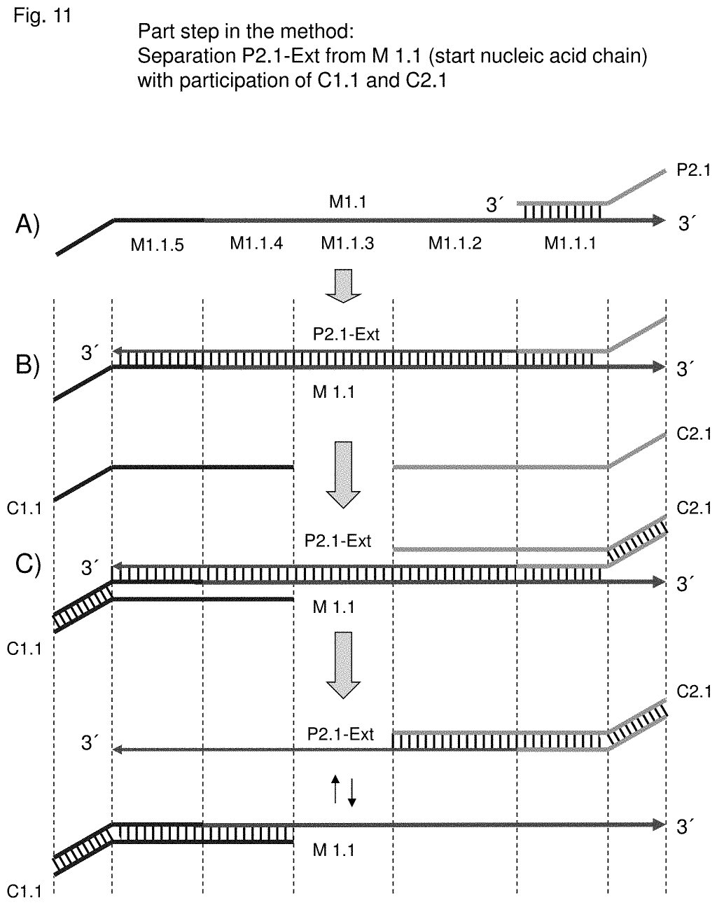

FIG. 11 shows the following: The amplification starts from M1.1, wherein first a primer extension reaction is performed (using a polymerase and dNTPs) by hybridizing P2.1 to the essentially complementary primer binding site of M1.1 (segment M1.1.1), in which M1.1 acts as a template. Due to the nature of the M1.1 provided, the polymerase synthesis is stopped at the Stop-1.1 position of M1.1. The product synthesized by the primer extension reaction corresponds to the respective P2.1 Ext. (FIG. 11 B). With the participation of controller C1.1 and C2.1, the strands (M1.1 and P2.1-Ext) are separated under reaction conditions so that primer binding sites are available for renewed primer binding. Primers can now bind to these single-stranded segments again and be extended by the polymerase.

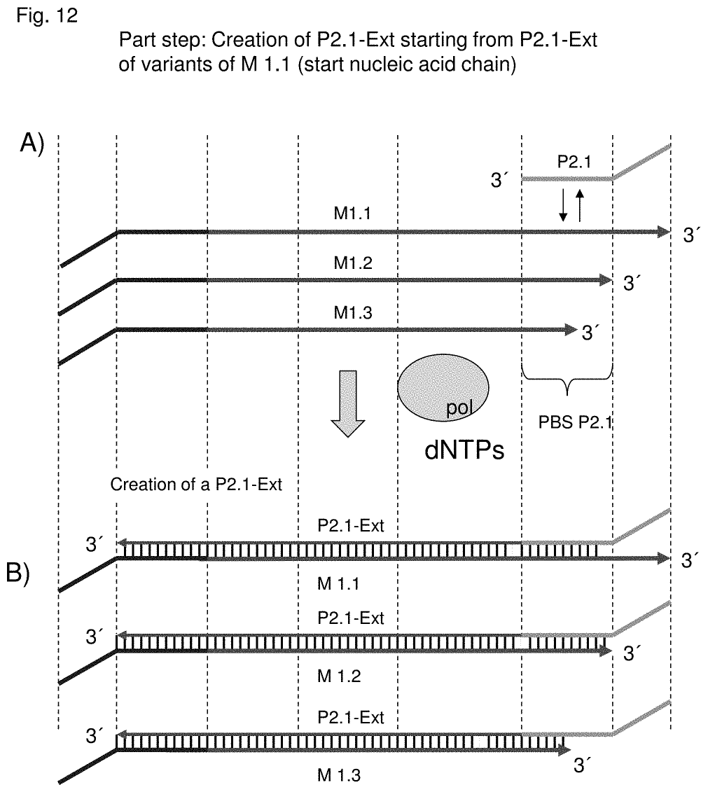

FIG. 12 shows that nucleic acid chains that do not comprise exact complementarity to P2.1.1 or that differ in the length of the 3′ segment (M1.1; M1.2; M1.3) can also be used as start nucleic acid chains. Crucially, P2.1 can predominantly bind specifically to segment M1.1.1 and can be extended by the polymerase, resulting in P2.1-Ext (FIG. 11 B).

FIG. 13 summarizes that M2.1 can also be used in a similar way as a starting nucleic acid chain.

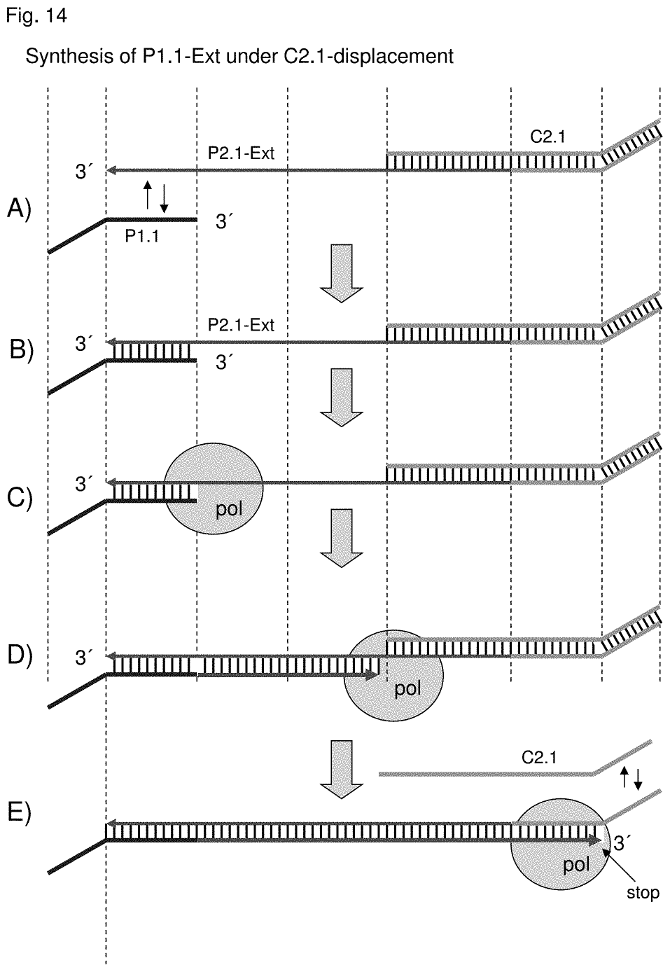

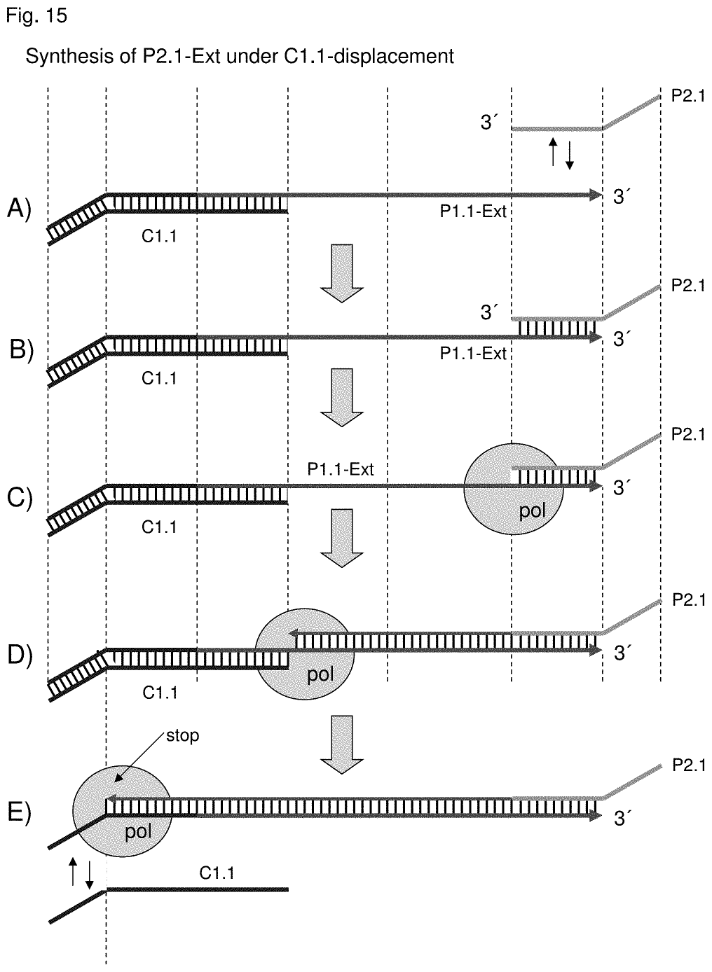

FIG. 14-15 shows schematically the primer extension while simultaneously displacing the respective controller from its binding with the primer extension product.

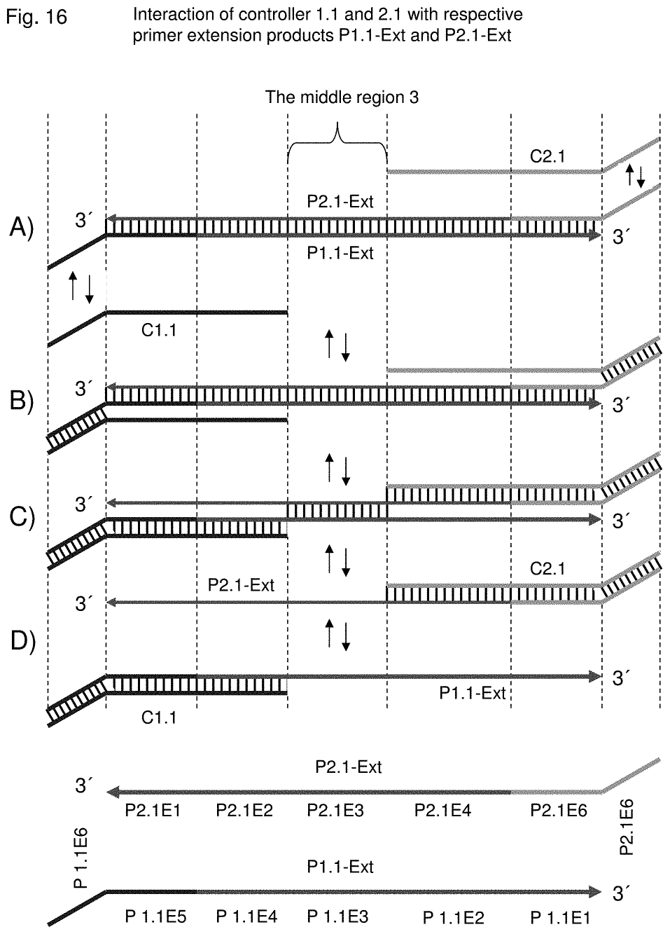

FIG. 16 illustrates the interaction of C1.1 and C2.1 with the complex of P1.1-Ext and P2.1-Ext. Initial binding of controllers takes place via respective polynucleotide tails (FIG. 16 B), which can lead to double strand formation (C1.1/P1.1-Ext and C2.1/P2.1-Ext) if the sequence of synthesized strands and controller strands match. P1.1-Ext and P2.1-Ext remain bound complementarily in the middle region (3) (FIG. 16 C). Under respective reaction conditions, this complex can dissociate into single strands in the middle region, which leads to the separation of P1.1-Ext and P2.1-Ext (FIG. 16 D).

The length of this middle region can be between 0 and 60 nucleotides, especially between 1 and 40 nucleotides, particularly between 5 and 30 nucleotides. In certain embodiments of the invention, this middle region is not bound by either of the two controllers. Segments P1.1E3 and P2.1E3 correspond to said middle region 3. The corresponding segment in the target sequence 1 is TS 1.3; in M1.1 it is M1.1.3.

Especially reaction conditions are used which do not allow spontaneous separation of double strands comprising P1.1-Ext and P2.1-Ext in the absence of at least one controller.

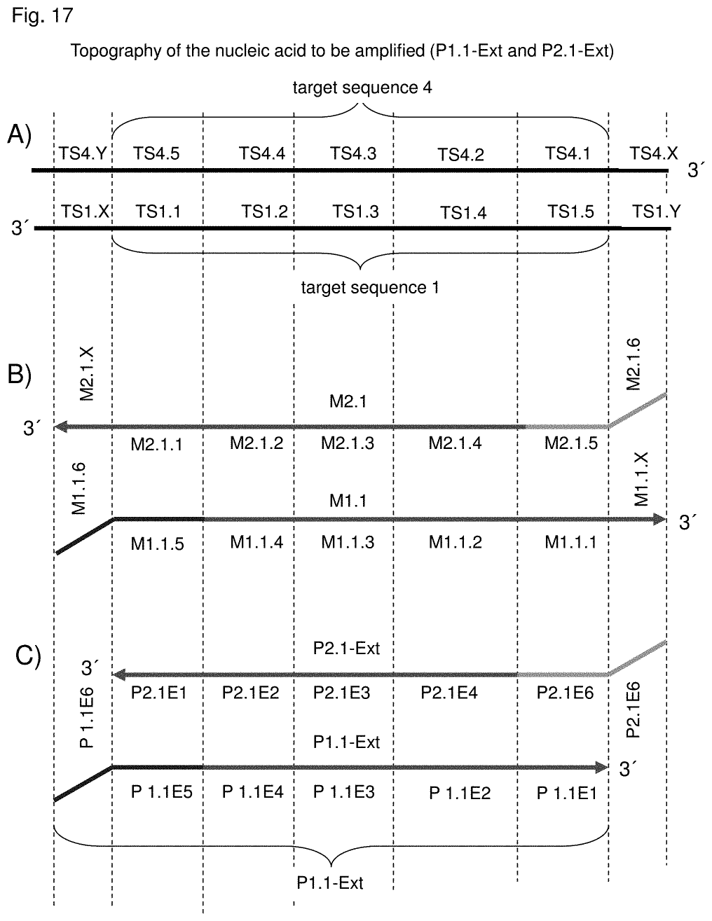

FIGS. 17-19 show schematically different embodiments of topographies of single target sequences and derived corresponding strands of start nucleic acid chains and resulting primer extension products (P1.1-Ext and P2.1-Ext).

FIG. 20 shows schematically the generation of the start nucleic acid chain M2.1 using target sequence 4.

### Example 3: Creation of a Start Nucleic Acid Chain by PCR Starting from Single-Stranded Nucleic Acid Chains

A synthesis of start nucleic acid chains (M1.1 and M 2.1) took place by means of a PCR starting from a single-stranded nucleic acid chain comprising the target sequences. Primers with a structure that was also used for the first and second primers of the subsequent amplification were used. The starting nucleic acid chains obtained (M1.1 and M 2.1) comprised a target sequence or a sequence complementary to the target sequence, as well as a segment M1.1.6 or M2.1.6 (overhang comprising modified nucleotides shown in FIG. 9 A-D and FIG. 20 A-D)

### Templates:

T2C-300-3001 (SEQ ID NO 27):
5′- TACCTGTATTCC TT GCCTGTCCAG GGATCTGCTC
TTACAGATTA AAA TATTAGCCCAGAG GCGATGTCTC TCATGATACA
GGTATTTTG AC-3′
T2C-300-3002 (SEQ ID NO 28):
5′- TACCTGTATTCC TT GCCTGTCCAG GGATCTGCTC
TTACAGATTA CTTAGAG CTTAGAG TATTAGCCCAGAG
GCGATGTCTC TCATGATACA GGTATTTTG AC-3′
(Templates were used in PCR at a concentration
of approx. 0.1 nmol/l).

### PCR Batch:

First PCR primer (0.5 μmol/l) (P1F5G2-1001-101 K)
(SEQ ID NO 29):
5′- CACAATCATCAATAC TTTTTT GTCAAAATA
[CUCU GAUGCUCU] 1 GT CAAAATACCTGT

The underlined sequence part (position 1-14 from the 3′ end) corresponds to the respective first region of a primer and can hybridize sequence-specifically to a template and be extended by the polymerase.

Second PCR primer (0.5 μmol/l) (P1F5-001-2203-7 )
(SEQ ID NO 30):
5′- [CUCU GAUGCUUC] 1 TACCTGTATTCCTTGC
A = 2′-deoxy adenosine, C = 2′-deoxy cytosine,
G = 2′-deoxy guanosine, T = 2′-deoxy thymidine
(thymidine)

The underlined sequence part (position 1-16 from the 3′ end) corresponds to the respective first region of a primer and can hybridize sequence-specifically to a template and be extended by the polymerase.

The bracketed region [CUCU GAUGCUCU] of the first PCR primer and the bracketed region [CUCU GAUGCUUC] of the second PCR primer comprised modifications (2′-O-methyl nucleotides: 2′-O-Me A (2′-O-methyl adenosines), 2′-O-Me G (2′-O-methyl guanosines), 2′-O-Me C (2′-O-methyl cytosines), 2′-O-Me U (2′-O-methyl uridines). 1=C3-linker. These sequence segments correspond to the respective second region of a primer.

Taq Polymerase (NEB) (used in a dilution of 1:100 starting from stock solution)
1× Isothermal Buffer (New England Biolabs, catalog #B0537S; in single concentration the buffer contains: 20 mM Tris-HCl; 10 mM (NH4)2SO4; 50 mM KCl; 2 mM MgSO4; 0.1% Tween®20; pH 8.8@25° C.); dNTP (dATP, dCTP, dUTP, dGTP), each 200 μmol/l;
EvaGreen dye (Jena Biosciences) in 1:50 dilution.

The PCR reaction was performed over 25 cycles. One cycle comprised 1 min at 54° C., 1 min at 65° C. A final extension was performed for 10 min at 68° C. For products of the reaction starting from the template T2C-300-3001 a Tm of about 80° C. was measured. For the products of the reaction starting from template T2C-300-3002 a Tm of approx. 81° C. was measured.

As a result of the PCR reaction the following products were obtained (start nucleic acid chains M1.1 and M2.1):

Mixture 7 (using template T2C-300-3001)
Product: M1.1-T2C-300-3001 (Start nucleic acid
chain M1.1) (M1.1.3 = 3 nucleotides)
(SEQ ID NO 31)
5′-CACAATCATCAATAC TTTTTT GTCAAAATA [CUCU
GAUGCUCU] 1 GT CAAAATACC TGTATCATGA GAGACATCGC
CTCTGGGCTAATA TTT TAATCTGTAA GAGCAGATCC
CTGGACAGGC AA GGAATACAGGTA 3′
Product: M2.1-T2C-300-3001 (Start nucleic acid
chain M2.1) (M2.1.3 = 3 nucleotides)
(SEQ ID NO 32)
5′- [CUCU GAUGCUUC] 1 TACCTGTATTCC TT
GCCTGTCCAG GGATCTGCTC TTACAGATTA AAA TATTAGCCCAGAG
GCGATGTCTC TCATGATACA GGTATTTTG AC-3′
Mixture 8 (using template T2C-300-3002)
Product: M1.1-T2C-300-3002 (Start nucleic acid
chain M1.1) (M1.1.3 = 14 nucleotides)
(SEQ ID NO 33)
5′-CACAATCATCAATAC TTTTTT GTCAAAATA [CUCU
GAUGCUCU] 1 GT CAAAATACC TGTATCATGA GAGACATCGC
CTCTGGGCTAATA CTCTAAG CTCTAAG TAATCTGTAA
GAGCAGATCC CTGGACAGGC AA GGAATACAGGTA 3′
Product: M2.1-T2C-300-3002 (Start nucleic acid
chain M2.1) (M2.1.3 = 14 nucleotides)
(SEQ ID NO 34)
5′- [CUCU GAUGCUUC] 1 TACCTGTATTCC TT GCCTGTCCAG
GGATCTGCTC TTACAGATTA CTTAGAG CTTAGAG
TATTAGCCCAGAG GCGATGTCTC TCATGATACA GGTATTTTG
AC-3′
A = 2′-deoxy adenosine, C = 2′-deoxy cytosine,
G = 2′-deoxy guanosine, T = 2′-deoxy thymidine
(thymidine)

The regions in brackets (M 1.1.6) [CUCU GAUGCUCU] of the respective start nucleic acid chain M 1.1 and the region in brackets (M 2.1.6) [CUCU GAUGCUUC] of the respective start nucleic acid chain M 2. 1 comprise modifications (2′-O-methyl nucleotides: 2′-O-Me A (2′-O-methyl adenosines), 2′-O-Me G (2′-O-methyl guanosines), 2′-O-Me C (2′-O-methyl cytosines), 2′-O-Me U (2′-O-methyl uridines). 1=C3-linker. These sequence segments correspond to a respective overhang.

The resulting PCR products correspond to the start nucleic acid chain (M1.1 and M2.1) and comprise either the target sequence or its complementary sequence, respective overhangs (M 1.1.6 or M 2.1.6) as well as primer binding sites to which primers can bind during amplification. These products were used in the amplification as a mixture of both primer extension products (example 4).

### Example 4: Exponential Amplification of Amplification Fragments Starting from Start Nucleic Acid Chain

The amplification was performed using two sequence-specific controller oligonucleotides and two sequence-specific primers.

Two variants of primers were used. The first variant (primer mix 1) comprised primers with a so-called “basic structure” (“minimal structure”), which included a first segment and a second region. No additional sequence segments were used. Primer-Mix 2 comprised primers with a structure that used additional sequence segments in addition to a first and a second region.

Both primer mixes were used with the same controller oligonucleotides.

### Oligonucleotides Used:

### Primer-Mix 1

The first oligonucleotide primer:
P1F5G2-1001-103 TMR
(SEQ ID NO 35)
5′-TMR [CUCU GAUGCUCU] 1 GT CAAAATACC T
The second oligonucleotide primer: P1F5-001-1016
(SEQ ID NO 36)
5′ [cucu gaugcuuc] 1 tacctgtattcc
A = 2′-deoxy adenosine; C = 2′-deoxy cytosine,
G = 2′-deoxy guanosine, T = 2′-deoxy thymidine
(thymidine)

The underlined sequence part (position 1-12 from the 3′-end) corresponds to the first region of a primer and can hybridize sequence specifically to the respective regions of a start nucleic acid chain and be extended by the polymerase. The first oligonucleotide primer binds to M2.1, the second oligonucleotide primer binds to M1.1.

The region in brackets [CUCU GAUGCUCU] of the first region of the first oligonucleotide primer and the region in brackets [CUCU GAUGCUUC] of the second oligonucleotide primer comprise modifications (2′-O-methyl nucleotides: 2′-O-Me A (2′-O-methyl adenosines), 2′-O-Me G (2′-O-methyl guanosines), 2′-O-Me C (2′-O-methyl cytosines), 2′-O-Me U (2′-O-methyl uridines). 1=C3-linker. Said sequence segments correspond to the respective second region of an oligonucleotide primer. The first primer comprised a 5′-TMR modification.

### Primer Mix 2

The first oligonucleotide primer:
P1F5G2-1001-103
(SEQ ID NO 37)
5′- CACAATCATCAATAC TTTTTT GTCAAAATA
[CUCU GAUGCUCU] 1 GT CAAAATACCT
The second oligonucleotide primer: P1F5-50-AE2051
(SEQ ID NO 1)
5′- CACAATCATCAATAC TTTTTT TACCTGTAT [CUCU
GAUGCUUC] 1 TACCTGTATTCC
A = 2′-deoxy adenosine, C = 2′-deoxy cytosine,
G = 2′-deoxy guanosine, T = 2′-deoxy thymidine
(thymidine)

The underlined sequence part (position 1-12 from the 3′ end) corresponds to the respective first region of a primer and can hybridize sequence-specifically to a template and be extended by the polymerase.

The bracketed region

[CUCU GAUGCUCU]

of the first oligonucleotide primer and the bracketed region

[CUCU GAUGCUUC]

of the second oligonucleotide primer comprised modifications (2′-O-methyl nucleotides: 2′-O-Me A (2′-O-methyl adenosines), 2′-O-Me G (2′-O-methyl guanosines), 2′-O-Me C (2′-O-methyl cytosines), 2′-O-Me U (2′-O-methyl uridines). 1=C3-linker. These sequence segments correspond to the respective second region of an oligonucleotide primer.

These oligonucleotide primers comprise the first region (positions 1-12 from the 3′-end), the second region (C3-linker, as well as positions 13-24 from the 3′-end), as well as one segment each with an additional sequence variant P1 (positions 25-54 from the 3′-end). The first region and the second region are necessary for the execution of a specific amplification and can be summarized as “basic structure of the first primer” or “minimal structure of the first primer”. The additional sequence variant P1 is an example of additional segments which can be integrated at the first oligonucleotide primer. A C3 modification and the second region prevent the continuation of the synthesis at positions 25-54 during a synthesis of the second primer extension product.

Following controller oligonucleotides were used:

The first controller oligonucleotide:
C2xCF-P1-103-101
(SEQ ID NO 38)
5′ [UAUUAGCCCAGAGGCGAUGUCUCUCAUGAU ACA GGUAUU]
TTG AC AGAGCATC AGAGAG X 3′
The second controller oligonucleotide:
CF5-1001-11-6
(SEQ ID NO 25)
5′ [A GGACUACUUC UAAUCUGUAA GAGCAGAUCC
CUGGACAGGC AA GGAAUACAG] GTA GAAGCATC AGAG X 3′
A = 2′-deoxy adenosine, C = 2′-deoxy cytosine,
G = 2′-deoxy guanosine, T = 2′-deoxy thymidine
(thymidine)

The region in parentheses

[UAUUAGCCCAGAGGGCGAUGUCUCUCUCAUGAU ACA GGUAUU]

of the first controller oligonucleotide and the region in parentheses

[A GGACUACUUC UAAUCUGUAA GAGCAGAUCC CUGGACAGGC
AA GGAAUACAG]

of the second controller oligonucleotide comprised modifications (2′-O-methyl nucleotides: 2′-O-Me A (2′-O-methyl adenosines), 2′-O-Me G (2′-O-methyl guanosines), 2′-O-Me C (2′-O-methyl cytosines), 2′-O-Me U (2′-O-methyl uridines). X=3-phosphate group.

No auxiliary oligonucleotides (e.g. block oligonucleotides) were used.

### The Reaction was Performed Under the Following Conditions:

The first and second primer were used in concentrations of 4 μmol/l respectively. The first and second controller oligonucleotide were used in concentrations of 2 μmol/l respectively.

Mixtures with DNA templates (Start nucleic acid chain M1.1 and M2.1) from example 3 (mixture 7 and 8) were used individually diluted 100-fold (1:600 v:v), 1000-fold (1:6000 v:v) and 60,000-fold (1:10,000 v:v) respectively.

In a number of batches a primer mix 1 with both controller oligonucleotides was used. In another batch a primer mix 2 with both controller oligonucleotides was used. The respective concentrations of M1.1 and M1.2, as well as the concentration of BST polymerase are given (see figure description).

The further reaction conditions were:
1× Isothermal Buffer (New England Biolabs, catalog #B0537S; in single concentration the buffer contains: 20 mM Tris-HC; 10 mM (NH4)2SO4; 50 mM KCl; 2 mM MgSO4; 0.1% Tween®20; pH 8.8@25° C.); dNTP (dATP, dCTP, dUTP, dGTP), each 200 μmol/l;
EvaGreen dye (Jena Biosciences) in 1:50 dilution.

The amplification took place using BST 2.0 polymerase (stock solution: Bst 2.0 WarmStart 120,000 units/ml, NEB) according to the manufacturer's instructions. The BST polymerase was present in the reaction mixture in 120-fold (1:120) or 1200-fold (1:1200) dilution of the polymerase stock solution. See labeling of figures).

The thermal reaction conditions were as follows:
Initial phase at 65° C. for 2 minutes.
60 cycles with the following parameters
   Phase at 50° C. for 2 min.
   Phase at 65° C. for 2 min.
Denaturation at 95° C. for 3 minutes.
Recording of a melting curve.

### Results and Evaluation:

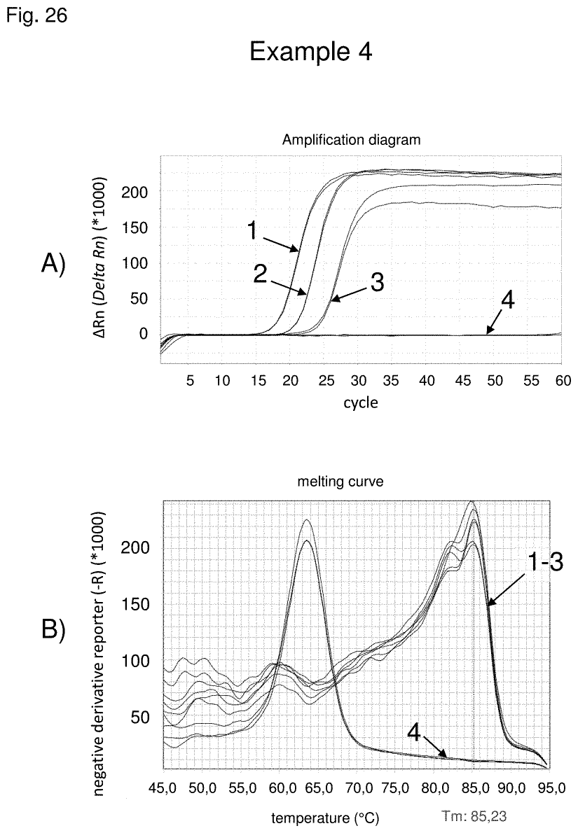

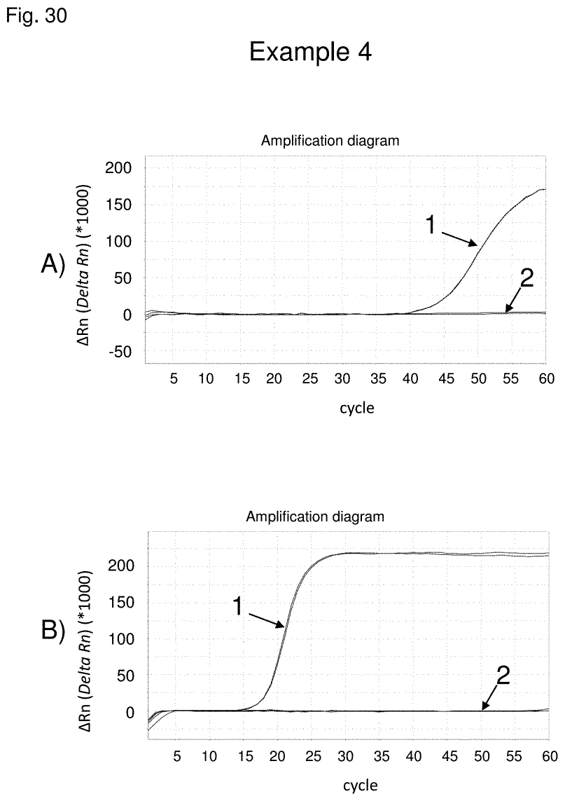

The amplification took place using Start nucleic acid chain M1.1 and M 2.1, prepared in example 3. The amplification was performed in batches with primer mix 1 as well as with primer mix 2. The reaction kinetics (observed by time of signal rise) of the amplification was observed. It was shown that the time of the visible signal increase depends on the input quantity of the start nucleic acid chain (FIGS. 26-29) and also on the quantity of polymerase used in the reaction mixture (1:120 or 1:1200) (FIG. 30). The melting temperature of obtained amplification products was above 80° C. Double-stranded products with such melting points are sufficiently stable under the reaction conditions used in the absence of controller oligonucleotides. Only the presence of controller oligonucleotides leads to a separation of synthesized strands so that amplification can take place.

The primers used in primer mix 1 (FIGS. 2 and 3) each comprised a first (P1.1.1 and P2.1.1) as well as a second region (P1.1.2 and P2.1.2). The used controllers each comprised a respective first (C1.1.1 and C 2.1.1), second (C1.1.2 and C 2.1.2) as well as third (C1.1.3 and C 2.1.3) regions. This shows that oligonucleotides with such structures were sufficient to perform the method.

The primers used in primer mix 2 comprised a first and a second region as well as additional sequence segments, which were located at the 5′ primer terminus. These structures do not participate in the amplification, but do not interfere with the amplification process. This shows that additional sequences can be introduced at the primer.

In comparison to example 2, no auxiliary oligonucleotides (block oligonucleotides) were used during amplification in this example 4. Hereby, a certain embodiment was shown: Use of primer—excess in relation to the concentration of controller oligonucleotides while using cyclic temperature conditions (50° C. and 65° C.).

### In Detail:

Analysis of Amplification Fragments after Exponential Propagation Starting from a Start Nucleic Acid Chain

The following techniques were used to analyze the amplification reaction and to evaluate the resulting amplification products:
Fluorescence signal of an intercalating dye (EvaGreen)
Melting curve analysis of the resulting amplification products.

FIG. 26-29 show the influence of the start nucleic acid chain used on the course of the reaction.

FIG. 26 (A) shows a typical course of the EvaGreen fluorescence signal during the amplification reaction. On the Y-axis the change of the fluorescence signal of the EvaGreen dye and on the X-axis the reaction time (as cycle number) is plotted. The increase of the fluorescence signal indicates that amplification products have been formed. At the start of the reaction the batches contained 600-fold (arrow 1), 6000-fold (arrow 2) and 60000-fold (arrow 3) diluted batches from example 3 with mixture 7 (M 1.1-T2C-300-3001 und M 2.1-T2C-300-3001). Arrow 4 shows the course of a negative control, here H₂O was used in the PCR to generate fragments instead of template. No amplification products were generated in the negative control. The amplification batch was carried out with primer mix 2 and respective controller oligonucleotide. The BST polymerase was used in a 1:120 dilution of the stock solution.

FIG. 26 (B) shows the melting curves belonging to the batches. The Y-axis shows the derivative of the fluorescence signal and the X-axis the temperature. A peak is visible, indicating that a uniform amplification product has been formed. The peaks marked with 1-3 belong to the batches that were prepared at the start of reaction mixture 7 (M 1.1-T2C-300-3001 und M 2.1-T2C-300-3001) in 600-fold, 6000- and 60000-fold dilutions. The curves marked with 4 comprise no product peak and belong to the negative control.

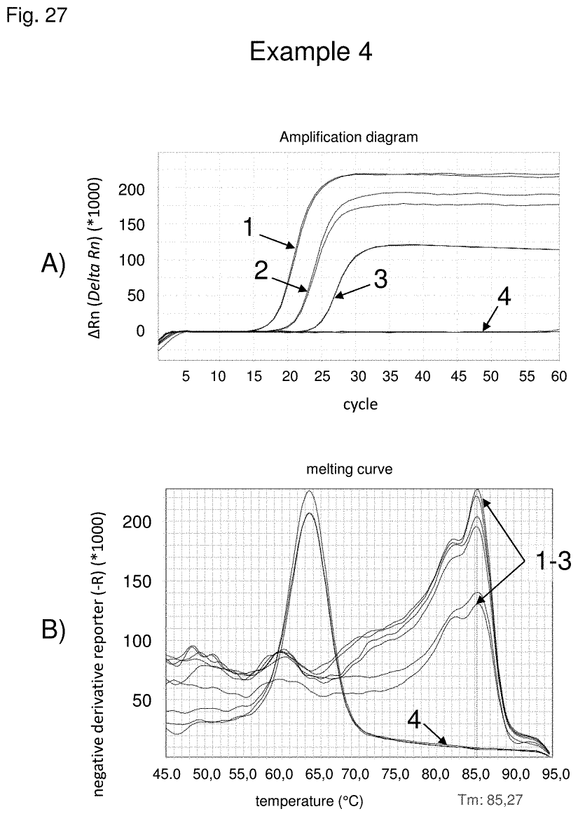

FIG. 27 (A) shows a typical course of the EvaGreen fluorescence signal during the amplification reaction. The Y-axis shows the change in the EvaGreen fluorescence signal and the X-axis shows the reaction time (as cycle number). The increase of the fluorescence signal indicates that amplification products have been formed. At the start of the reaction the batches contained 600-fold (arrow 1), 6000-fold (arrow 2) and 60000-fold (arrow 3) diluted batches from example 3 with mixture 8 (M 1.1-T2C-300-3002 und M 2.1-T2C-300-3002). Arrow 4 shows the course of a negative control, here H₂O was used in the PCR to generate fragments instead of template. No amplification products were generated in the negative control. The amplification batch was performed with primer mix 2 and respective oligonucleotide primers. The BST polymerase was used in a 1:120 dilution of the stock solution.

FIG. 27 (B) shows the melting curves belonging to the batches. The Y-axis shows the derivative of the fluorescence signal and the X-axis the temperature. A peak is visible, indicating that a uniform amplification product has been formed. The peaks marked with 1-3 belong to the batches that were prepared at the start of reaction mixture 8 (M 1.1-T2C-300-3002 und M 2.1-T2C-300-3002) in 600-fold, 6000- and 60000-fold dilutions. The curves marked with 4 comprise no product peak and belong to the negative control.

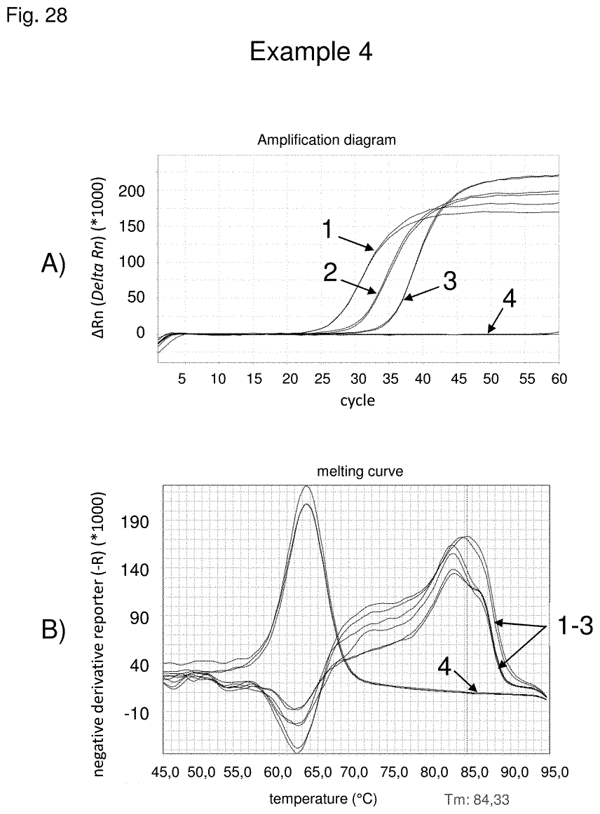

FIG. 28 (A) shows a typical course of the EvaGreen fluorescence signal during the amplification reaction. The Y-axis shows the change in the EvaGreen fluorescence signal and the X-axis shows the reaction time (as cycle number). The increase of the fluorescence signal indicates that amplification products have been formed. At the start of the reaction the batches contained 600-fold (arrow 1), 6000-fold (arrow 2) and 60000-fold (arrow 3) diluted batches from example 3 with mixture 7 (M 1.1-T2C-300-3001 und M 2.1-T2C-300-3001). Arrow 4 shows the course of a negative control, here H₂O was used in the PCR to generate fragments instead of template. No amplification products were generated in the negative control. The amplification batch was performed with primer mix 1 and respective oligonucleotide primers. The BST polymerase was used in 1 to 120-fold dilution of the stock solution.

FIG. 28 (B) shows the melting curves belonging to the batches. The Y-axis shows the derivative of the fluorescence signal and the X-axis the temperature. A peak is visible, indicating that a uniform amplification product has been formed. The peaks marked with 1-3 belong to the batches that were prepared at the start of reaction mixture 7 (M 1.1-T2C-300-3001 und M 2.1-T2C-300-3001) in 600-fold, 6000- and 60000-fold dilutions. The curves marked with 4 comprise no product peak and belong to the negative control.

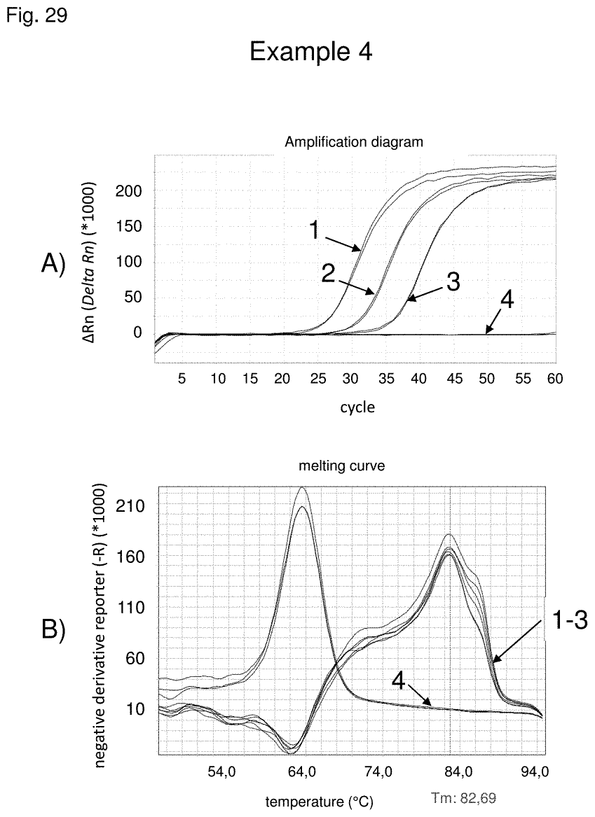

FIG. 29 (A) shows a typical course of the EvaGreen fluorescence signal during the amplification reaction. The Y-axis shows the change in the EvaGreen fluorescence signal and the X-axis shows the reaction time (as cycle number). The increase of the fluorescence signal indicates that amplification products have been formed. At the start of the reaction the batches contained 600-fold (arrow 1), 6000-fold (arrow 2) and 60000-fold (arrow 3) diluted batches from example 3 with mixture 8 (M 1.1-T2C-300-3002 und M 2.1-T2C-300-3002). Arrow 4 shows the course of a negative control, here H₂O was used in the PCR to generate fragments instead of template. No amplification products were generated in the negative control. The amplification batch was performed with primer mix 1 and respective controller oligonucleotide. The BST polymerase was used in 1 to 120-fold dilution of the stock solution.

FIG. 29 (B) shows the melting curves belonging to the batches. The Y-axis shows the derivative of the fluorescence signal and the X-axis the temperature. A peak is visible, indicating that a uniform amplification product has been formed. The peaks marked with 1-3 belong to the batches that were prepared at the start of reaction mixture 8 (M 1.1-T2C-300-3002 und M 2.1-T2C-300-3002) in 600-fold, 6000- and 60000-fold dilutions. The curves marked with 4 comprise no product peak and belong to the negative control.

FIG. 30 shows the influence of the amount of polymerase used on the course of the reaction.

FIG. 30 (A) shows a typical course of the EvaGreen fluorescence signal during the amplification reaction. The change in the fluorescence signal of the EvaGreen dye is plotted on the Y axis and the reaction time (as cycle number) is shown on the X axis. The increase of the fluorescence signal indicates that amplification products have been formed. At the start of the reaction the batches contained 600-fold (arrow 1) diluted batches from example 3 with mixture 8 (M 1.1-T2C-300-3002 und M 2.1-T2C-300-3002). Arrow 2 shows the course of a negative control, here H₂O was used in the PCR to generate fragments instead of template. No amplification products were generated in the negative control. The amplification batch was performed with primer mix 2 and respective controller oligonucleotides. The BST polymerase was used in a 1:1200 dilution of the stock solution.

FIG. 30 (B) shows a typical course of the EvaGreen fluorescence signal during the amplification reaction. The change in the fluorescence signal of the EvaGreen dye is plotted on the Y axis and the reaction time (as cycle number) is shown on the X axis. The increase of the fluorescence signal indicates that amplification products have been formed. At the start of the reaction the batches contained 600-fold (arrow 1) diluted batches from example 3 with mixture 8 (M 1.1-T2C-300-3002 und M 2.1-T2C-300-3002). Arrow 2 shows the course of a negative control, here H₂O was used in the PCR to generate fragments instead of template. No amplification products were generated in the negative control. The amplification batch was performed with primer mix 2 and respective controller oligonucleotides. The BST polymerase was used in a 1:120 dilution of the stock solution.

1. A method for amplifying a nucleic acid chain, wherein
a sample comprising a start nucleic acid chain (M2.1) which in 5′-3′ orientation comprises the sequence segments M2.1.6, M2.1.5, M2.1.4, M2.1.3, M2.1.2, M2. 1.1 in immediate succession, wherein M2.1.6 comprises modified nucleotide building blocks so that M2.1.6 cannot serve as a template for the activity of a template-dependent nucleic acid polymerase,
is brought into contact with the following components:
a. a first template-dependent nucleic acid polymerase, particularly a DNA polymerase, as well as substrates of the template-dependent nucleic acid polymerase (particularly ribonucleoside triphosphates or deoxyribonucleoside triphosphates) and suitable cofactors,
b. two oligonucleotide primers P1.1 and P2.1, wherein in 5′-3′ orientation
i. P1.1 comprises two sequence segments P1.1.2 and P1.1.1 and
ii. P2.1 comprises two sequence segments P2.1.2 and P2.1.1
in immediate succession, wherein P1.1.1 comprises the sequence complementary [hybridizing] to M2.1.1 [can bind essentially sequence-specifically] as well as P2.1.1 comprises the sequence identical to M2.1.5 [binds to the extension product of P1.1 and therefrom initiates the synthesis of a sequence identical to M2.1] and P1.1.2 cannot bind to M2.1, and P2.1.2 cannot bind to the extension product of P1.1
and wherein
P1.1 and P2.1 in segment P1.1.2 and P2.1.2 comprise modified nucleotide building blocks such that P1.1.2 and P2.1.2 cannot serve as templates for the activity of the first template-dependent nucleic acid polymerase;
c. two controller oligonucleotides C1.1 and C2.1, wherein in 5′-3′ orientation
i. C1.1 comprises the sequence segments C1.1.3, C1.1.2 and C1.1.1 in immediate succession, wherein C1.1.3 is identical to M2.1.2, C1.1.2 is complementary to P1.1.1, and C1.1.1 can bind to P1.1.2
ii. C2.1 comprises the sequence segments C2.1.3, C2.1.2 and C2.1.1 in immediate succession, wherein C2.1.3 is complementary to M2.1.4, C2.1.2 is complementary to P2.1.1, and C2.1.1 can bind to P2.1.2
and wherein C1.1 and C2.1 comprise nucleotide building blocks modified in C1.1.2, C1.1.3 and C2.1.2, C2.1.3 such that C1.1 and C2.1 cannot serve as templates for the activity of the first template-dependent nucleic acid polymerase.

2. The method according to claim 1, wherein the template dependent nucleic acid polymerase synthesizes
an extension product P1.1-Ext starting from P1.1 and
an extension product P2.1-Ext starting from P2.1
and P1.1-Ext can form an extension product double strand with P2.1-Ext and wherein
the length of C1.1.3, C1.1.2, C1.1.3, C2.1.3, C2.1.2, C2.1.1 and optionally M2.1.3, and/or
the reaction conditions
are selected such that the extension product double strand is stable in the absence of C1.1 and C2.1 and is separated from each other in the presence of C1.1 and C2.1.

3. The method according to claim 1, characterized in that the modified nucleotide building blocks comprise 2′-O-alkylribonucleoside building blocks, particularly 2′-O-methylribonucleoside building blocks.

4. The method according to claim 1, characterized in that the sequence segments M2.1.5, M2.1.4, M2.1.3, M2.1.2 and M2.1.1 together have a length of 20 nucleotides to 200 nucleotides.

5. The method according to claim 1, characterized in that P1.1 and P2.1 each have a length in the range from 15 nucleotides to 100 nucleotides.

6. The method according to claim 1, characterized in that P1.1.2 and P2.1.2 each have a length in the range from 5 nucleotides to 85 nucleotides, particularly that P1.1.2 and P2.1.2 have between 50% and 300% of the length of the sequence segment P1.1.1 and P2.1.1 respectively.

7. The method according to claim 1, characterized in that C1.1 and C2.1 each have a length in the range of 20 nucleotides to 100 nucleotides.

8. The method according to claim 1, characterized in that [the region not covered by C1.1 or C2.1] M2.1.3 has a length of 0 to 50 nucleotides, particularly 0 to 25 nucleotides, particularly 20 nucleotides.

9. The method according to claim 1, characterized in that M2.1.3 comprises a length of 0 to 60 nucleotides, particularly 5 to 40 nucleotides, particularly 5 to 30 nucleotides.

10. The method according to claim 1, characterized in that the method is carried out essentially isothermally.

11. The method according to claim 10, characterized in that the reaction temperature is in the range of 50° C. to 70° C.

12. The method according to claim 1, wherein the start nucleic acid chain, M2.1, was obtained by the reaction of a second template-dependent nucleic acid polymerase via hybridizing an oligonucleotide primer containing the sequence P2.1.1 at its 3′ end with a target sequence TS4.1

13. The method according to claim 12, wherein the oligonucleotide primer containing the sequence P2.1.1 at its 3′ end is identical to P2.1.

14. The method according to claim 12, wherein the oligonucleotide primer containing the sequence P2.1.1 at its 3′ end consists only of the sequence P2.1.1.

15. A kit for carrying out a method according to claim 1, comprising:
a. two oligonucleotide primers P1.1 and P2.1, wherein in 5′-3′ orientation
i. P1.1 comprises two sequence segments P1.1.2 and P1.1.1 and
ii. P2.1 comprises two sequence segments P2.1.2 and P2.1.1
in immediate succession, wherein P1.1.1 comprises the sequence complementary [hybridizing] to a sequence segment M2.1.1 comprised in a target sequence [can bind essentially sequence-specific] as well as P2.1.1 comprises an identical sequence to the sequence segment M2.1.5 comprised in a target sequence [binds to the extension product of P1.1 and therefrom initiates the synthesis of a sequence identical to M2.1] and P1.1.2 cannot bind to M2.1, and P2.1.2 cannot bind to the extension product of P1
hybridizing a first oligonucleotide primer (P1.1) to the 3′-terminal region of a nucleic acid chain to be amplified, wherein the first oligonucleotide primer comprises the following regions:
a first region (P1.1.1) which can bind sequence-specifically to the 3′ terminal region (M2.1.1 and/or P2.1E1) of the nucleic acid chain to be amplified,
a second region (P1.1.2) (primer overhang) which adjoins the 5′ end of the first region or is connected thereto via a linker, wherein the second region can be bound by a first controller oligonucleotide (C1.1) and remains essentially uncopied by a polymerase used for the amplification under the selected reaction conditions;
of the complementary strand of the nucleic acid chain to be amplified, wherein the second oligonucleotide primer comprises the following regions:
a first region (P2.1.1) which can bind sequence-specifically to the 3′-terminal region of the complementary strand (P1.1E1)
a second region (P2.1.2) (overhang) which adjoins the 5′ end of the first region or is connected thereto via a linker, wherein the second region can be bound by a second controller oligonucleotide (C2.1) and remains essentially uncopied by a polymerase used for the amplification under the selected reaction conditions;
Extension of the first oligonucleotide primer (P1.1) to obtain a first primer extension product (P1.1-Ext) which comprises, in addition to the first oligonucleotide primer, a region synthesized by the polymerase;
Extension of the second oligonucleotide primer (P2.1) to obtain a second primer extension product (P2.1-Ext) which comprises, in addition to the second oligonucleotide primer, a synthesized region;
binding of a first controller oligonucleotide (C1.1) to the first primer extension product (P1.1-Ext), wherein the first controller oligonucleotide comprises the following regions:
a first region (C1.1.1) which can bind to the second region (overhang) of the first primer extension product (P1.1E6),
a second region (C1.1.2) which is essentially complementary to the first region of the second oligonucleotide primer (P1.1.1), and
a third region (C1.1.3) which is essentially complementary to at least part of the synthesized region of the first primer extension product (P1.1E4);
wherein the first controller oligonucleotide does not serve as a template for primer extension of the first oligonucleotide primer, and the first controller oligonucleotide binds to the first and second region of the first primer extension product to displace complementary segments of the other second strand (P2.1E1 and P2.1E2) of the nucleic acid chain to be amplified;
Binding a second controller oligonucleotide (C2.1) to the second primer extension product (P2.1-Ext), wherein the second controller oligonucleotide comprises the following regions
a first region (C2.1.1) which can bind to the second region of the second primer extension product (P2.1E6),
a second region (C2.1.2) which is essentially complementary to the first segment of the second oligonucleotide primer (P2.1.1), and
a third region (C2.1.3) which is essentially complementary to at least a part of the synthesized region of the second primer extension product (P2.1E4);
wherein the second controller oligonucleotide does not serve as a template for a primer extension of the second oligonucleotide primer, and the second controller oligonucleotide binds to the primer extension product to displace complementary segments of the first strand (P1.1E1 and P1.1E2) of the nucleic acid chain to be amplified, and wherein
P1.1 and P2.1 in segments P1.1.2 and P2.1.2 comprise modified nucleotide building blocks, so that P1.1.2 and P2.1.2 cannot serve as templates for the activity of the first template-dependent nucleic acid polymerase;
b. two controller oligonucleotides C1.1 and C2.1, wherein in 5′-3′
i. C1.1 comprises the sequence segments C1.1.3, C1.1.2 and C1.1.1 in immediate succession, wherein C1.1.3 is identical to M2.1.2, C1.1.2 is complementary to P1.1.1, and C1.1.1 can bind to P1.1.2,
ii. C2.1 comprises the sequence segments C2.1.3, C2.1.2 and C2.1.1 in immediate succession, wherein C2.1.3 is complementary to M2.1.4, C2.1.2 is complementary to P2.1.1, and C2.1.1 can bind to P2.1.2,
and wherein C1.1 and C2.1 comprise nucleotide building blocks modified in C1.1.2, C1.1.3 and C2.1.2, C2.1.3 such that C1.1 and C2.1 cannot serve as templates for the activity of the first template-dependent nucleic acid polymerase.

16. The kit according to claim 15, further comprising a first template-dependent nucleic acid polymerase, particularly a DNA polymerase, as well as optionally substrates of the DNA polymerase (particularly ribonucleoside triphosphates or deoxyribonucleoside triphosphates) and suitable cofactors, particularly a mesophilic template-dependent polymerase, particularly a mesophilic template-dependent polymerase without 5′-3′ exonuclease activity.

17. The kit according to claim 15, further comprising a second template-dependent nucleic acid polymerase, particularly a DNA polymerase, as well as optionally substrates of the template-dependent nucleic acid polymerase (particularly ribonucleoside triphosphates or deoxyribonucleoside triphosphates) and suitable cofactors, particularly a thermophilic template-dependent polymerase, particularly a thermophilic template-dependent polymerase with 5′-3′ exonuclease activity.


SEQUENCE LISTING (38 sequences)

SEQ ID NO: 1 - DNA, 54 bp, Artificial Sequence
  Primer
  misc_feature (31)..(42): 2′-O-Methyl Modification
  misc_feature (42)..(43): C3-linker
cacaatcatcaatactttttttacctgtatcucugaugcuuctacctgtattcc

SEQ ID NO: 2 - DNA, 54 bp, Artificial Sequence
  Primer
  misc_feature (31)..(42): 2′-O-Methyl Modification
  misc_feature (42)..(43): C3-linker
cacaatcatcaatacttttttgtcaaccatucucguagucucgtcaaccataat

SEQ ID NO: 3 - DNA, 74 bp, Homo sapiens
aagtcaaccataatggactacttcgagcagatccattagaggtctggacaggcaaggaatacaggtattttgaa

SEQ ID NO: 4 - DNA, 84 bp, Homo sapiens
aagtcaaccataatggactacttcgagcagatccattagctcctacgccaggtctggacaggcaaggaatacaggtattttgaa

SEQ ID NO: 5 - DNA, 94 bp, Homo sapiens
aagtcaaccataatggactacttcgagcagatccattagctcctctggatatagacgccaggtctggacaggcaaggaatacaggtattttgaa

SEQ ID NO: 6 - DNA, 134 bp, Homo sapiens
aagtcaaccataatatcatgagagacatcgcctctgggctaataattagctcctctggatatagacgccaggtggactacttctaatctgtaagagcagatccctggacaggcaaggaatacaggtattttgaa

SEQ ID NO: 7 - DNA, 124 bp, Homo sapiens
aagtcaaccataatatcatgagagacatcgcctctgggctaataattagctcctacgccaggtggactacttctaatctgtaagagcagatccctggacaggcaaggaatacaggtattttgaa

SEQ ID NO: 8 - DNA, 105 bp, Homo sapiens
aagtcaaccataatatcatgagagacatcgcctctgggctaataggactacttctaatctgtaagagcagatccctggacaggcaaggaatacaggtattttgaa

SEQ ID NO: 9 - DNA, 114 bp, Artificial Sequence
  Extension product
  misc_feature (31)..(42): 2′-O-Methyl Modification
  misc_feature (42)..(43): C3-linker
cacaatcatcaatacttttttgtcaaccatucucguagucucgtcaaccataatggactacttcgagcagatccattagaggtctggacaggcaaggaatacaggtattttgaa

SEQ ID NO: 10 - DNA, 109 bp, Artificial Sequence
  Extension product
  misc_feature (31)..(42): 2′-O-Methyl Modification
  misc_feature (42)..(43): C3-linker
cacaatcatcaatactttttttacctgtatcucugaugcuuctacctgtattccttgcctgtccagacctctaatggatctgctcgaagtagtccattatggttgactt

SEQ ID NO: 11 - DNA, 124 bp, Artificial Sequence
  Extension product
  misc_feature (31)..(42): 2′-O-Methyl Modification
  misc_feature (42)..(43): C3-linker
cacaatcatcaatacttttttgtcaaccatucucguagucucgtcaaccataatggactacttcgagcagatccattagctcctacgccaggtctggacaggcaaggaatacaggtattttgaa

SEQ ID NO: 12 - DNA, 119 bp, Artificial Sequence
  Extension product
  misc_feature (31)..(42): 2′-O-Methyl Modification
  misc_feature (42)..(43): C3-linker
cacaatcatcaatactttttttacctgtatcucugaugcuuctacctgtattccttgcctgtccagacctggcgtaggagctaatggatctgctcgaagtagtccattatggttgactt

SEQ ID NO: 13 - DNA, 134 bp, Artificial Sequence
  Extension product
  misc_feature (31)..(42): 2′-O-Methyl Modification
  misc_feature (42)..(43): C3-linker
cacaatcatcaatacttttttgtcaaccatucucguagucucgtcaaccataatggactacttcgagcagatccattagctcctctggatatagacgccaggtctggacaggcaaggaatacaggtattttgaa

SEQ ID NO: 14 - DNA, 129 bp, Artificial Sequence
  Extension product
  misc_feature (31)..(42): 2′-O-Methyl Modification
  misc_feature (42)..(43): C3-linker
cacaatcatcaatactttttttacctgtatcucugaugcuuctacctgtattccttgcctgtccagacctggcgtctatatccagaggagctaatggatctgctcgaagtagtccattatggttgactt

SEQ ID NO: 15 - DNA, 174 bp, Artificial Sequence
  Extension product
  misc_feature (31)..(42): 2′-O-Methyl Modification
  misc_feature (42)..(43): C3-linker
cacaatcatcaatacttttttgtcaaccatucucguagucucgtcaaccataatatcatgagagacatcgcctctgggctaataattagctcctctggatatagacgccaggtggactacttctaatctgtaagagcagatccctggacaggcaaggaatacaggtattttgaa

SEQ ID NO: 16 - DNA, 169 bp, Artificial Sequence
  Extension product
  misc_feature (31)..(42): 2′-O-Methyl Modification
  misc_feature (42)..(43): C3-linker
cacaatcatcaatactttttttacctgtatcucugaugcuuctacctgtattccttgcctgtccagggatctgctcttacagattagaagtagtccacctggcgtctatatccagaggagctaattattagcccagaggcgatgtctctcatgatattatggttgactt

SEQ ID NO: 17 - DNA, 164 bp, Artificial Sequence
  Extension product
  misc_feature (31)..(42): 2′-O-Methyl Modification
  misc_feature (42)..(43): C3-linker
cacaatcatcaatacttttttgtcaaccatucucguagucucgtcaaccataatatcatgagagacatcgcctctgggctaataattagctcctacgccaggtggactacttctaatctgtaagagcagatccctggacaggcaaggaatacaggtattttgaa

SEQ ID NO: 18 - DNA, 159 bp, Artificial Sequence
  Extension product
  misc_feature (31)..(42): 2′-O-Methyl Modification
  misc_feature (42)..(43): C3-linker
cacaatcatcaatactttttttacctgtatcucugaugcuuctacctgtattccttgcctgtccagggatctgctcttacagattagaagtagtccacctggcgtaggagctaattattagcccagaggcgatgtctctcatgatattatggttgactt

SEQ ID NO: 19 - DNA, 145 bp, Artificial Sequence
  Extension product
  misc_feature (31)..(42): 2′-O-Methyl Modification
  misc_feature (42)..(43): C3-linker
cacaatcatcaatacttttttgtcaaccatucucguagucucgtcaaccataatatcatgagagacatcgcctctgggctaataggactacttctaatctgtaagagcagatccctggacaggcaaggaatacaggtattttgaa

SEQ ID NO: 20 - DNA, 140 bp, Artificial Sequence
  Extension product
  misc_feature (31)..(42): 2′-O-Methyl Modification
  misc_feature (42)..(43): C3-linker
cacaatcatcaatactttttttacctgtatcucugaugcuuctacctgtattccttgcctgtccagggatctgctcttacagattagaagtagtcctattagcccagaggcgatgtctctcatgatattatggttgactt

SEQ ID NO: 21 - DNA, 24 bp, Artificial Sequence
  Block oligonucleotide
  misc_feature (1)..(18): 2′-O-Methyl Modification
  misc_feature (18)..(19): C3-linker
  misc_feature (24)..(24): 34-phosphate group
cucugaugcuucuaccugtattaa

SEQ ID NO: 22 - DNA, 28 bp, Artificial Sequence
  Block oligonucleotide
  misc_feature (1)..(17): 2′-O-Methyl Modification
  misc_feature (17)..(18): HEG-linker
  misc_feature (28)..(28): 34-phosphate group
ucucguagucucgucaaccataatgaaa

SEQ ID NO: 23 - DNA, 37 bp, Artificial Sequence
  Controller oligonucleotide
  misc_feature (1)..(22): 2′-O-Methyl Modification
  misc_feature (37)..(37): 34- phosphate group
acuggacaggcaaggaauacaggtagaagcatcagag

SEQ ID NO: 24 - DNA, 47 bp, Artificial Sequence
  Controller oligonucleotide
  misc_feature (1)..(28): 2′-O-Methyl Modification
  misc_feature (47)..(47): 34-phosphate group
aggaucugcucgaaguaguccauuauggttgacgagactacgagaag

SEQ ID NO: 25 - DNA, 67 bp, Artificial Sequence
  Controller oligonucleotide
  misc_feature (1)..(52): 2′-O-Methyl Modification
  misc_feature (67)..(67): 34-phosphate group
aggacuacuucuaaucuguaagagcagaucccuggacaggcaaggaauacaggtagaagcatcagag

SEQ ID NO: 26 - DNA, 57 bp, Artificial Sequence
  Controller oligonucleotide
  misc_feature (1)..(38): 2′-O-Methyl Modification
  misc_feature (57)..(57): 34-phosphate group
auauuagcccagaggcgaugucucucaugauauuauggttgacgagactacgagaag

SEQ ID NO: 27 - DNA, 91 bp, Homo sapiens
tacctgtattccttgcctgtccagggatctgctcttacagattaaaatattagcccagaggcgatgtctctcatgatacaggtattttgac

SEQ ID NO: 28 - DNA, 102 bp, Homo sapiens
tacctgtattccttgcctgtccagggatctgctcttacagattacttagagcttagagtattagcccagaggcgatgtctctcatgatacaggtattttgac

SEQ ID NO: 29 - DNA, 56 bp, Artificial Sequence
  Primer
  misc_feature (31)..(42): 2′-O-Methyl Modification
  misc_feature (42)..(43): C3-linker
cacaatcatcaatacttttttgtcaaaatacucugaugcucugtcaaaatacctgt

SEQ ID NO: 30 - DNA, 28 bp, Artificial Sequence
  Primer
  misc_feature (1)..(12): 2′-O-Methyl Modification
  misc_feature (12)..(13): C3-linker
cucugaugcuuctacctgtattccttgc

SEQ ID NO: 31 - DNA, 133 bp, Artificial Sequence
  Extension product
  misc_feature (31)..(42): 2′-O-Methyl Modification
  misc_feature (42)..(43): C3-linker
cacaatcatcaatacttttttgtcaaaatacucugaugcucugtcaaaatacctgtatcatgagagacatcgcctctgggctaatattttaatctgtaagagcagatccctggacaggcaaggaatacaggta

SEQ ID NO: 32 - DNA, 103 bp, Artificial Sequence
  Extension product
  misc_feature (1)..(12): 2′-O-Methyl Modification
  misc_feature (12)..(13): C3-linker
cucugaugcuuctacctgtattccttgcctgtccagggatctgctcttacagattaaaatattagcccagaggcgatgtctctcatgatacaggtattttgac

SEQ ID NO: 33 - DNA, 144 bp, Artificial Sequence
  Extension product
  misc_feature (31)..(42): 2′-O-Methyl Modification
  misc_feature (42)..(43): C3-linker
cacaatcatcaatacttttttgtcaaaatacucugaugcucugtcaaaatacctgtatcatgagagacatcgcctctgggctaatactctaagctctaagtaatctgtaagagcagatccctggacaggcaaggaatacaggta

SEQ ID NO: 34 - DNA, 114 bp, Artificial Sequence
  Extension product
  misc_feature (1)..(12): 2′-O-Methyl Modification
  misc_feature (12)..(13): C3-linker
cucugaugcuuctacctgtattccttgcctgtccagggatctgctcttacagattacttagagcttagagtattagcccagaggcgatgtctctcatgatacaggtattttgac

SEQ ID NO: 35 - DNA, 24 bp, Artificial Sequence
  Primer
  misc_feature (1)..(1): 5′-TMR modification
  misc_feature (1)..(12): 2′-O-Methyl Modification
  misc_feature (12)..(13): C3-linker
cucugaugcucugtcaaaatacct

SEQ ID NO: 36 - DNA, 24 bp, Artificial Sequence
  Primer
  misc_feature (1)..(12): 2′-O-Methyl Modification
  misc_feature (12)..(13): C3-linker
cucugaugcuuctacctgtattcc

SEQ ID NO: 37 - DNA, 54 bp, Artificial Sequence
  Primer
  misc_feature (31)..(42): 2′-O-Methyl Modification
  misc_feature (42)..(43): C3-linker
cacaatcatcaatacttttttgtcaaaatacucugaugcucugtcaaaatacct

SEQ ID NO: 38 - DNA, 58 bp, Artificial Sequence
  Controller oligonucleotide
  misc_feature (1)..(39): 2′-O-Methyl Modification
  misc_feature (58)..(58): 34-phosphate group
uauuagcccagaggcgaugucucucaugauacagguauuttgacagagcatcagagag